(19) United States
(12) Patent Application Publication
(10) Pub. No.: US 2004/0010207 A1
(43) Pub. Date: Jan. 15, 2004

(54) Self-contained, automatic transcutaneous physiologic sensing system

(76) Inventors: J. Christopher Flaherty, Topsfield, MA (US); William Gorman, South Hamilton, MA (US); Duane R. Mason, East Dennis, MA (US)

Correspondence Address: McDermott, Will & Emery, 28 State Street, Boston, MA, 02109, US

(21) Appl. No.: 10/195,745

(22) Filed: Jul. 15, 2002

(51) Int. Cl. A61B 5/00

(52) U.S. Cl. 600/573

(57) ABSTRACT

A device for monitoring a physiological parameter of a person includes a sensor device for measuring a physiological parameter associated with the person; a processor for processing measurements of the physiological parameter generated by the sensor device; a transcutaneous member coupled between the sensor device and the processor, including a penetrating member at a distal end thereof for piercing the skin of the person; a housing containing the sensor device, the transcutaneous member and the processor, the housing including an exit port for receiving the distal end of the transcutaneous member upon injection of the distal end into the person and means for securing a first wall of the housing to the skin of the person; and an injection activation device including a driving mechanism contacting the transcutaneous member for driving the penetrating member from a first position within the housing, through the exit port to a second position, external to the housing and into the skin of the person.

### FIELD OF THE INVENTION

The present invention relates generally to devices for sensing physiological parameters of a patient and more particularly to small, disposable, portable patient-worn devices with automatic transcutaneous injection devices that can be used to sense physiological parameters and optionally transcutaneously deliver fluid drugs and non-fluidic therapeutic devices safely and simply to a patient. Even more particularly, the present invention relates a transcutaneous assembly that allows placement of a transcutaneous member including a sensor assembly safely and automatically, and does not require the disposal of a sharp, contaminated needle.

### BACKGROUND OF THE INVENTION

There are many physiologic conditions that require constant or periodic monitoring to insure that a person who is suffering from a particular condition receives the proper attention if potentially damaging physiological changes occur in the person. Oftentimes, in response to a physiologic change, a person is required to administer a medicine to compensate for the change that has occurred.

For example, a person who is diabetic must monitor his or her blood glucose levels to insure that the blood glucose does not drop to a level that may cause harm to the person. In such a case, the person would monitor his or her blood glucose levels by drawing a small amount of blood and testing the blood, typically with the use of an electronic blood glucose sensing device. Based on the results of the test, the person can then inject an amount of insulin to bring the blood glucose level back to the “normal” level. While such a testing system enables a person to monitor his or her glucose level, it requires that the person remember to perform the required test at the required time intervals, requires that the person interpret the results correctly and also exposes the person to the possibility of infection resulting from the puncture wound resulting from the blood withdrawal.

Accordingly, there is a need for a programmable sensing system that is precise and reliable and can offer a simple to use alternative to manually monitoring physiologic conditions of a person.

### SUMMARY OF THE INVENTION

The applicant has determined that a sophisticated ambulatory sensor device that can be programmed to reliably sense certain physiological parameters of fluid withdrawn from the person or which is sampled from within the person, yet is small, lightweight and low cost, is needed. The sensing devices of the present invention are simple in design, and inexpensive and easy to manufacture, to further reduce the size, complexity and costs of the devices, such that the devices or portions thereof lend themselves to being small and disposable in nature. In addition, the sensing devices may include a transcutaneous infusion assembly that allows placement of a transcutaneous member safely and automatically, and does not require the disposal of a sharp, contaminated needle.

An inexpensive device allows greater flexibility in prescribing the device for use by reducing the financial burden on healthcare insurance providers, hospitals and patient care centers as well as patients themselves. In addition, low cost devices make it more practical for a patient to have one or more replacement devices readily available. If the primary device is lost or becomes dysfunctional, availability of the replacement eliminates costly expedited repair and avoids periods of discontinued ambulatory therapy.

According to one embodiment of the invention, a device for monitoring a physiological parameter of a person includes a sensor device for measuring a physiological parameter associated with the person, a processor for processing measurements of the physiological parameter generated by the sensor device, a transcutaneous member coupled to the sensor device and the processor, including a penetrating member at a distal end thereof for piercing the skin of the person, a housing containing the sensor device, the transcutaneous member and the processor, the housing including an exit port for receiving the distal end of the transcutaneous member upon injection of the distal end into the person and means for securing a first wall of the housing to the skin of the person and an injection activation device including a driving mechanism contacting the transcutaneous member for driving the penetrating member from a first position within the housing, through the exit port to a second position, external to the housing and into the skin of the person.

At least a sample receiving portion of the sensor device may be disposed at the distal end of the transcutaneous member. The physiological parameter may be at least one of blood glucose level, blood gas level, body temperature, exposure to an external agent, allergic reactions, respiration, arrhythmia, blood cell count, blood flow rate, average blood clotting time, thrombogenicity, blood oxygen content, blood pH and toxicity levels. The driving mechanism of the injection activation device may include a plunger having a body portion extending through an aperture in a second wall of the housing and in frictional contact with the distal end of the fluid transport device, such that the application of a longitudinal force to the plunger drives the penetrating member from the first position to the second position. The plunger may include a friction member disposed on the body portion, the friction member causing the body portion of the plunger to have a width dimension which is slightly larger than a width dimension of the aperture of the housing, thus requiring a specific longitudinal force to be applied to the plunger to enable the friction member to pass through the aperture, the specific force being translated to the distal end of the fluid transport device. The friction member may be an annular flange. The plunger may further include a head portion for stopping travel of the plunger by contacting the housing. The plunger may be removable from the housing after the penetrating member is driven to the second position. The driving mechanism of the injection activation device ma include a plunger contained within the housing, the plunger having a first end including a lateral protrusion and a second end in frictional contact with the distal end of the transcutaneous member, the injection activation device further including a biasing spring for biasing the plunger for driving the penetrating member from the first position to the second position, and the lateral protrusion being in contact with an internal ridge of the housing, with the penetrating member in the first position, thereby preventing the plunger from driving the penetrating member from the first position to the second position. The housing may include an actuator for urging the lateral protrusion from the internal ridge, thereby causing the plunger to drive the penetrating member from the first position to the second position. The actuator may include a finger coupled to an inside surface of a flexible wall portion of the housing, a distal end of the finger being in contact with the lateral protrusion such that an application of pressure to the flexible wall portion causes the finger to urge the lateral protrusion from the ridge, thereby causing the plunger to drive the penetrating member from the first position to the second position. The distal end of the finger, upon the application of pressure to the flexible wall portion, may move in same the direction as the flexible wall portion. The distal end of the finger, upon the application of pressure to the flexible wall portion, moves in a substantially opposite direction as the flexible wall portion. The finger may include a pivot which causes the distal end of the finger to move in a direction substantially opposite that of the flexible wall portion. The driving mechanism of the injection activation device comprises a pivoting arm and the injection activation device further includes a latch assembly, the pivoting arm having a proximal end pivotally coupled to an inside surface of a wall of the housing and a distal end in contact with the latch assembly integral with a side wall of the housing, the fluid transport device being coupled to the arm such that when the distal end of the arm is in contact with the latch assembly, the penetrating member is in the first position. The injection activation device may further include a biasing spring attached between the proximal and distal ends of the arm and a wall of the housing, the biasing spring urging the arm to drive the penetrating member to the second position. The latch assembly includes a latch for contacting the distal end of the pivoting arm to prevent the pivoting arm from driving the penetrating member from the first position to the second position under the influence of the biasing spring and a latch release mechanism for moving the latch out of contact with the distal end of the pivoting arm, thereby enabling the pivoting arm to drive the penetrating member from the first position to the second position under the influence of the biasing spring. The device of latch release mechanism may include an electrically driven actuator coupled between the latch and the side wall of the housing, such that, upon the application of a charge to the electrically driven actuator, the electrically driven actuator activates to pull the latch out of contact with the distal end of the pivoting arm. The electrically driven actuator may include one of a shape memory alloy, a shape memory polymer, a piezo electric actuator and a solenoid. The device may further include a local processor connected to the latch release mechanism and programmed to apply a charge to the electrically driven actuator based on injection instructions and a wireless receiver connected to the local processor for receiving injection instructions from a separate, remote control device and delivering the injection instructions to the local processor. The housing may be free of user input components for providing injection instructions to the local processor. The device may further include a remote control device separate from the transcutaneous member which includes a remote processor, user interface components connected to the remote processor for transmitting the injection instructions to the remote processor and a transmitter connected to the remote processor for transmitting the injection instructions to the receiver of the device for monitoring a physiological parameter. The latch release mechanism includes a mechanical lever coupled to the latch and protruding through the side wall, such that, upon the lever being pulled away from the housing, the latch is pulled out of contact with the distal end of the pivoting arm. The injection activation device may include a discrete secondary housing, the plunger including a first end having a lateral protrusion and a second end in frictional contact with the distal end of the fluid transport device, the second end of the plunger extending from within the secondary housing, out of a distal end thereof into the aperture of the housing and into frictional contact with the distal end of the fluid transport device. The injection activation device may further include a biasing spring coupled between the first end of the plunger and a proximal end of the secondary housing within the secondary housing for biasing the plunger for driving the penetrating member from the first position to the second position, the lateral protrusion being in contact with an internal ridge of the secondary housing, with the penetrating member in the first position, thereby preventing the plunger from driving the penetrating member from the first position to the second position. The secondary housing may include an actuator for urging the lateral protrusion from the internal ridge, thereby causing the plunger to drive the penetrating member from the first position to the second position. The injection activation device may include a discrete secondary housing, the plunger including a first end having a lateral protrusion and a second end in frictional contact with the distal end of the fluid transport device, the second end of the plunger extending from within the secondary housing, out of a distal end thereof into the aperture of the housing and into frictional contact with the distal end of the fluid transport device. The injection activation device may further include a biasing spring coupled between the first end of the plunger and a proximal end of the secondary housing within the secondary housing for biasing the plunger for driving the penetrating member from the first position to the second position, the lateral protrusion being in contact with a latch assembly of the secondary housing, with the penetrating member in the first position, thereby preventing the plunger from driving the penetrating member from the first position to the second position. The latch assembly may include a latch for contacting the lateral protrusion of the plunger to prevent the plunger from driving the penetrating member from the first position to the second position under the influence of the biasing spring and a latch release mechanism coupled to the housing for moving the latch out of contact with the lateral protrusion, thereby enabling the plunger to drive the penetrating member from the first position to the second position under the influence of the biasing spring. The latch release mechanism may include an electrically driven actuator coupled between the latch and the side wall of the housing, such that, upon the application of a charge to the electrically driven actuator, the electrically driven actuator activates to pull the latch out of contact with the distal end of the pivoting arm. The electrically driven actuator may include one of a shape memory alloy, a shape memory polymer, a piezo electric actuator and a solenoid. The device may further include a local processor housed in the secondary housing, the local processor being connected to the latch release mechanism and programmed to apply a charge to the electrically driven actuator based on injection instructions; and a wireless receiver connected to the local processor for receiving injection instructions from a separate, remote control device and delivering the injection instructions to the local processor. The latch release mechanism may include a mechanical lever coupled to the latch and protruding through the side wall, such that, upon an application of force to the lever, the latch is moved out of contact with the distal end of the pivoting arm. The driving mechanism may include a plunger having a first end in frictional contact with the distal end of the fluid transport device, the plunger being biased to drive the penetrating member from the first position to the second position, the injection activation device further comprising a latch for contacting the plunger to maintain the penetrating member in the first position, the latch including an electrically driven actuator coupled to the latch, such that, upon the application of a charge to the electrically driven actuator, the electrically driven actuator activates to pull the latch out of contact with the plunger, thereby enabling the plunger to drive the penetrating means from the first position to the second position. The electrically driven actuator may include one of a shape memory alloy, a shape memory polymer, a piezo electric actuator and a solenoid. The device may further include a local processor connected to the latch release mechanism and programmed to apply a charge to the electrically driven actuator based on injection instructions; and a wireless receiver connected to the local processor for receiving injection instructions from a separate, remote control device and delivering the injection instructions to the local processor. The sensor device may include physiological parameter sensing means for performing a sampling operation on a sample received by the sample receiving portion to monitor a physiological parameter of the person. The physiological parameter may be at least one of blood glucose level, blood gas level, exposure to an external agent, allergic reactions, respiration, arrhythmia, blood cell count, blood flow rate, average blood clotting time, thrombogenicity, blood oxygen content, blood pH and toxicity levels. The device may further include a reservoir for containing a medicine to be delivered to the person and a fluid transport device, enclosed within the housing, for dispensing medicine from the reservoir to the person, the fluid transport device including a proximal end in fluid communication with the reservoir and a distal end having a penetrating member for piercing the skin of the person to facilitate the delivery of medicine to the person through the fluid transport device. The device may further include a second injection activation device including a second driving mechanism contacting the fluid transport device for driving the penetrating member from the first position within the housing, through the exit port to the second position, external to the housing and into the skin of the person. The sensor device includes means for instructing the second injection activation device to drive the fluid transport from the first position within the housing, through the exit port to the second position, external to the housing and into the skin of the person and to transport an amount of medicine to the person, based on the sampling operation. The processor may include an injection activation instruction generation portion for providing injection activation instructions to the injection activation device based on a trigger signal provided to the injection activation instruction generation portion. The trigger signal may be generated within the processor based on timing instructions programmed into the processor, the timing instructions causing the trigger signal to be provided to the injection activation instruction generation portion at predetermined time intervals. The trigger signal may be generated within the processor based on a sensor input to the processor from a second sensor which monitors at least one environmental parameter, the sensor input causing the trigger signal to be provided to the injection activation instruction generation portion upon the environmental parameter reaching a predetermined level. The second sensor may be disposed within the housing. The second sensor may be located externally from the housing. The second sensor may include a transmitter for transmitting the sensor input to a receiver associated with the processor. The environmental parameter may include at least one of temperature, pressure, oxygen level, light and the presence of a chemical agent.

According to another embodiment of the invention, a device for monitoring fluid from a person includes a sensor device for receiving fluid from the person, a fluid transport device for with drawing fluid from the person to the sensor device, the fluid transport device including a proximal end in fluid communication with the sensor device and a distal end having a penetrating member for piercing the skin of the person to facilitate the withdrawal of fluid to the person through the fluid transport device, a housing containing the sensor device and the fluid transport device, the housing including an exit port for receiving the distal end of the fluid transport device upon injection of the distal end into the person and means for securing a first wall of the housing to the skin of the person and an injection activation device including a driving mechanism contacting the fluid transport device for driving the penetrating member from a first position within the housing, through the exit port to a second position, external to the housing and into the skin of the person.

The driving mechanism of the injection activation device may include a plunger having a body portion extending through an aperture in a second wall of the housing and in frictional contact with the distal end of the fluid transport device, such that the application of a longitudinal force to the plunger drives the penetrating member from the first position to the second position. The plunger may include a friction member disposed on the body portion, the friction member causing the body portion of the plunger to have a width dimension which is slightly larger than a width dimension of the aperture of the housing, thus requiring a specific longitudinal force to be applied to the plunger to enable the friction member to pass through the aperture, the specific force being translated to the distal end of the fluid transport device. The friction member may be an annular flange. The plunger may further include a head portion for stopping travel of the plunger by contacting the housing. The plunger may be removable from the housing after the penetrating member is driven to the second position. The driving mechanism of the injection activation device may include a plunger contained within the housing, the plunger having a first end including a lateral protrusion and a second end in frictional contact with the distal end of the fluid transport device, the injection activation device further including a biasing spring for biasing the plunger for driving the penetrating member from the first position to the second position, and the lateral protrusion being in contact with an internal ridge of the housing, with the penetrating member in the first position, thereby preventing the plunger from driving the penetrating member from the first position to the second position. The housing may include an actuator for urging the lateral protrusion from the internal ridge, thereby causing the plunger to drive the penetrating member from the first position to the second position. The actuator may include a finger coupled to an inside surface of a flexible wall portion of the housing, a distal end of the finger being in contact with the lateral protrusion such that an application of pressure to the flexible wall portion causes the finger to urge the lateral protrusion from the ridge, thereby causing the plunger to drive the penetrating member from the first position to the second position. The distal end of the finger, upon the application of pressure to the flexible wall portion, may move in same the direction as the flexible wall portion. The distal end of the finger, upon the application of pressure to the flexible wall portion, may move in a substantially opposite direction as the flexible wall portion. The finger may include a pivot which causes the distal end of the finger to move in a direction substantially opposite that of the flexible wall portion. The driving mechanism of the injection activation device may include a pivoting arm and the injection activation device further includes a latch assembly, the pivoting arm having a proximal end pivotally coupled to an inside surface of a wall of the housing and a distal end in contact with the latch assembly integral with a side wall of the housing, the fluid transport device being coupled to the arm such that when the distal end of the arm is in contact with the latch assembly, the penetrating member is in the first position. The injection activation device may further include a biasing spring attached between the proximal and distal ends of the arm and a wall of the housing, the biasing spring urging the arm to drive the penetrating member to the second position; and the latch assembly may include a latch for contacting the distal end of the pivoting arm to prevent the pivoting arm from driving the penetrating member from the first position to the second position under the influence of the biasing spring and a latch release mechanism for moving the latch out of contact with the distal end of the pivoting arm, thereby enabling the pivoting arm to drive the penetrating member from the first position to the second position under the influence of the biasing spring. The latch release mechanism may include an electrically driven actuator coupled between the latch and the side wall of the housing, such that, upon the application of a charge to the electrically driven actuator, the electrically driven actuator activates to pull the latch out of contact with the distal end of the pivoting arm. The electrically driven actuator may include one of a shape memory alloy, a shape memory polymer, a piezo electric actuator and a solenoid. The device may further include a local processor connected to the latch release mechanism and programmed to apply a charge to the electrically driven actuator based on injection instructions and a wireless receiver connected to the local processor for receiving injection instructions from a separate, remote control device and delivering the injection instructions to the local processor. The housing may be free of user input components for providing injection instructions to the local processor. The device may further include a remote control device separate from the fluid delivery device having a remote processor, user interface components connected to the remote processor for transmitting the injection instructions to the remote processor and a transmitter connected to the remote processor for transmitting the injection instructions to the receiver of the fluid delivery device. The latch release mechanism includes a mechanical lever coupled to the latch and protruding through the side wall, such that, upon the lever being pulled away from the housing, the latch is pulled out of contact with the distal end of the pivoting arm. The injection activation device may include a discrete secondary housing, the plunger including a first end having a lateral protrusion and a second end in frictional contact with the distal end of the fluid transport device, the second end of the plunger extending from within the secondary housing, out of a distal end thereof into the aperture of the housing and into frictional contact with the distal end of the fluid transport device. The injection activation device may further include a biasing spring coupled between the first end of the plunger and a proximal end of the secondary housing within the secondary housing for biasing the plunger for driving the penetrating member from the first position to the second position, the lateral protrusion being in contact with an internal ridge of the secondary housing, with the penetrating member in the first position, thereby preventing the plunger from driving the penetrating member from the first position to the second position. The secondary housing may include an actuator for urging the lateral protrusion from the internal ridge, thereby causing the plunger to drive the penetrating member from the first position to the second position. The injection activation device includes a discrete secondary housing, the plunger including a first end having a lateral protrusion and a second end in frictional contact with the distal end of the fluid transport device, the second end of the plunger extending from within the secondary housing, out of a distal end thereof into the aperture of the housing and into frictional contact with the distal end of the fluid transport device. The injection activation device may further include a biasing spring coupled between the first end of the plunger and a proximal end of the secondary housing within the secondary housing for biasing the plunger for driving the penetrating member from the first position to the second position, the lateral protrusion being in contact with a latch assembly of the secondary housing, with the penetrating member in the first position, thereby preventing the plunger from driving the penetrating member from the first position to the second position. The latch assembly may include a latch for contacting the lateral protrusion of the plunger to prevent the plunger from driving the penetrating member from the first position to the second position under the influence of the biasing spring and a latch release mechanism coupled to the housing for moving the latch out of contact with the lateral protrusion, thereby enabling the plunger to drive the penetrating member from the first position to the second position under the influence of the biasing spring. The latch release mechanism may include an electrically driven actuator coupled between the latch and the side wall of the housing, such that, upon the application of a charge to the electrically driven actuator, the electrically driven actuator activates to pull the latch out of contact with the distal end of the pivoting arm. The driving mechanism may include a plunger having a first end in frictional contact with the distal end of the fluid transport device, the plunger being biased to drive the penetrating member from the first position to the second position, the injection activation device further comprising a latch for contacting the plunger to maintain the penetrating member in the first position, the latch including an electrically driven actuator coupled to the latch, such that, upon the application of a charge to the electrically driven actuator, the electrically driven actuator activates to pull the latch out of contact with the plunger, thereby enabling the plunger to drive the penetrating means from the first position to the second position. The device may further include a local processor connected to the latch release mechanism and programmed to apply a charge to the electrically driven actuator based on injection instructions; and a wireless receiver connected to the local processor for receiving injection instructions from a separate, remote control device and delivering the injection instructions to the local processor. The sensor device includes physiological parameter sensing means for performing a sampling operation on the fluid to monitor a physiological parameter of the person. The physiological parameter is at least one of blood glucose level, blood gas level exposure to an external agent, allergic reactions, respiration, arrhythmia, blood cell count, blood flow rate, average blood clotting time, thrombogenicity, blood oxygen content, blood pH and toxicity levels. The device may further include a reservoir for containing a medicine to be delivered to the person and a second fluid transport device, enclosed within the housing, for dispensing medicine from the reservoir to the person, the fluid transport device including a proximal end in fluid communication with the reservoir and a distal end having a penetrating member for piercing the skin of the person to facilitate the delivery of medicine to the person through the fluid transport device. The device may further include a second injection activation device including a second driving mechanism contacting the second fluid transport device for driving the penetrating member from the first position within the housing, through the exit port to the second position, external to the housing and into the skin of the person. The sensor device may include means for instructing the second injection activation device to drive the second fluid transport from the first position within the housing, through the exit port to the second position, external to the housing and into the skin of the person and to transport an amount of medicine to the person, based on the sampling operation.

According to yet another embodiment of the invention, a device for monitoring a physiological parameter of a person includes a sensor device for measuring a physiological parameter associated with the person, a processor for processing measurements of the physiological parameter-generated by the sensor device, a transcutaneous member coupled to the sensor device and the processor, including a proximal end, a penetrating member at a distal end thereof for piercing the skin of the person and a medial portion disposed between the proximal and distal ends, a housing containing the sensor device, the transcutaneous member and the processor, the housing including an exit port for receiving the distal end of the transcutaneous member upon injection of the penetrating member into the person and means for securing a first wall of the housing to the skin of the person and an injection activation device including a driving mechanism contacting the proximal end of the transcutaneous member for driving the penetrating member from a first position within the housing, through the exit port to a second position, external to the housing and into the skin of the person. The medial portion is disposed substantially parallel to the first wall of the housing, the transcutaneous member includes a retention device which, with the penetrating member in the first position, is biased against a latch assembly of the injection activation device by a biasing spring of the injection activation device, which is coupled between the retention device and an internal ridge of the housing, the biasing spring being in an energized state such that, upon activating the latch assembly, the biasing spring drives the transcutaneous member in a direction of travel substantially parallel to the first wall, resulting in the penetrating member being driven from the first position to the second position.

The distal end of the transcutaneous member may be flexible and the housing may include a deflecting device in the path of travel of the transcutaneous member. Upon activating the latch assembly, the distal end of the transcutaneous member may contact the deflecting device which causes the distal end of the transcutaneous member to be deflected from the direction of travel substantially parallel to the first wall of the housing to a second direction of travel at an angle of at least 15. The second direction of travel may be up to 90. The latch assembly may include a latch for contacting the retention device of the transcutaneous member to prevent the biasing spring from driving the penetrating member from the first position to the second position and a latch release mechanism coupled to the housing for moving the latch out of contact with the retention device, thereby enabling the biasing spring to drive the penetrating member from the first position to the second position. The latch release mechanism may include an electrically driven actuator coupled between the latch and the housing, such that, upon the application of a charge to the electrically driven actuator, the shape memory allow wire contracts, pulling the latch out of contact with the retention device of the transcutaneous member. The latch release mechanism may include a mechanical lever coupled to the latch and protruding through the side wall, such that, upon an application of force to the lever, the latch is moved out of contact with the retention device. The biasing spring may include one of a torsional spring, a coil spring, a helical spring, a compression spring, an extension spring, an air spring, a wave spring, a conical spring, a constant force spring, a belleville spring and a beehive spring. The physiological parameter may be at least one of blood glucose level, blood gas level exposure to an external agent and allergies. The device may further include a reservoir for containing a medicine to be delivered to the person and a fluid transport device, enclosed within the housing, for dispensing medicine from the reservoir to the person, the fluid transport device including a proximal end in fluid communication with the reservoir and a distal end having a penetrating member for piercing the skin of the person to facilitate the delivery of medicine to the person through the fluid transport device. The device may further include a second injection activation device including a second driving mechanism contacting the second fluid transport device for driving the penetrating member from a third position within the housing, through the exit port to the fourth position, external to the housing and into the skin of the person. The sensor device may include means for instructing the second injection activation device to drive the second fluid transport from the third position within the housing, through the exit port to the fourth position, external to the housing and into the skin of the person and to transport an amount of medicine to the person, based on the physiological parameter sensed by the sensor device.

According to yet another embodiment of the invention, an ambulatory medical device includes a transcutaneous member including a penetrating member at a distal end thereof for piercing the skin of the person, a therapeutic element coupled to the transcutaneous member for administering a treatment to a person, a housing containing the therapeutic element and the transcutaneous member, the housing including an exit port for receiving the distal end of the transcutaneous member upon injection of the distal end into the person and means for securing a first wall of the housing to the skin of the person and an injection activation device including a driving mechanism contacting the transcutaneous member for driving the penetrating member from a first position within the housing, through the exit port to a second position, external to the housing and into the skin of the person.

The treatment may be initiated upon the penetrating member being driven into the skin of the person. The ambulatory device may further include a processor for controlling the injection activation device. The therapeutic element may include at least one of pacemaker leads, defibrillator leads, time-release solid-form drugs, magnets, electromagnets, radioactive seeds, thermal elements and one or more transcutaneous electrode nerve stimulus (“TENS”) devices. The driving mechanism of the injection activation device may include a plunger having a body portion extending through an aperture in a second wall of the housing and in frictional contact with the distal end of the transcutaneous member, such that the application of a longitudinal force to the plunger drives the penetrating member from the first position to the second position. The plunger may include a friction member disposed on the body portion, the friction member causing the body portion of the plunger to have a width dimension which is slightly larger than a width dimension of the aperture of the housing, thus requiring a specific longitudinal force to be applied to the plunger to enable the friction member to pass through the aperture, the specific force being translated to the distal end of the transcutaneous member. The friction member may be an annular flange. The plunger further may include a head portion for stopping travel of the plunger by contacting the housing. The plunger may be removable from the housing after the penetrating member is driven to the second position. The driving mechanism of the injection activation device comprises a plunger contained within the housing, the plunger having a first end including a lateral protrusion and a second end in frictional contact with the distal end of the transcutaneous member, the injection activation device further including a biasing spring for biasing the plunger for driving the penetrating member from the first position to the second position, and the lateral protrusion being in contact with an internal ridge of the housing, with the penetrating member in the first position, thereby preventing the plunger from driving the penetrating member from the first position to the second position. The housing may include an actuator for urging the lateral protrusion from the internal ridge, thereby causing the plunger to drive the penetrating member from the first position to the second position. The actuator may include a finger coupled to an inside surface of a flexible wall portion of the housing, a distal end of the finger being in contact with the lateral protrusion such that an application of pressure to the flexible wall portion causes the finger to urge the lateral protrusion from the ridge, thereby causing the plunger to drive the penetrating member from the first position to the second position. The distal end of the finger, upon the application of pressure to the flexible wall portion, may move in same the direction as the flexible wall portion. The distal end of the finger, upon the application of pressure to the flexible wall portion, may move in a substantially opposite direction as the flexible wall portion. The finger may include a pivot which causes the distal end of the finger to move in a direction substantially opposite that of the flexible wall portion. The driving mechanism of the injection activation device may include a pivoting arm and the injection activation device further includes a latch assembly, the pivoting arm having a proximal end pivotally coupled to an inside surface of a wall of the housing and a distal end in contact with the latch assembly integral with a side wall of the housing, the transcutaneous member being coupled to the arm such that when the distal end of the arm is in contact with the latch assembly, the penetrating member is in the first position. The injection activation device further includes a biasing spring attached between the proximal and distal ends of the arm and a wall of the housing, the biasing spring urging the arm to drive the penetrating member to the second position and the latch assembly includes a latch for contacting the distal end of the pivoting arm to prevent the pivoting arm from driving the penetrating member from the first position to the second position under the influence of the biasing spring and a latch release mechanism for moving the latch out of contact with the distal end of the pivoting arm, thereby enabling the pivoting arm to drive the penetrating member from the first position to the second position under the influence of the biasing spring. The latch release mechanism may include an electrically driven actuator coupled between the latch and the side wall of the housing, such that, upon the application of a charge to the electrically driven actuator, the electrically driven actuator activates to pull the latch out of contact with the distal end of the pivoting arm. The latch release mechanism may include a mechanical lever coupled to the latch and protruding through the side wall, such that, upon the lever being pulled away from the housing, the latch is pulled out of contact with the distal end of the pivoting arm. The injection activation device may include a discrete secondary housing, the plunger including a first end having a lateral protrusion and a second end in frictional contact with the distal end of the transcutaneous member, the second end of the plunger extending from within the secondary housing, out of a distal end thereof into the aperture of the housing and into frictional contact with the distal end of the transcutaneous member. The injection activation device may further include a biasing spring coupled between the first end of the plunger and a proximal end of the secondary housing within the secondary housing for biasing the plunger for driving the penetrating member from the first position to the second position, the lateral protrusion being in contact with an internal ridge of the secondary housing, with the penetrating member in the first position, thereby preventing the plunger from driving the penetrating member from the first position to the second position. The secondary housing including an actuator for urging the lateral protrusion from the internal ridge, thereby causing the plunger to drive the penetrating member from the first position to the second position. The injection activation device may include a discrete secondary housing, the plunger including a first end having a lateral protrusion and a second end in frictional contact with the distal end of the transcutaneous member, the second end of the plunger extending from within the secondary housing, out of a distal end thereof into the aperture of the housing and into frictional contact with the distal end of the transcutaneous member. The injection activation device may further include a biasing spring coupled between the first end of the plunger and a proximal end of the secondary housing within the secondary housing for biasing the plunger for driving the penetrating member from the first position to the second position, the lateral protrusion being in contact with a latch assembly of the secondary housing, with the penetrating member in the first position, thereby preventing the plunger from driving the penetrating member from the first position to the second position. The latch assembly may include a latch for contacting the lateral protrusion of the plunger to prevent the plunger from driving the penetrating member from the first position to the second position under the influence of the biasing spring and a latch release mechanism coupled to the housing for moving the latch out of contact with the lateral protrusion, thereby enabling the plunger to drive the penetrating member from the first position to the second position under the influence of the biasing spring, the latch The release mechanism may include an electrically driven actuator coupled between the latch and the side wall of the housing, such that, upon the application of a charge to the electrically driven actuator, the electrically driven actuator activates to pull the latch out of contact with the distal end of the pivoting arm. The driving mechanism comprising a plunger having a first end in frictional contact with the distal end of the transcutaneous member, the plunger being biased to drive the penetrating member from the first position to the second position, the injection activation device further comprising a latch for contacting the plunger to maintain the penetrating member in the first position, the latch including an electrically driven actuator coupled to the latch, such that, upon the application of a charge to the electrically driven actuator, the electrically driven actuator activates to pull the latch out of contact with the plunger, thereby enabling the plunger to drive the penetrating means from the first position to the second position.

According to yet another embodiment of the invention, a device for monitoring a parameter of a person includes a sensor device for measuring a parameter associated with the person, a processor for processing measurements of the parameter generated by the sensor device, a first transcutaneous member coupled to the sensor device and the processor, including a first penetrating member at a distal end thereof for piercing the skin of the person, a reservoir for containing a medicine to be delivered to the person, a fluid transport device for dispensing medicine from the reservoir to the person, the fluid transport device including a second transcutaneous member including a proximal end in fluid communication with the reservoir and a distal end having a second penetrating member for piercing the skin of the person to facilitate the delivery of medicine to the person through the fluid transport device, a housing containing the sensor device, the first transcutaneous member, the reservoir, the fluid transport device and the processor, the housing including an exit ports for receiving the distal ends of the first and second transcutaneous members upon injection of the distal ends into the person and means for securing a first wall of the housing to the skin of the person, a first injection activation device including a driving mechanism contacting the first transcutaneous member for driving the first penetrating member from a first position within the housing, through the exit port to a second position, external to the housing and into the skin of the person and a second injection activation device including a second driving mechanism contacting the second transcutaneous member for driving the second penetrating member from the first position within the housing, through the exit port to the second position, external to the housing and into the skin of the person.

The processor may include an injection activation instruction generation portion for providing injection activation instructions to the first and second injection activation devices based on a trigger signal provided to the injection activation instruction generation portion. The trigger signal may be generated within the processor based on timing instructions programmed into the processor, the timing instructions causing the trigger signal to be provided to the injection activation instruction generation portion at predetermined time intervals. The trigger signal may be generated within the processor based on a sensor input to the processor from a second sensor which monitors at least one environmental parameter, the sensor input causing the trigger signal to be provided to the injection activation instruction generation portion upon the environmental parameter reaching a predetermined level. The second sensor may be disposed within the housing or located externally from the housing. The second sensor may include a transmitter for transmitting the sensor input to a receiver associated with the processor. The environmental parameter may include at least one of temperature, pressure, oxygen level, light and the presence of a chemical agent. The processor may provide injection activation instructions to the first injection activation device when the second sensor determines that the at least one environmental parameter has reached the predetermined level. The processor may provide injection activation instructions to the second injection activation device when the second sensor determines that the at least one environmental parameter has reached the predetermined level. The sensor device may monitor a physiological parameter associated with the person and the processor may provide first injection activation instructions to the first injection activation device when the second sensor determines that the at least one environmental parameter has reaches the predetermined level and provides second injection activation instructions to the second injection activation device when the sensor device determines that the physiological parameter has reached a predetermined level.

### BRIEF DESCRIPTION OF THE DRAWINGS

FIG. 1 is a perspective view of a first exemplary embodiment of a physiological parameter sensing device constructed in accordance with the present invention and shown secured on a patient, and a remote control device for use with the physiological parameter sensing device (the remote control device being enlarged with respect to the patient and the physiological parameter sensing device for purposes of illustration);

FIG. 2 is a sectional view of the physiological parameter sensing device of FIG. 1, with a slidably movable penetrating member shown deploying a subcutaneous cannula;

FIG. 3 is cutaway view of another embodiment of a physiological parameter sensing device in accordance with the present invention;

FIG. 4 is cutaway view of another embodiment of a physiological parameter sensing device in accordance with the present invention;

FIGS. 5A and 5B are various views of another embodiment of a physiological parameter sensing device in accordance with the present invention;

FIGS. 6A-6C are various views of one embodiment of a physiological parameter sensing device in accordance with the present invention;

FIGS. 7A-7D are various views of another embodiment of a physiological parameter sensing device in accordance with the present invention;

FIGS. 8A-8B are various views of another embodiment of a physiological parameter sensing device in accordance with the present invention;

FIG. 9 is a cutaway view of another embodiment of a physiological parameter sensing device in accordance with the present invention;

FIGS. 10A-10D are various views of another embodiment of a physiological parameter sensing device in accordance with the present invention;

FIGS. 11A-11E are various views of another embodiment of a physiological parameter sensing device in accordance with the present invention;

FIGS. 12A-12C are various views of another embodiment of a physiological parameter sensing device in accordance with the present invention;

FIG. 13 is a cutaway view of another embodiment of a physiological parameter sensing device in accordance with the present invention;

FIGS. 14A-14C are various views of another embodiment of a physiological parameter sensing device in accordance with the present invention;

FIGS. 15A-15B are various views of another embodiment of a physiological parameter sensing device in accordance with the present invention;

FIGS. 16A-16C are various views of another embodiment of a physiological parameter sensing device in accordance with the present invention;

FIGS. 17A- 17D are various views of another embodiment of a physiological parameter sensing device in accordance with the present invention;

FIG. 18 is a perspective view of another embodiment of a physiological parameter sensing device in accordance with the present invention;

FIG. 19 is a perspective view of another embodiment of a physiological parameter sensing device in accordance with the present invention;

FIGS. 20A-20B are various views of another embodiment of a physiological parameter sensing device in accordance with the present invention;

FIGS. 21A-21C are various views of another embodiment of a physiological parameter sensing device in accordance with the present invention;

FIG. 22 is a cutaway view of another embodiment of a physiological parameter sensing device in accordance with the present invention;

FIG. 23 is a perspective view of another embodiment of a physiological parameter sensing device in accordance with the present invention;

FIGS. 24A-24D are various views of another embodiment of a physiological parameter sensing device in accordance with the present invention;

FIGS. 25A-25C are various views of another embodiment of a physiological parameter sensing device in accordance with the present invention;

FIGS. 26A-26H are various views of another embodiment of a physiological parameter sensing device in accordance with the present invention;

FIG. 27 is a cutaway view of another embodiment of a physiological parameter sensing device in accordance with the present invention;

FIGS. 28A-28D are various views of another embodiment of a physiological parameter sensing device in accordance with the present invention;

FIGS. 29A-29E are various views of another embodiment of a physiological parameter sensing device in accordance with the present invention;

FIGS. 30A-30D are various views of another embodiment of a physiological parameter sensing device in accordance with the present invention;

FIG. 31 is a perspective view of another embodiment of a physiological parameter sensing device in accordance with the present invention;

FIG. 32 is a perspective view of another embodiment of a physiological parameter sensing device in accordance with the present invention;

FIG. 33 is a cutaway view of another embodiment of a physiological parameter sensing device in accordance with the present invention;

FIG. 34 is a cutaway view of another embodiment of a physiological parameter sensing device in accordance with the present invention;

FIG. 35 is a cutaway view of another embodiment of a physiological parameter sensing device in accordance with the present invention;

FIG. 36 is a cutaway view of another embodiment of a physiological parameter sensing device in accordance with the present invention;

FIG. 37 is a cutaway view of another embodiment of a physiological parameter sensing device in accordance with the present invention;

FIGS. 38A-38B are various views of another embodiment of a physiological parameter sensing device in accordance with the present invention;

FIGS. 39A-39C are various views of another embodiment of a physiological parameter sensing device in accordance with the present invention; and

FIG. 40 is a cutaway view of a therapeutic medical device in accordance with the present invention.

### DETAILED DESCRIPTION

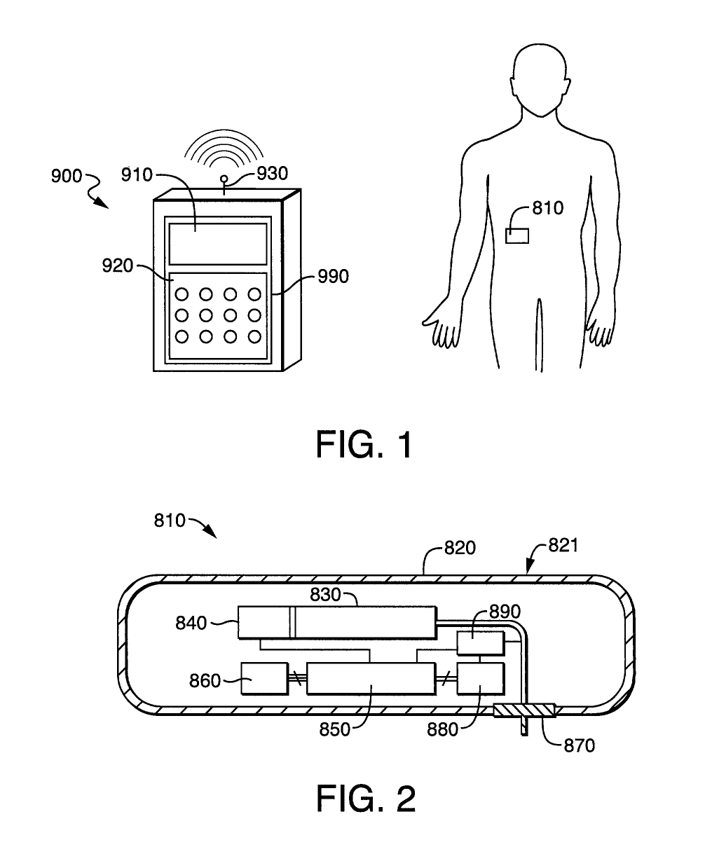

Referring to FIGS. 1 and 2, there is illustrated the various embodiments of a physiologic sensing device constructed in accordance with the present invention. Preferably, the sensing device includes a physiologic sensor disposed at the tip of a transcutaneous member which is injected into the skin of the person wearing the device types of fluids that can be sampled and withdrawn by the device of the present invention include blood, interstitial fluid and other bodily fluids. The types of medical conditions that the fluid delivery device of the present invention might be used to treat include, but are not limited to, diabetes, cardiovascular disease, pain, chronic pain, cancer, AIDS, neurological diseases, Alzheimer's disease, ALS, hepatitis, Parkinson's disease or spasticity. The physiological conditions that can be sampled by the device of the present invention include, but are not limited to, blood glucose, blood gas, exposure to external elements, allergic reactions and body temperature. Optionally, the device may include a fluid withdrawal device for facilitating the sensing of the physiologic conditions within a housing of the device and a fluid infusion device for delivering medicinal fluid to the person, based on the sensing of the physiologic condition.

Referring to FIG. 2, the device 810 generally includes an exit port assembly 870 including a transcutaneous patient access tool, a sensor assembly 830, and a processor or electronic microcontroller (hereinafter referred to as the “local” processor) 850 connected to the dispenser 840.

The local processor 850 is programmed to cause sensor assembly 830 to be deployed based on instructions from a separate, remote control device 900, an example of which is shown in FIG. 1. Referring also to FIG. 2, the device 810 further includes a wireless receiver 860 connected to the local processor 850 for receiving the instructions from the separate, remote control device 900 and delivering the instructions to the local processor. The device 810 also includes a housing 820 containing the exit port assembly 870, the sensor assembly 840, the local processor 850, and the wireless receiver 860.

As shown, the housing 820 is free of user input components for providing instructions to the local processor 850, such as electromechanical switches or buttons on an outer surface 821 of the housing, or interfaces otherwise accessible to a user to adjust the programmed flow rate through the local processor 850. The lack of user input components allows the size, complexity and costs of the sensor device 810 to be substantially reduced so that the sensor device 810 lends itself to being small and disposable in nature.

In order to program, adjust the programming of, or otherwise communicate user inputs to the local processor 850, the sensor device 810 includes the wireless communication element, or receiver 860 for receiving the user inputs from the separate, remote control device 900 of FIG. 1. Signals can be sent via a communication element (not shown) of the remote control device 900, which can include or be connected to an antenna 930, shown in FIG. 1 as being external to the device 900.

Referring to FIGS. 1 and 2, the remote control device 900 has user input components, including an array of electromechanical switches, such as the membrane keypad 920 shown. The control device 900 also includes user output components, including a visual display, such as a liquid crystal display (LCD) 910. Alternatively, the control device can be provided with a touch screen for both user input and output. Although not shown in FIG. 1, the remote control device 900 has its own processor (hereinafter referred to as the “remote” processor) connected to the membrane keypad 920 and the LCD 910. The remote processor receives the user inputs from the membrane keypad 920 and provides “flow” instructions for transmission to the sensor device 810, and provides information to the LCD 910. Since the remote control device 900 also includes a visual display 910, the sensor device 810 can be void of an information screen, further reducing the size, complexity and costs of the sensor device 810.

The communication element 860 of the sensor device 810 preferably receives electronic communication from the remote control device 900 using radio frequency or other wireless communication standards and protocols. In a preferred embodiment, the communication element 860 is a two-way communication element, including a receiver and a transmitter, for allowing the fluid delivery device 810 to send information back to the remote control device 900. In such an embodiment, the remote control device 900 also includes an integral communication element 860 comprising a receiver and a transmitter, for allowing the remote control device 900 to receive the information sent by the sensor device 810. Specific instructions communicated to the sensor device 810 include a time schedule for taking samples, as described below and specific levels of a physiological condition of the person that warrant either a warning or an infusion of medicine, or both. Alternatively, the sensor device 810 may include a user interface, including various user input and information displaying components built into the housing 820, thus providing a unitary sensing device which does not require the use of a separate remote control device.

The local processor 850 of the device 810 contains all the computer programs and electronic circuitry needed to allow a user to program the desired flow patterns and adjust the program as necessary. Such circuitry can include one or more microprocessors, digital and analog integrated circuits, resistors, capacitors, transistors and other semiconductors and other electronic components known to those skilled in the art. The local processor 850 also includes programming, electronic circuitry and memory to properly activate the sensor assembly 890 at the needed time intervals.

In the exemplary embodiment of FIG. 2, the sensor device 810 includes a power supply 880, such as a battery or capacitor, for supplying power to the local processor 850. The power supply 880 is preferably integrated into the fluid delivery device 810, but can be provided as replaceable, e.g., a replaceable battery.

Although not shown, the device 810 can also be provided with an adhesive layer on the outer surface of the housing 820 for securing the device 810 directly to the skin of a patient. The adhesive layer is preferably provided in a continuous ring encircling the exit port assembly 870 in order to provide a protective seal around the penetrated skin. The housing 820 can be made from flexible material, or can be provided with flexible hinged sections that allow the fluid delivery device 810 to flex during patient movement to prevent detachment and aid in patient comfort.

Accordingly, the device 810 can be used to sense or measure physiological conditions in situ, withdraw fluids from the user in order to sample the fluids and, optionally, to control the delivery of the medicinal fluid to the user based on the samples taken. For this purpose, sensor assembly 890 is capable of receiving fluids withdrawn from the user and sensing various physiological conditions of the user.

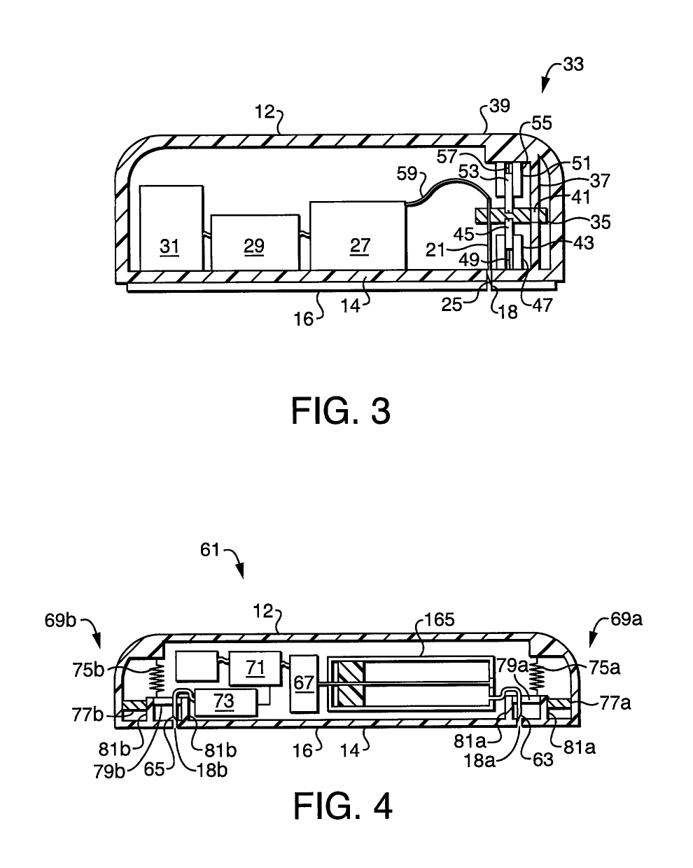

Referring now to FIG. 3, a first embodiment 8 of the present invention includes a housing 12 for containing a sensor assembly and other control devices. This embodiment is directed to a device for temporarily implanting a physiological sensor into a patient for monitoring physiological conditions of the patient. The footprint of the housing 12 may be square, rectangular, oval or other geometry, depending on the size requirements for containing the sensing and control elements as well as the comfort requirements of the user. Housing 12 includes a first wall 14 having, preferably, an adhesive material 16 attached thereto for enabling the housing 12 to be securely adhered to the skin of the patient. While, in the preferred embodiment, the attachment means, as shown in FIG. 3, is an adhesive tape attached to the first wall 14 of the housing 12, it will be understood that any means for securing the housing 12 to the patient, such as simply taping the housing 12 to the skin of the patient, or securing the housing to the patient by means of a strap or other similar device.

Housing 12 further includes an exit port 18, disposed in the first wall 14, for enabling transcutaneous member 21 which, in this embodiment, is in the form of a rigid needle having a penetrating portion 25, such as a sharpened point of the member 21 for penetrating the skin of the patient upon deployment of the member as described below. Also contained within housing 12 is a sensor assembly 27 for analyzing fluids contacted or withdrawn by the transcutaneous member 21, electronics 29, including a local processor for providing operating instruction to the sensor assembly 27 and for interacting with an associated remote control device and a battery 31. An actuator portion, generally indicated at 33, operates to drive the transcutaneous member 21 into the skin of the patient and to withdraw the member 21 from the skin of the patient.

Actuator portion 33 includes a slider 35 which is fixed to the member 21 at one end thereof and which is slidably mounted on an alignment rod 37 which is rigidly fixed within the housing 12 between first wall 14 and a second wall 39. Slider 34 includes an aperture 41 through which the alignment rod 37 is disposed. The engagement of the slider 35 with the alignment rod 37 prevents the slider 35 from rotating or moving out of alignment during the insertion or withdrawal of the transcutaneous member 21.

Actuator portion 33 further includes insertion device 43 which includes an insertion plunger 45 coupled to slider 35 at a first end thereof and having a body portion slidably disposed within guide portion 47. An insertion actuator 49 is coupled between a second end of the insertion plunger 45 and the first wall 14. Actuator portion 33 further includes withdrawal device 51 which includes a withdrawal plunger 53 coupled to slider 35 at a first end thereof and having a body portion slidably disposed within guide portion 55. A withdrawal actuator 57 is coupled between a second end of the withdrawal plunger 53 and the second wall 39. In the preferred embodiment, insertion actuator 43 and withdrawal actuator 51 each include a shape memory alloy or polymer which contracts under the influence of an electrical charge. However, other devices may be utilized for the insertion actuator 43 and withdrawal actuator 51, such as a piezo electric actuator and a solenoid. Accordingly, upon receipt of respective instructions from local processor 29, the transcutaneous member 21 may be activated to be inserted into the skin of the patient or withdrawn from the skin of the patient. Specifically, upon receipt of insertion instructions from the remote control device, local processor 29 sends an electrical charge to the insertion actuator 43, which causes it to contract, thereby pulling the slider 35 and consequently the transcutaneous member 21 toward first wall 14, resulting in the member 21 being pulled through exit port 18 and into the skin of the patient. Likewise, upon receipt of withdrawal instructions from the remote control device, local processor 29 sends an electrical charge to the withdrawal actuator 57, which causes it to contract, thereby pulling the slider 35 and consequently the transcutaneous member 21 toward second wall 39, resulting in the member 21 being pulled back through exit port 18 and into the housing 12.

In a first version of this embodiment, transcutaneous member 21 includes a physiologic sensor (not shown) disposed at or near the penetrating portion 25, such that, upon the insertion of the member 21 into the skin of the patient, the physiological sensor is inserted into the patient. The physiological sensor may be any type of sensor known in the art and may be used to monitor any physiological condition, including, but not limited to, blood glucose levels, blood gas levels and exposure to external elements. In this embodiment, the implanted sensor takes the necessary measurements within the medium to which it is implanted and transmits, via line 59, the measurements taken by the sensor to the sensor assembly 27 for further processing. The device 8 may then transmit these measurements to the remote control device to update the patient on the physiological condition being monitored.

In a second version of this embodiment, transcutaneous member 21 comprises a hollow cannula and sensor assembly 27 includes a fluid withdrawal mechanism for drawing fluid through the cannula and into the sensor assembly for testing. The physiological sensor or sensors are included in the sensor assembly, and the withdrawn fluid is monitored within the sensor assembly 27.

Since the device 8 includes both insertion device 43 and withdrawal device 51, the transcutaneous member may be programmed to be inserted and withdrawn repeatedly at various times through a monitoring period. For example, in some instances, the device 8 may be worn by the patient for a period of days. In order to monitor a particular physiological condition of the patient at specific times of the day, the device can be programmed to insert the member 21 at a particular time to take a sample and then to withdraw the member 21 after the sample has been taken. This reduces the discomfort for the patient of having the member 21 inserted into the patient's skin for extended periods of time and also insures that the samples being taken at a particular time of day are indeed samples from that time, and not another time while the member 21 was inserted in the patient.

Another embodiment 61 of the present invention is shown in FIG. 4. This embodiment also includes a housing 12 for enclosing the various elements of the device. Housing 12 includes a first wall 14 having, preferably, an adhesive material 16 attached thereto for enabling the housing 12 to be securely attached to the patient. Housing 12 further includes a first exit port 18a, disposed in the first wall 14, for enabling transcutaneous member 63 which, in this embodiment, is in the form of a rigid hollow needle having a penetrating portion, such as a sharpened point of the member 63, to penetrate the skin of the patient upon deployment of the member 63. Housing 12 also includes a second exit port 18b, disposed in the first wall 14, for enabling transcutaneous member 65 to be driven into the skin of the patient from within the housing 12 upon deployment of the member 65.

Associated with transcutaneous member 63 and contained within housing 12 are a cartridge 65 for containing a fluid to be infused into the patient through member 65, a drive mechanism 67 for driving the fluid from the cartridge 165 through the member 63, an injection actuator 69a and a local processor 71 for controlling the operation of the drive mechanism 67 and the injection actuator 69. Associated with transcutaneous member 65 and contained within housing 12 are a sensor assembly 73 and an injection actuator 69b. Sensor assembly 73 operates under controls received from local processor 71.

Injection actuators 69a and 69b comprise latch mechanisms 77a and 77b for maintaining the members 63 and 65, respectively, in the undeployed state. The members 63 and 65 are coupled to sliders 79a and 79b, respectively, which are slidably mounted within walls 81a and 81b. Latch mechanisms 77a and 77b maintain the sliders 79a and 79b in place against the force exerted on the sliders 79a and 79b by compressed springs 75a and 75b. Latch mechanisms 77a and 77b include latches and release devices (not specifically shown in FIG. 4) which, when activated, pull the latches out of contact with the sliders 79a and 79b, thus allowing springs 75a and 75b to release their energy, thus pushing their associated members 63 and 65 through exit ports 18a and 18b, respectively, and into the skin of the patient. In one embodiment of the present invention, the release devices comprise a memory shape alloy or polymer which contracts under the influence of an electrical charge. However, other devices may be utilized for the release devices, such as a piezo electric actuator and a solenoid. Accordingly, upon receipt of respective instructions from local processor 71, either or both of the transcutaneous members 63 and 65 may be activated to be inserted into the skin of the patient. Specifically, upon receipt of insertion instructions from the remote control device, local processor 71 sends an electrical charge to the appropriate release device, which causes it to contract, thereby pulling the latch out of contact with the slider 79a and/or 79b and consequently releasing the transcutaneous member 63 toward first wall 14, through exit port 18a/18b and into the skin of the patient.

Alternatively, local processor 71 may control the injection of either or both of the transcutaneous members 63 and 65 based on certain parameters either detected or monitored by the device 61, rather than instructions received from a remote processor. For example, the local processor 71 may be programmed to inject the sensor transcutaneous member 65 to take readings of physiological parameters at certain time intervals for certain periods of time. Furthermore, the local processor 71 may be programmed to cause the infusion transcutaneous member 63 to be injected into the person for infusion of the fluid stored in the cartridge 165 based on one or more of several factors.

The local processor 71 can be programmed to initiate the injection of the infusion transcutaneous member 63 based on physiological parameters detected by the sensor transcutaneous member 65. In this case, when a physiological parameter being monitored by the sensor transcutaneous member 65 reaches or exceeds a predetermined threshold, the local processor 71 will initiate the injection of infusion transcutaneous member 63, to facilitate the delivery of fluid to the person for the treatment of the condition associated with the physiological parameter being monitored. For example, if the sensor assembly 63 of the device includes a sensor transcutaneous member 65 for monitoring the blood sugar level of the person, and the level is detected to have dropped below a threshold level preprogrammed into the local processor 71, the local processor 71 will initiate the injection of the infusion transcutaneous member 63 into the person, to facilitate the delivery of insulin, which is stored in the cartridge 165, to the person. Other physiological parameters that may be monitored and treated in this manner include blood gas levels, body temperature, allergic reactions, respiration rate, arrhythmia, blood cell count, blood flow rate, average blood clotting time, thrombogenicity, blood oxygen content, blood pH and blood toxicity levels.

Sensor assembly 73 may also include an environmental sensor which is housed within housing 12 for sensing external environmental conditions to which the person is exposed. Upon detection that the environmental condition has reached or exceeded a predetermined threshold, the local processor 71 will initiate the injection of infusion transcutaneous member 63, to facilitate the delivery of fluid to the person for the treatment of the condition associated with the environmental condition being monitored. For example, if the device is worn by a soldier in a combat situation where the use of chemical weapons is possible, the environmental sensor monitors the air to which the soldier is exposed for the presence of certain chemicals. If the sensor detects such chemicals, the local processor 71 will initiate the injection of the infusion transcutaneous member 63 into the soldier, to facilitate the delivery of an antidote for the chemical, which is stored in the cartridge 165, to the soldier. Other environmental parameters that may be monitored and treated in this manner include air temperature, air pressure, the amount of oxygen in the air, the presence (or absence) of light to which the person is exposed and the presence of nuclear or other hazardous waste.

Alternatively, the device may include an external sensor assembly for monitoring environmental conditions, in which the external sensor includes a transmitter for transmitting the detection of the predetermined level of the environmental condition to a local processor included within the housing of the device. This enables a single sensor assembly to be used to monitor an environmental condition and to transmit the detection to multiple people who are each wearing the device of the present invention. The device may also include an audible, visual and/or electronic alarm to alert the wearer of the detection of the predetermined level of the environmental condition.

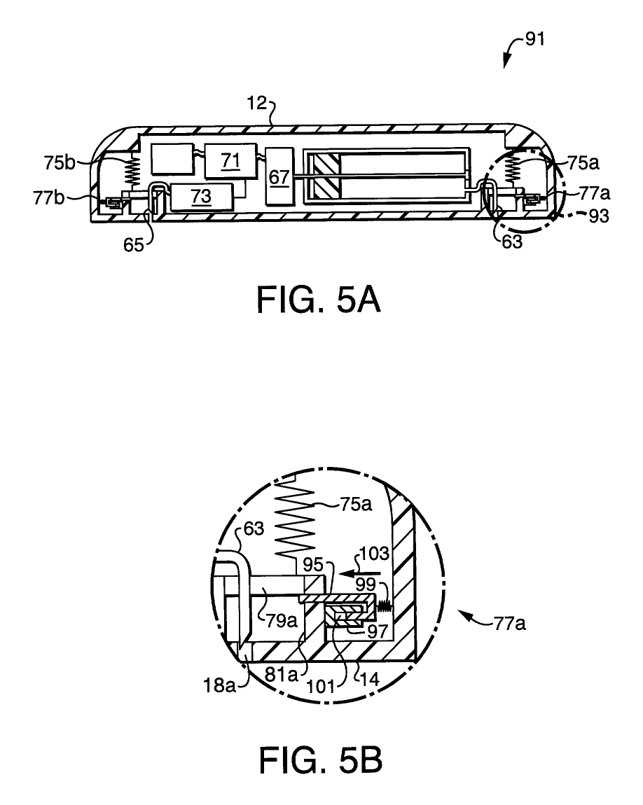

FIG. 5A shows another embodiment 91 of the present invention in which the latch mechanisms 77a and 77b comprise gas driven actuators. Circled portion 93, which includes the latch mechanism 77a, is shown in detail in FIG. 5B. As shown in FIG. 5B, latch mechanism 77a includes a latch arm 95 having a distal end which extends through wall 81a and which maintains slider 79a in the undeployed position against the force of compressed spring 75a. A proximal end of arm 95 is mounted within latch activation member 97. Latch arm 95 is also biased in the direction indicated by arrow 103 to maintain the contact between the distal end of arm 95 and the slider 79a. Latch activation member 97 includes a gas generation chamber 101 which, upon receiving activation instructions from local processor 71, generates a gas within the chamber 101, which causes the proximal end of arm 95 to be pushed in the direction opposite that indicated by arrow 103, thus causing the distal end of arm 95 to be pushed out of contact with slider 79a. This enables spring 75a to release its energy, thereby forcing slider 79a and transcutaneous member 63 through exit port 18a and into the skin of the patient.

Based on the above, the present invention is directed to a device including a transcutaneous member which is injected into the skin of the patient for the purpose of sampling or measuring physiological parameters. In a first embodiment, a sensor device is disposed at the end of the member which is injected into the patient and the sampling operation takes place within the patient. In another embodiment, the transcutaneous member is a hollow cannula through which the fluid being sampled is drawn into a sensor assembly located within the housing of the invention. The sampling operation takes place within the housing. The invention may also include a fluid delivery device housed within the housing which facilitates the delivery of fluids, such as medicines, into the patient. The operation of the fluid delivery device may be coupled to the sensor device such that, when the sensor device detects a predetermined condition within the patient, it can instruct the fluid delivery device to deliver a certain amount of the associated fluid to the patient.

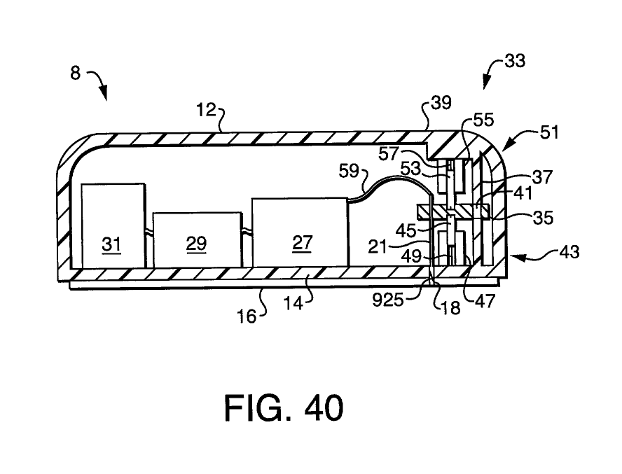

Alternatively, the present invention may be utilized for the purpose of injecting a non-fluidic therapeutic medical device into the patient. In such an embodiment, a therapeutic medical device is disposed at the end of the transcutaneous member, which is then injected into the patient. Some examples of therapeutic medical devices which may be incorporated into the transcutaneous member of the present invention include pacemaker leads, defibrillator leads, time-release solid-form drugs, placed under the skin continuously or intermittently, magnets and/or electromagnets for magnetic therapy, radioactive seeds for brachytherapy, thermal elements and one or more Transcutaneous Electrode Nerve Stimulus (“TENS”) devices for pain control. In the latter application, a plurality of transcutaneous members and injections actuators may be incorporated into the device to facilitate the transcutaneous injection and withdrawal of more than one TENS device. Such a device is shown in FIG. 40, in which a therapeutic medical device 925 is disposed proximate the penetrating member of transcutaneous member 21. Upon injection of the transcutaneous member 21 and therapeutic medical device 925 into the skin of the person, the therapeutic medical device 925 is able to administer the appropriate therapy to the person. The administration of the therapy can be initiated automatically by the injection of the therapeutic medical device 925 into the skin, or the processor 29 can be programmed to control the administration of the therapy. The following describes various embodiments of the invention for facilitating the sensing of various conditions of a patient; the injection of fluids into patient; and the injection of a non-fluidic therapeutic medical device via transcutaneous members housed within a housing which is attached to the skin of the patient. The following embodiments are directed to various injection actuators for injecting and withdrawing the above-described transcutaneous members. Each embodiment may be utilized in connection with either the sensor device or the fluid delivery device.

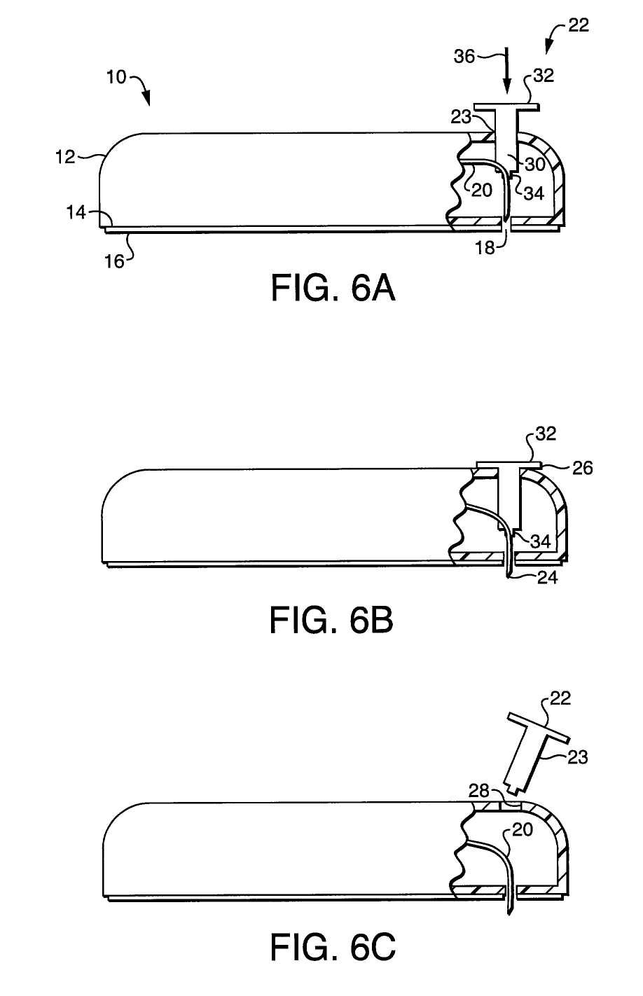

FIGS. 6A-6C show an embodiment including a plunger device 22 having a body portion 30 which extends through an aperture 28 in a second wall of the housing 12, a head portion 32 and a transcutaneous member engagement portion 34 which maintains a frictional engagement with the cannula 20 when the transcutaneous member 20 is in the predeployment stage, or first position, shown in FIG. 6A. Plunger device 22 further includes one or more flanges 23 disposed along the body portion 30 thereof. As shown in FIG. 6A, flanges 23 are initially exterior to the housing 12 in the predeployment stage and cause the plunger device 22 to have a diameter at the point of the flanges 23 which is greater than the diameter of the aperture 28 of the housing 12. After the housing 12 has been attached to the patient, the transcutaneous member is deployed into the skin of the patient by applying manual pressure to the head 32 of the plunger device 22 in the direction shown by arrow 36 of FIG. 6A. Since the flanges 23 cause the body portion 30 to have a larger diameter at the point of the flanges 23 than the diameter of the aperture 28, a specific force is required to compress the flanges to a point where they will pass through the aperture 28. This force, once applied, is great enough to cause the plunger device 22 to force the transcutaneous member through the exit port 18 of the first wall 14 and into the skin of the patient, such as is shown in FIG. 6B.

The head 32 of plunger device 22 is formed such that when the plunger device is in the deployed stage, or second position, such as shown in FIG. 6B, a peripheral edge 26 of the head portion 32 is disposed relative to the housing 12 so as to expose an underside of the head 32 along the edge 26 for facilitating the removal of the plunger device 22 by prying the plunger device 22 away from the housing 12 upon the application of pressure to the underside of the head portion 32. Transcutaneous member engagement portion 34 of the plunger device 22 is constructed to enable the plunger to force the transcutaneous member through the exit port 18 and into the skin of the patient, while allowing the plunger device 22 to be removed from the housing 12 such as is shown in FIG. 6C, and allowing the transcutaneous member 20 to remain in the deployed position shown in FIG. 6C. Once the transcutaneous member 20 is deployed into the skin of the patient, fluid delivery may be commenced.

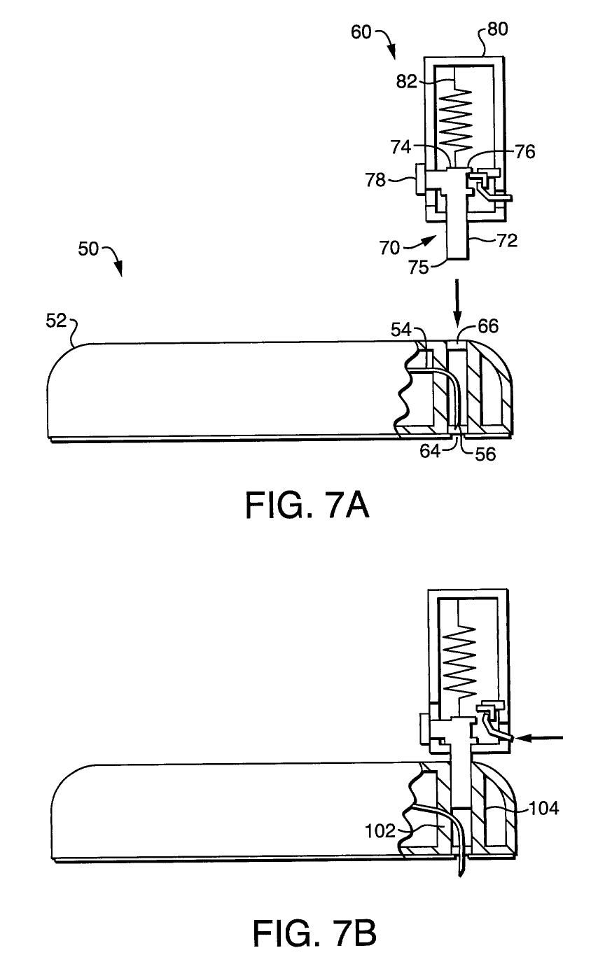

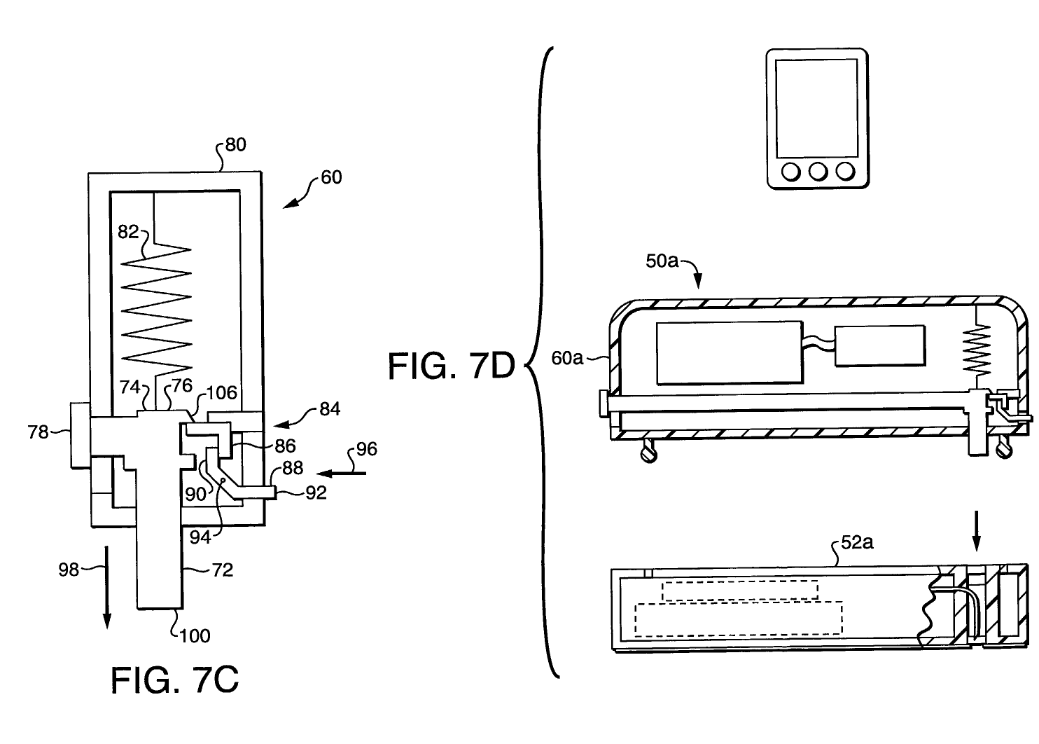

Referring now to FIGS. 7A and 7B, another embodiment 50 of the present invention includes a housing 52 including a transcutaneous member 54 having a penetrating member 56 at a distal end thereof. Fluid delivery device 50 further includes a discrete injection actuator device 60. As shown in FIG. 7A, housing 52 includes an exit port 64 disposed to enable the cannula 54 to be deployed therethrough, and an actuator port 66 disposed opposite the exit port 64. Injection actuator 60 includes a plunger device 70, including a body portion 72, a head portion 74, a cannula engagement portion 75, a lateral protrusion 76 extending from the body portion 72 proximate the head portion 74 and a reset knob 78. Plunger device 70 is contained within a secondary housing 80 along with a spring 82 which is in a compressed state when the plunger device 70 is in the predeployment position shown in FIG. 4A. Referring now to FIG. 7C, which is a more detailed view of the injection actuator 60, the operation of device 50 will be described. As is shown in FIG. 7C, actuator 60 includes a latch mechanism 84 including a latch 86 and a deployment lever 88. Latch 86 is spring biased such that protrusion 76 is in contact with latch 86, thereby preventing the plunger device 70 from deploying. Deployment lever 88 includes a first end 90 in contact with latch 86 and a second end 92 which is external to the housing 80. Deployment lever 94 further includes a pivot point 94 at which it is attached to the housing 80, the pivot point 94 enabling the first end 90 of the lever 88 to move in an opposite direction of the second end 92 of the lever 88 when a force is applied to the second end 92 of lever 88 in the direction of arrow 96. Such a force, when applied to the second end 92 of the lever 88 causes the first end 90 of the lever 88 to move in a direction opposite that shown by arrow 96, causing latch 86 to be driven away from the body portion 72 of the plunger device 70, thereby releasing protrusion 76. Once protrusion 76 is released, energy stored in spring 82 is released, causing plunger 70 to be driven in the direction shown by arrow 98.

Referring back to FIGS. 7A and 7B, prior to deployment, the injection actuator 60 is inserted into aperture 66 of housing 52 such that the transcutaneous member engagement portion 75 of plunger device 70 is in contact with the transcutaneous member 54 while the plunger device 70 is frictionally engaged with side walls 102, 104 of housing 52, thereby holding actuator 60 in place relative to the housing 52. Upon actuating the actuator 60 by applying the force to the second end 92 of lever 88, thereby releasing latch 86 from protrusion 76, plunger device 70 applies a force in the direction of arrow 98 to the transcutaneous member 54, thereby driving the cannula through the exit port 64 into the skin of the patient, as shown in FIG. 7B. At this point, the actuator 60 may be removed from the housing 52 and the reset knob 78 may be pushed in a direction opposite that shown by arrow 98 causing the latch 86 to again engage protrusion 76 with the aid of ramp 106 of protrusion 76, which urges latch 86 away from protrusion 76 while the plunger device 70 is pushed back into the predeployment position shown in FIG. 7C.

FIG. 7D shows an alternative embodiment 50a of the fluid delivery device 50, in which actuator 60a, includes, in addition to the elements described with reference to FIGS. 7A-7C, the fluid delivery device electronics and wireless receiver, which enables the primary housing 52a to have a smaller size and to enable the overall cost of fluid delivery device 50a to be greatly reduced. The actuator 60a is attached to the housing 52a for deployment of the transcutaneous member into the skin of the patient, and can be removed for use with another transcutaneous member injection device. Other disposable and semi-reusable configurations of the multiple housings are disclosed in copending and commonly-owned U.S. Ser. No. 10/081,394, filed Feb. 22, 2002 and entitled MODULAR INFUSION DEVICE AND METHOD.

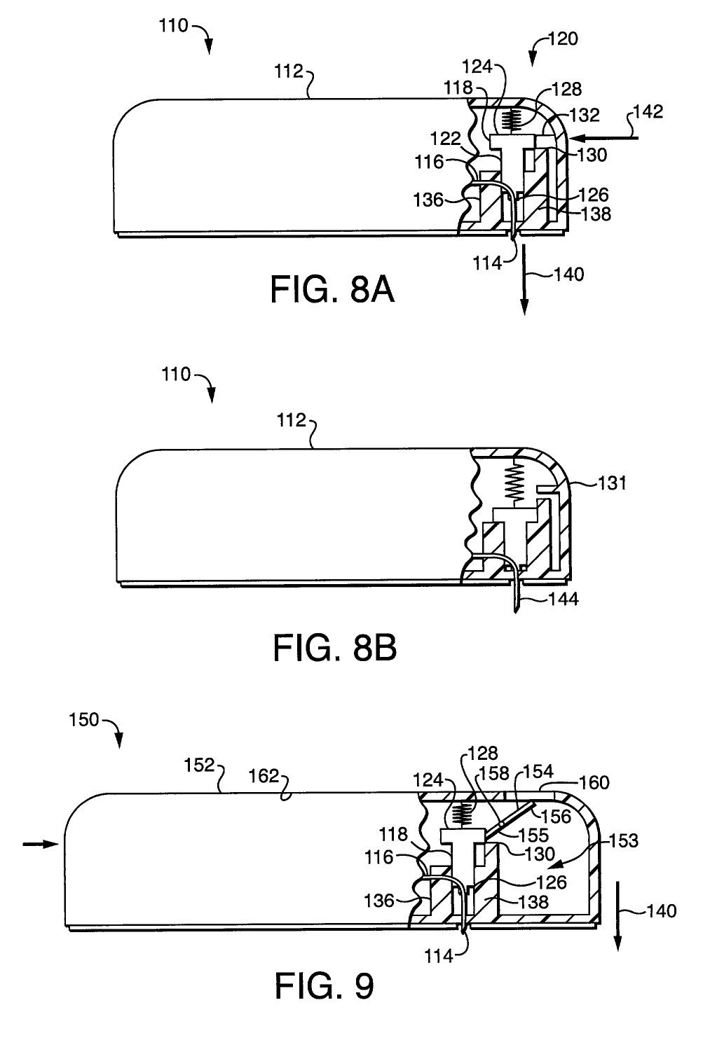

Referring now to FIGS. 8A and 8B, a further embodiment 110 of the present invention will be described. Device 110 includes a housing 112 having an exit port 114 through which transcutaneous member 116 is driven upon actuation of plunger device 118, which is one part of injection actuator 120. Plunger device 118 includes a body portion 122 having a head portion 124 at a first end thereof and a transcutaneous member engagement portion 126 at a second end thereof, the transcutaneous member engagement portion 126 being frictionally engaged with cannula 116 when the actuator 120 is in the predeployment stage shown in FIG. 8A. Actuator 120 further includes a bias spring coupled between the head portion 124 of plunger device 118 and a wall of the housing 112 opposite the head portion 124. As shown in the figures, plunger device 118 is frictionally engaged between walls 136 and 138 of actuator 120. Wall 138 includes a protrusion 130 which engages head portion 124 of plunger device 118 so as to prevent plunger device 118 from being driven in the direction shown by arrow 140 under the force of spring 128. Actuator 120 further includes an urging device 132 extending inwardly from a wall of the housing 112 and in contact with the head portion 124 of plunger device 118.

In this embodiment, at least the wall portion 131 of housing 112 proximate urging device 132 is constructed of a deformable material, such that upon the application of a force to the wall portion 131 to which the urging device 132 is coupled, the force being in the direction shown by arrow 142, urging device 132 applies a similar force in the direction of arrow 142 to the head portion 124 of plunger device 118, thereby urging the head portion 124 away from the protrusion 130 and enabling spring 128 to deenergize, thereby driving the plunger device 118 and the transcutaneous member 116 in the direction shown by arrow 140, causing the penetrating member 144 to be driven into the skin of the patient as shown in FIG. 8B.

FIG. 9 shows a further embodiment 150 of the present invention. Device 150 includes a housing 152 and actuator 153, which is similar to the actuator 120 described with reference to FIGS. 8A and 8B. Accordingly, elements of actuator 153 which are the same as elements of actuator 120 will be described using the same reference numerals used in FIGS. 8A and 8B. As shown in FIG. 9, actuator 153 includes plunger device 118 including a head portion 124 and a transcutaneous member engagement portion 126. Plunger device 118 is frictionally engaged between walls 136 and 138, and wall 138 includes protrusion 130 which engages head portion 124 of plunger device 118 to prevent plunger device 118 from being driven in the direction shown by arrow 140 by biasing spring 128 which, as shown in FIG. 9, is in its compressed, energized state. Actuator 153 includes a lever 154 having a first end 155 in contact with the head portion 124 of plunger device 118 and a second end 156 which is in contact with a deformable portion 160 of wall 162 of housing 152. Lever 154 is pivotally attached to the housing 152 at a pivot point 158, such that when a force is applied to deformable portion 160 of housing 152 in the direction shown by arrow 140, first end 155 of lever 154 urges head portion 124 of plunger device 118 away from protrusion 130 of wall 138, thereby enabling biasing spring 128 to drive plunger device 118 in the direction of arrow 140, thereby driving the transcutaneous member 116 through exit port 114 and into the skin of the patient.

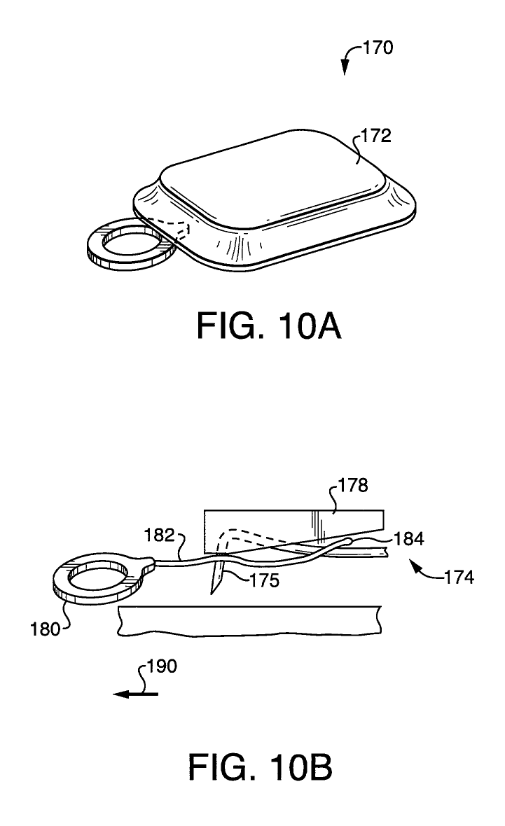

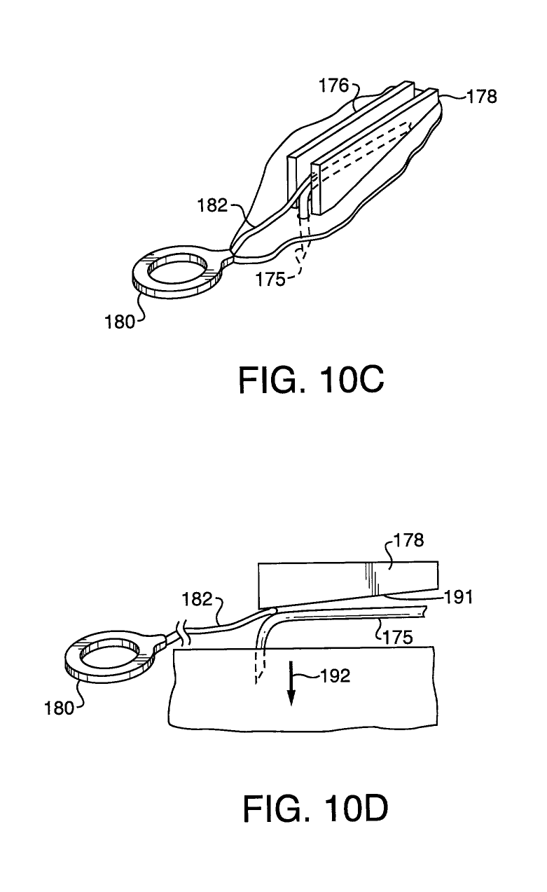

FIG. 10A shows another embodiment 170 of the present invention including a housing 172 and an injection actuator 174 shown in FIG. 10B. As shown in the figures, fluid delivery device 170 includes a transcutaneous member 175 which is disposed between two walls 176 and 178 of housing 172. Injection actuator 174 includes a pull tab 180 which is coupled to an urging device 184 by a connection element 182. Urging device 184 has a width which is wider than the distance between walls 176 and 178, thereby preventing urging device 184 from entering or becoming lodged between walls 176 and 178. When pull tab 180 is pulled in the direction of the arrow shown at 190, connection device 182 pulls urging device 184 along the outer ramped portion 191 of walls 176 and 178, causing the transcutaneous member 175, which initially rides between the walls 176 and 178, to be driven in the direction shown by arrow 192, FIG. 10D, through the exit port (not shown) and into the skin of the patient.

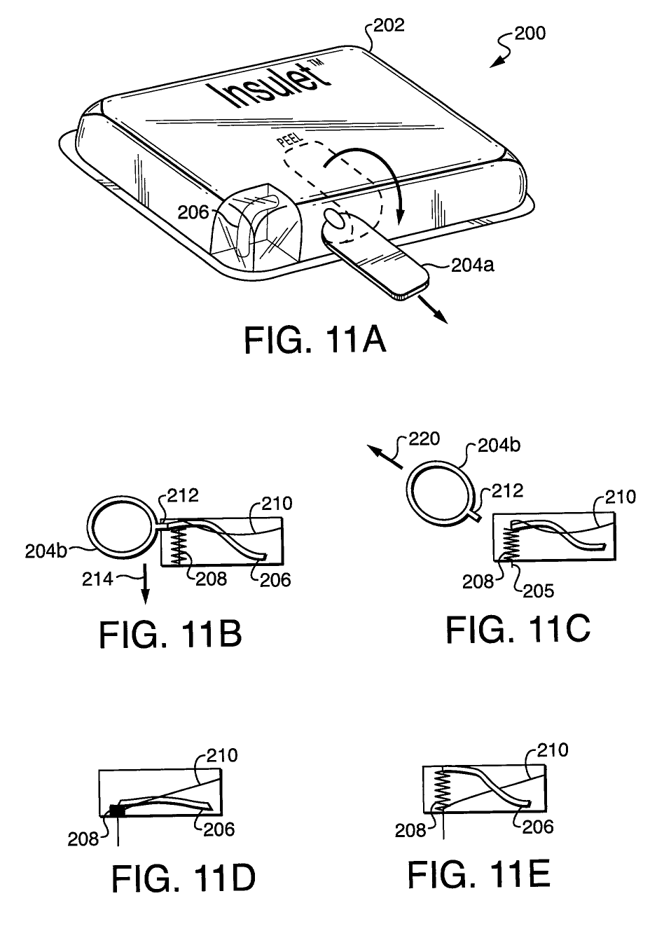

FIG. 11A-11E show yet another embodiment 200 of the device in accordance with the present invention. Device 200 includes a housing 202 and a pull tab which is shown as a flat strip 204a in FIG. 11A and as a ring in 204b in FIG. 11B. It will be understood that any type of pull tab may be used in connection with the current invention in order to deploy the cannula as described herein. Device 200 further includes a cannula 206 having a distal end including a penetrating member for piercing the skin of the patient upon activation of the device 200, a coil compression spring 208, which biases the transcutaneous member 206 in the position shown in FIG. 11B and a leaf spring 210 which is affixed to the housing at a first end and which has a second end in contact with the transcutancous member 206, the leaf spring 210 being biased to apply a force to the transcutancous member 206 in the direction of arrow 214. Pull tab 204B includes an extension member 212 which, as shown in FIG. 11B in its initial state holds the leaf spring 210 in the position shown in FIG. 11B thereby maintaining transcutaneous member 206 in its first position shown under the bias force of spring 208. In order to activate the injection of the cannula into the skin of the patient, pull tab 204B is pulled in the direction indicated by arrow 220, causing extension member 212 to release leaf spring 210, causing the leaf spring to release its energy and drive the transcutaneous member in the direction of arrow 214 resulting in the penetrating member 205 of transcutaneous member 206 being driven into the skin of the patient. Leaf spring 210 has a biasing force which is greater than the biasing force of coil spring 208 such that leaf spring 210 is able to drive the transcutaneous member 206 in the direction of arrow 214 while compressing spring 208. As shown in FIG. 11D, when transcutaneous member 206 is fully inserted into the skin of the patient, coil spring 208 is fully compressed. At this point, leaf spring 210 reaches the end of its travel and, because the length of the leaf spring 210 is less than the distance between the first end of the leaf spring and the former connection point between the second end of the leaf spring and the, the leaf spring to loses contact with the transcutaneous member 206. The release of the transcutaneous member 206 by leaf spring 210 causes spring 208 to release its energy resulting in the transcutaneous member 206 being driven in a direction opposite arrow 214 back to the first position. This embodiment is useful in applications which will be described in further detail below in which a soft flexible transcutaneous member is disposed about the rigid transcutaneous member 206 such that when the rigid transcutaneous member 206 is forced back into its first position by coil spring 208, the flexible transcutaneous member remains within the skin of the patient.

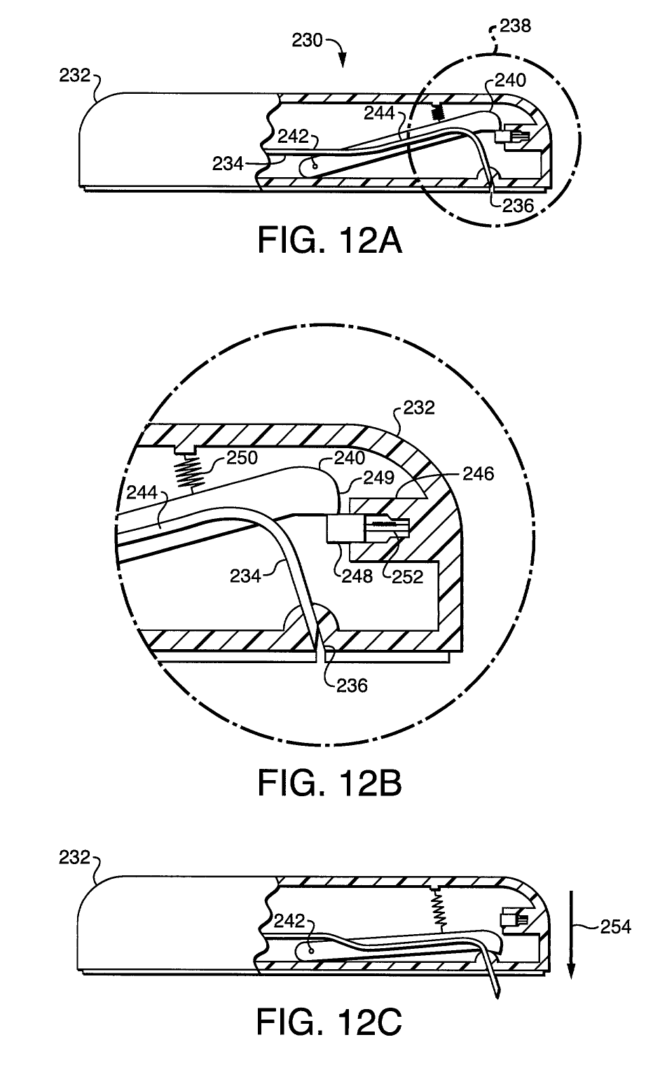

Referring now to FIG. 12A-12C, a further embodiment 230 of the present invention will be described. Device 230 includes a housing 232 having an exit port 236. Transcutaneous member 234 is enclosed within the housing 232 in the first position shown in FIG. 12A and in the inset 238 shown in FIG. 12B. Device 230 further includes a rod 240 which is attached to the housing 232 at a pivot point 242 and which is attached to the transcutaneous member 234 along its length at 244. An injection actuation device includes a latch mechanism 246 having a latch 248 which contacts the end 249 of rod 240 for maintaining the rod 240 in the first position shown in FIG. 12A. A biasing spring is coupled between rod 240 and the housing 232. Biasing spring 250 is in a compressed, energized state when the rod 240 is in the first position, and thus forces the rod against latch 248. Latch mechanism 246 further includes an electrically driven latch actuator 252 which, upon the application of an electrical charge to the latch actuator 252, causes the latch 248 to be moved away from end 249 of rod 240, resulting in the rod 240 and cannula 234 being driven in the direction of arrow 254 under the biasing force of spring 250 to the second position shown in FIG. 12C. Latch actuator 252 receives the electrical charge based on command signals from the local processor, preferably initiated by instructions from the remote processor as described above. In the preferred embodiment, latch actuator 252 is a shape memory alloy or polymer which contracts under the influence of an electrical charge. However, other devices may be utilized for the latch actuator 252, such as a piezo electric actuator and a solenoid.

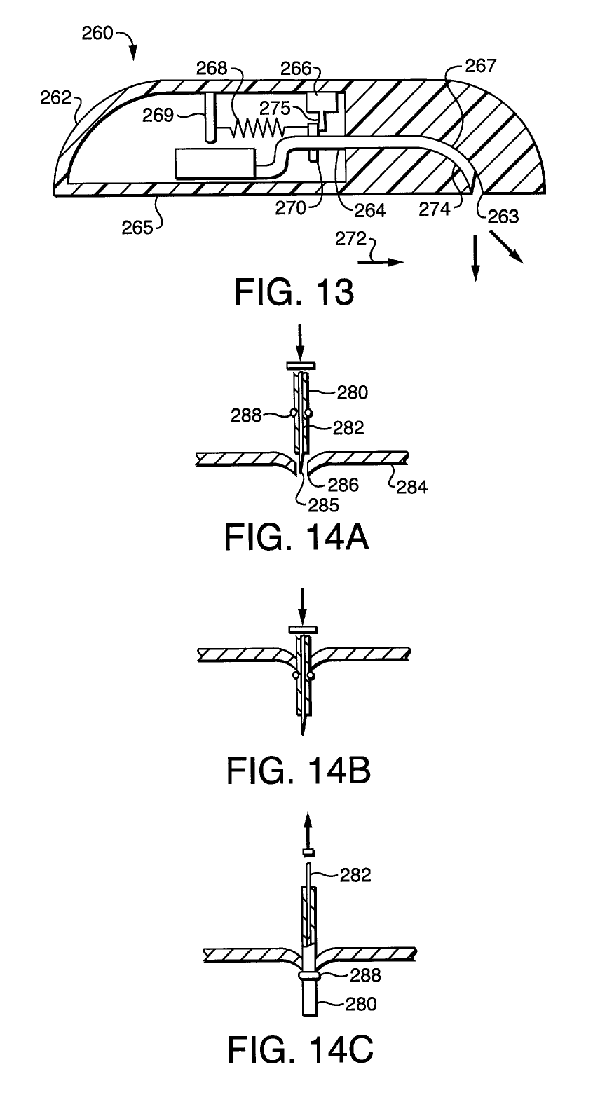

FIG. 13 shows another embodiment 262 of the present invention. Device 260 includes a housing 262, exit port 263 and cannula 264. In this embodiment, transcutaneous member 264 is constructed of a semi-rigid material which enables it to flex as it id driven from the housing 263. Housing 262 includes a transcutaneous member guide portion 267 which deflects the transcutaneous member 264 from the orientation shown with respect to the housing 262 by approximately 15 to 90 degrees as the transcutaneous member 264 passes through the exit port 263. As shown in FIG. 13, the main body portion of the transcutaneous member 264 is disposed substantially parallel to the first wall 265 of the housing 262. Device 260 further includes a latch assembly 266 including a latch 275 and a biasing spring 268 coupled between a first protrusion 269 of housing 262 and a flange 270 of transcutaneous member 264. In the predeployment state shown in FIG. 13, biasing spring 268 is in a compressed, energized state, which maintains the flange 270 of transcutaneous member 264 in contact with the latch 275. Latch assembly 266 may include a manual activation device, such as described with reference to FIG. 7A, or an electrical activation device, such as described with reference to FIG. 12A. In either case, upon activation of the latch mechanism 266, latch 275 is moved out of contact with the flange 270, causing biasing spring 268 to release its energy and drive transcutaneous member 264 through exit port 263 and into the skin of the patient. As the biasing spring 268 is deenergized, the main body portion of the transcutaneous member 264 travels in the direction indicated by arrow 272, while distal end 274 of the transcutaneous member is directed toward first wall 265 by transcutaneous member guide portion 267 of housing 262. As set forth above, transcutaneous member guide portion 267 translates the substantially parallel (to first wall 265) motion of transcutaneous member 264 to a direction approximately 15 to 90 degrees relative to the parallel motion to cause the distal end 274 of transcutaneous member 264 to be directed out of the housing 262 through exit port 263. While the transcutaneous member guide portion 267 of FIG. 10 is shown as a curved channel for deflecting the cannula while guiding it out of the housing 260, it will be understood that it could be in the form of one or more angled planar deflecting surfaces or any suitable combination of guiding components. Furthermore, while, in the preferred embodiment, the transcutaneous member may be deflected 15 to 90 degrees relative to the initial parallel motion, it will be understood that the transcutaneous member guide portion of the fluid delivery device may be constructed to deflect the transcutaneous member to an angle less than 15 degrees or more than 90 degrees relative to the initial parallel motion. In many applications of the sampling or fluid delivery device of the present invention, it is preferred to deliver the fluid from the device to the patient or to withdraw fluid from the patient via a flexible transcutaneous member which is inserted into the skin of the patient. The flexible transcutaneous member is more comfortable when maintained in the skin of the patient than a rigid needle, particularly in the case of an active patient whose movements may cause discomfort or pain with a rigid transcutaneous member in place in the patient's skin. However, because the flexible transcutaneous member cannot be injected into the skin by itself, the flexible transcutaneous member is mated with a rigid transcutaneous member to facilitate the injection of the flexible transcutaneous member into the skin of the patient.

The following fluid delivery devices include both a rigid or semirigid transcutaneous member having a sharpened penetrating member coupled with a flexible transcutaneous member, which may be constructed from medical grade silicone, PVC or other suitable materials. In these embodiments, the rigid transcutaneous member is disposed within the lumen of the flexible transcutaneous member. The rigid transcutaneous member may be hollow, for delivering or withdrawing the fluid therethrough, or it may be solid, wherein the fluid is delivered or withdrawn around the rigid transcutaneous member through the lumen of the flexible transcutaneous member.

In these embodiments, the penetrating member of the rigid transcutaneous member is first driven into the skin of the patient and the flexible transcutaneous member follows the rigid cannula into the skin after the skin has been punctured by the penetrating member. The penetrating member of the rigid transcutaneous member is then retracted into the flexible transcutaneous member so that the flexible transcutaneous member acts as a cushion between the patient and the penetrating member. The penetrating member may be retracted to its original position within the housing, to a position between its original position and its deployed position, or to a position further away from its deployed position than its original position. The position of the rigid transcutaneous member between the original position and the deployed position is preferred because the rigid transcutaneous member helps to prevent any kinking that may occur in the flexible transcutaneous member between the housing and the patient's skin.

In order to insure that then flexible transcutaneous member does not retract along with the transcutaneous member cannula, a retention device may be built into either the flexible transcutaneous member or the exit port to retain the flexible transcutaneous member in its fully deployed position when the rigid transcutaneous member is retracted. An example of an embodiment wherein the flexible transcutaneous member includes a retention device is shown in FIGS. 14A-14C. In these figures, only the relevant portions of the fluid delivery/withdrawal device pertaining to the retention device are shown.

FIG. 14A shows a flexible transcutaneous member 280 and a rigid transcutaneous member 282 disposed within the lumen of the flexible transcutaneous member 280. As shown in FIG. 11A, penetrating member 285 is disposed proximate exit port 286 of first wall 284. As shown, exit port 286 is tapered outwardly of the fluid delivery device. In this embodiment, flexible transcutaneous member 280 includes retention device 288, which, in this embodiment, is in the form of an annular ridge. When the rigid transcutaneous member 282 and the flexible transcutaneous member 280 are driven through the exit port 286, the retention member 288 is also driven through the exit port 286. As can be seen in the figures, retention device 288 causes the flexible transcutaneous member 280 to have a width which is greater than the width of the exit port 286. When the rigid transcutaneous member 282 is retracted in the direction indicated by arrow 290, FIG. 14C, the flexible transcutaneous member 280 is prevented from retracting with the rigid transcutaneous member 282 because the retention device 288 comes into contact with the exit port 286, causing the flexible transcutaneous member to be retained in the deployed position shown in FIG. 14C. As set forth above, the rigid transcutaneous member 282 may be retracted back to its original predeployment position, as shown in FIG. 14C. Alternatively, it may be retracted to a position between the deployed position and the predeployment position or to a position further away from the deployed position than the predeployment position.

Alternatively, the retention device may include one or more barbs located on the flexible transcutaneous member, one or more barbs located directly within the exit port or one or more barbs located on both the flexible transcutaneous member and the exit port.

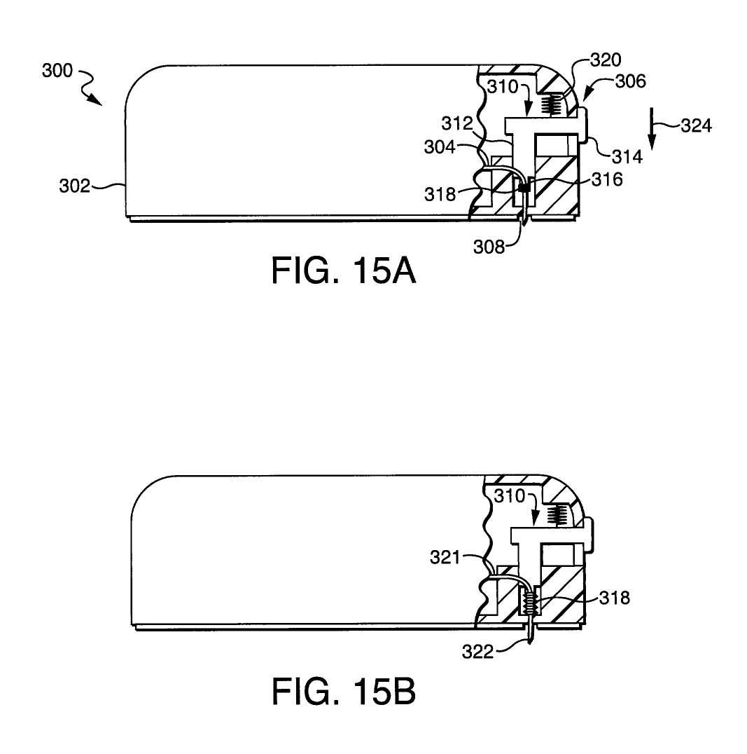

FIGS. 15A and 15B show a further embodiment 300 of the present invention. Device 300 includes a housing 302, transcutaneous member assembly 304, injection actuator 306 and exit port 308. Injection actuator 306 includes a plunger device 310 having a body portion 312, a deployment knob 314 and a transcutaneous member engagement portion 316. A biasing spring 320 is coupled between the body portion 312 and the housing 302. In the predeployment stage shown in FIG. 15A, the biasing spring is in an unenergized state. Although not explicitly shown in FIG. 15A, transcutaneous member assembly 304 includes a rigid transcutaneous member disposed within the lumen of flexible transcutaneous member 321. Flexible transcutaneous member 321 includes a bellows portion 318 which enables the distal end 322 of the flexible transcutaneous member to extend from the housing independent of the rest of the flexible transcutaneous member 321. In the predeployment stage shown in FIG. 12A, the bellows portion is compressed and the distal end 322 of flexible transcutaneous member 321 is within the housing 302.

Deployment of the flexible transcutaneous member into the patient's skin takes place as follows. After the housing is attached to the patient, the patient or other person pushes knob 314 of injection actuator 306 in the direction indicated by arrow 324. This causes the transcutaneous member assembly 304 to be driven into the skin of the patient through exit port 308, as described above with reference to FIGS. 14A-14C. Once the plunger device 310 has reached the end of its travel and both the rigid transcutaneous member and the flexible transcutaneous member 321 have been injected into the skin of the person, biasing spring 320 is extended and energized such that when the knob 314 is released, biasing spring 320 deenergizes, causing the transcutaneous member assembly 304 to be retracted into the housing 302. However, because of the retention device disposed either on the flexible transcutaneous member or within the exit port 308, the distal end 322 of the flexible transcutaneous member 321 is retained in the deployed position shown in FIG. 12B and the bellows portion 318 is fully expanded, which enables the rigid transcutaneous member to be retracted without also retracting the distal end 322 of the flexible transcutaneous member 321. Depending on the particular design of the fluid delivery device, in the deployed position, the rigid transcutaneous member may be retracted to a position that is the same as its predeployment position, to a position that is between the predeployment position and the deployment position, or to a position that is further away from the deployment position than the predeployment position.

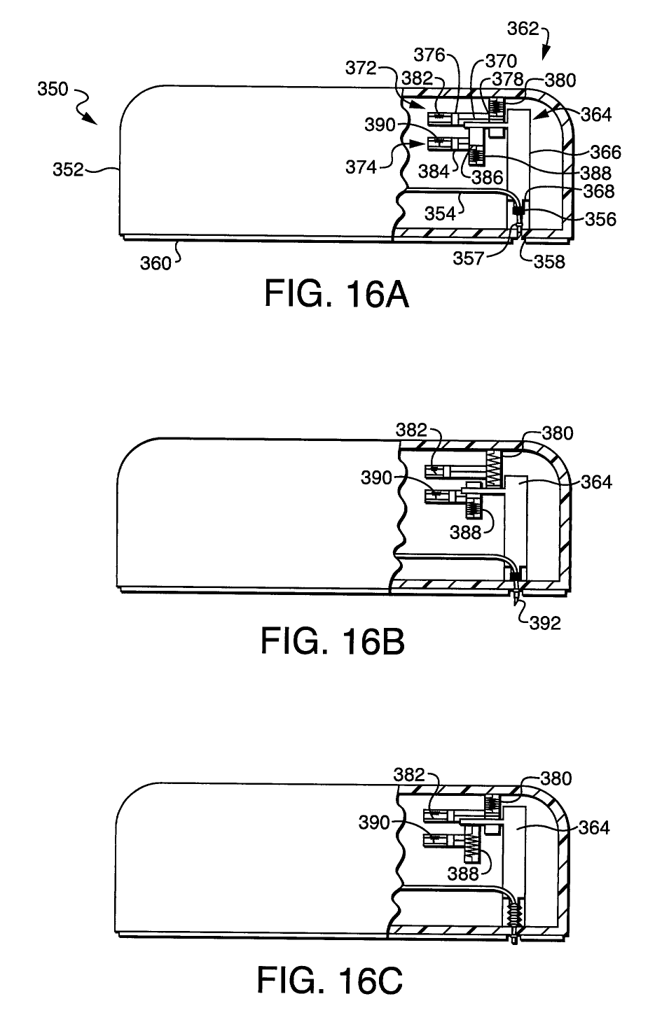

FIGS. 16A-16C show a further embodiment 350 of the present invention. Device 350 includes a housing 352 having an exit port 358 in first wall 360, a transcutaneous member assembly including a flexible transcutaneous member 354 having a bellows portion 356 and retention device 357 and a rigid transcutaneous member (not visible) disposed within the lumen of the flexible transcutaneous member 354 and an injection actuator 362. Injection actuator 362 includes a plunger device 364 including a body portion 366, a transcutaneous member engagement portion 368 and a lateral protrusion 370. Injection actuator 362 further includes deployment latch mechanism 372 and retraction latch mechanism 374. Retraction latch mechanism 372 includes a latch 376 for maintaining a deployment member 378 in a predeployment position against the bias force of deployment spring 380. Deployment latch mechanism 372 further includes an activation device 382, which is preferably in the form of a shape memory alloy or polymer, as described above. Retraction latch mechanism 374 includes a latch 384 for maintaining a retraction member 384 in a predeployment position against the bias force of retraction spring 388. Retraction latch mechanism 374 further includes an activation device 390, which is preferably in the form of a shape memory alloy or polymer.

As shown in FIG. 16B, upon the application of a charge to activation device 382, latch 376 is pulled out of contact with deployment member 378, causing deployment spring 380 to release its energy as it pushes deployment member 378 against lateral protrusion 370, thereby forcing plunger device 364 into the deployment position. In the deployment position, shown in FIG. 16B, both the flexible cannula 354 and the rigid transcutaneous member, including penetrating member 392, are injected into the skin of the person. In this position, retention device 357 is either driven beyond the exit port 358 or is lodged within exit port 258.

Shortly after the transcutaneous member reaches the deployment position shown in FIG. 16B, a charge is applied to activation device 382 of retraction latch mechanism 374 and latch 384 is pulled out of contact with retraction member 384, causing retraction spring 388 to release its energy as it pushes deployment member 378 against lateral protrusion 370, thereby forcing plunger device 364 from the deployment position to the post-deployment position shown in FIG. 16C. Retention device 357 maintains the flexible transcutaneous member 354 in the deployment position, such that, in the post-deployment position, shown in FIG. 16C, the bellows portion 356 of the flexible transcutaneous member 354 is extended and the rigid transcutaneous member is retracted to its predeployment position.

As is shown in FIG. 16C, bellows portion 356, by expanding, enables the rigid transcutaneous member to be retracted while allowing the flexible transcutaneous member to remain in place. Accordingly, in alternative embodiments, bellows portion 356 may be replaced by any type of construction that will enable the rigid penetrator to be retracted without jeopardizing the position of the flexible transcutaneous member in the post-deployment position. One example of such a construction is a sliding joint between the outside diameter of the rigid cannula and the inside diameter of the flexible transcutaneous member. Other constructions will be apparent to those skilled in the art.

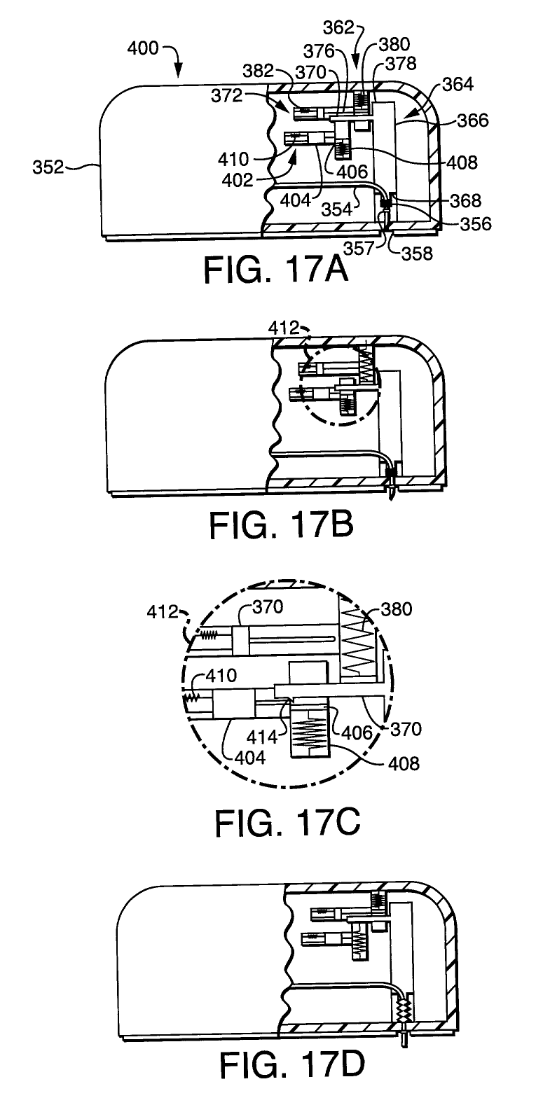

FIGS. 17A-17D show an embodiment 400 which is similar to the device 350 of FIGS. 16A-16C, but in which the retraction latch mechanism is activated automatically and therefore does not require the second activation device. Accordingly, elements of this embodiment which are the same as the fluid delivery device 350 of FIGS. 17A-17C, are referenced with the same reference numerals used in connection with the description of fluid delivery device 350. Fluid delivery device 400 includes a housing 352 having an exit port 358 in first wall 360, a transcutaneous member assembly including a flexible transcutaneous member 354 having a bellows portion 356 and retention device 357 and a rigid transcutancous member (not visible) disposed within the lumen of the flexible transcutaneous member 354 and an injection actuator 362. Injection actuator 362 includes a plunger device 364 including a body portion 366, a transcutaneous member engagement portion 368 and a lateral protrusion 370. Injection actuator 362 further includes deployment latch mechanism 372 and retraction latch mechanism 402. Retraction latch mechanism 372 includes a latch 376 for maintaining a deployment member 378 in a predeployment position against the bias force of deployment spring 380. Deployment latch mechanism 372 further includes an activation device 382, which is preferably in the form of a shape memory alloy or polymer, as described above. Retraction latch mechanism 402 includes a latch 404 for maintaining a retraction member 406 in a predeployment position against the bias force of retraction spring 408. Retraction latch mechanism 402 further includes a latch spring 410, for biasing latch 404 in the position shown in FIG. 17A, wherein latch 404 contacts retraction member 406.

As shown in FIG. 17B, upon the application of a charge to activation device 382, latch 376 is pulled out of contact with deployment member 378, causing deployment spring 380 to release its energy as it pushes deployment member 378 against lateral protrusion 370, thereby forcing plunger device 364 into the deployment position. In the deployment position, shown in FIG. 16B, both the flexible transcutaneous member 354 and the rigid transcutaneous member, including penetrating member 392, are injected into the skin of the person. In this position, retention device 357 is either driven beyond the exit port 358 or is lodged within exit port 258.

FIG. 17C shows detailed portion 412 of FIG. 17B. As shown in FIG. 17C, lateral protrusion 370 of plunger device 364 includes a ramp portion 414 positioned thereon such that, when the plunger device 364 reaches the deployment position shown in FIG. 17B, ramp portion 414 urges latch 404 out of contact with retraction member 406, thereby enabling retraction spring 408 to deenergize and retract the plunger device to the post-deployment position shown in FIG. 17D. Retention device 357 maintains the flexible cannula 354 in the deployment position, such that, in the post-deployment position, shown in FIG. 17D, the bellows portion 356 of the flexible transcutaneous member 354 is extended and the rigid transcutaneous member is retracted to its predeployment position.

Again, alternative constructions of the bellows portion that will enable the rigid penetrator to be retracted without jeopardizing the position of the flexible transcutaneous member in the post-deployment position, such as the sliding joint, may be utilized in these embodiments. Other constructions will be apparent to those skilled in the art.

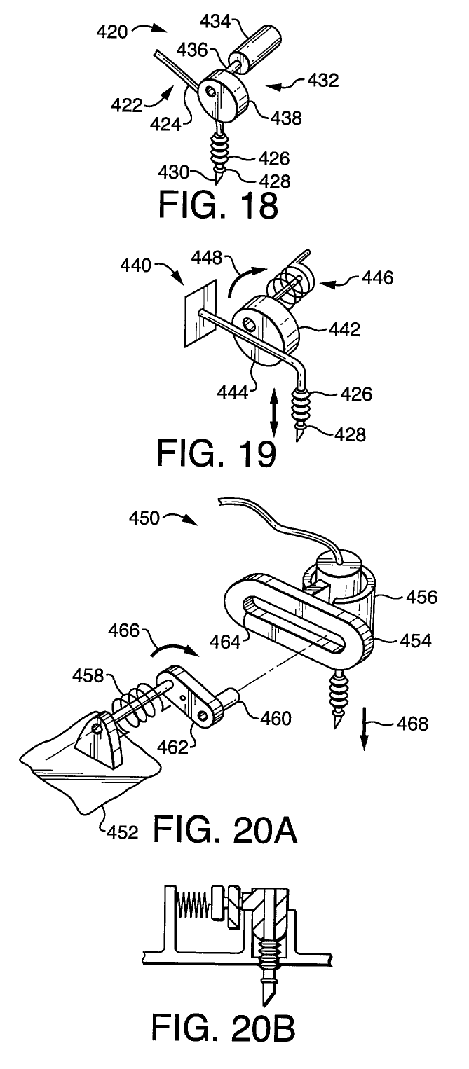

FIG. 18 shows yet another embodiment 420 of the present invention. In connection with this embodiment, and the several embodiments that follow, only the injection actuator and transcutaneous member assembly are shown and described. It will be understood that the injection actuator and transcutaneous member assembly described in connection with these embodiments will be housed in a housing similar to those previously described. Transcutaneous member assembly 422 includes a flexible transcutaneous member 424 having a bellows portion 426 and a retention device 428. A rigid transcutaneous member having a penetrating member 430 is disposed within the lumen of the flexible transcutaneous member 424. Injection actuator 432 includes a driving mechanism 434 for driving axle 436 which is coupled to urging device 438. Driving mechanism 434 may comprise a motor, spring or any device that is capable of causing axle 436 to rotate at least one revolution. In this embodiment, urging device 438 is in the form of a disk and axle 436 is coupled thereto at a point offset from the center of the disk. When the driving mechanism 434 is activated and causes the axle 436 to rotate, the portion of urging device 438 opposite the axle 436 pushes the transcutaneous member assembly 422 to the deployment position described above. In the preferred embodiment, the transcutaneous member assembly 422 is biased in the predeployment position shown in FIG. 18 such that, after the urging device pushes the transcutaneous member assembly 422 into the deployment position and continues to rotate, the transcutaneous member assembly returns to the predeployment position under the force of the biasing means coupled to the assembly. As described above, the bellows portion 426 and retention device 428 enable the flexible transcutaneous member 422 to remain in the deployed position while the rigid transcutaneous member and penetrating member 430 are retracted.

FIG. 19 shows an embodiment 440 which is similar to the device 420 of FIG. 15. However, urging member 442 includes a retention device 444 for retaining the transcutaneous member assembly in contact with the urging device 442. Rather than rotating the axle a complete revolution, driving mechanism 446, which may be a prewound spring, as shown, a bi-directional motor, or other driving means, rotates the urging member one quarter turn in the direction indicated by arrow 448, to drive the transcutaneous member assembly to the deployment position, and one quarter turn in the direction opposite that indicated by arrow 448, to retract the transcutaneous member assembly to the post-deployment position. As described above, the bellows portion 426 and retention device 428 enable the flexible transcutaneous member 422 to remain in the deployed position while the rigid transcutaneous member and penetrating member 430 are retracted.

FIGS. 20A and 20B show an embodiment 450 which includes a driving mechanism 452 which is coupled to a force translator 454 which in turn is coupled to transcutaneous member assembly 456. In the preferred embodiment, driving mechanism 452 includes a torsion spring which is energized before protrusion 460 of lever arm 462 is inserted into slot 464 of force translator 454. FIG. 20B is a side view of the embodiment 450 in such a configuration. When the torsion spring 458 is released, it lever arm 462 and protrusion 460 to rotate in the direction indicated by arrow 466, causing protrusion 460 to drive the force translator 454 and transcutaneous member assembly 456 in the direction indicated by arrow 468 during the first 45 degrees of rotation, thereby injecting the rigid transcutaneous member and flexible transcutaneous member into the skin of the person, and then to drive the force translator 454 and transcutaneous member assembly 456 in the direction opposite that indicated by arrow 468 during the second 45 degrees of rotation, thereby retracting the rigid transcutaneous member. The flexible transcutaneous member maintains its deployment position with the aid of the bellows portion and the retention device.

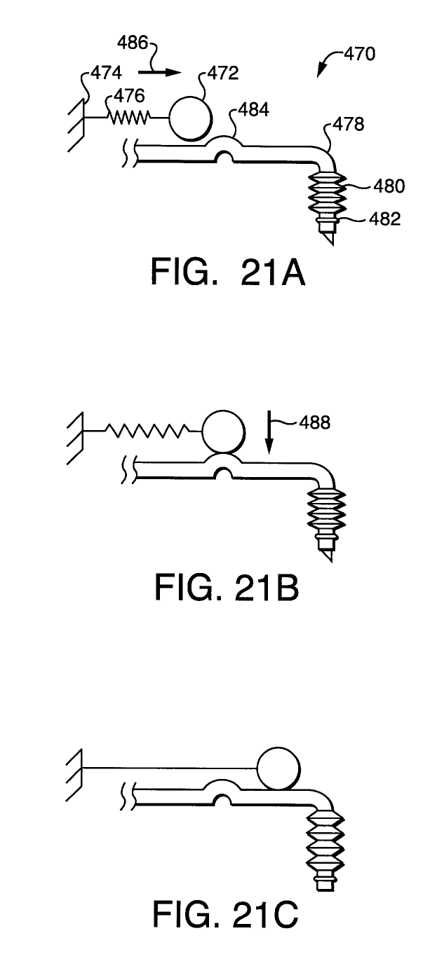

FIG. 21C-21C shows another embodiment 470 of the invention including an urging device 472 which is coupled to a portion 474 of the housing of the associated fluid delivery device by a spring 476. Transcutaneous member assembly 478 includes a flexible transcutaneous member having a bellows portion 480 and preferably a retention device 482. A rigid transcutaneous member is disposed within the lumen of the flexible transcutaneous member. transcutaneous member assembly 478 includes a protrusion 484, which may comprise a bend in the rigid and flexible transcutaneous members, as shown in the figure, or a ramp portion mounted on the transcutaneous member assembly. In the predeployment position shown in FIG. 21A, the spring 476 is maintained in an energized state by a latch assembly (not shown) such that the urging device 472 is positioned one side of the protrusion 472. Upon deenergization of the spring 476, the urging device 472 is driven in the direction indicated by arrow 486. Urging member 472 is constructed and mounted within the housing such that it is maintained in its plane of travel as the spring 476 is deenergized. Upon contacting protrusion 484, urging device 472 exerts a force thereon, causing transcutaneous member assembly 478 to be driven in the direction indicated by arrow 488 from the predeployment position to the deployed position. As the urging member 472 passes over the protrusion 484, the transcutaneous member assembly, which is biased in the predeployment position, travels in the direction opposite that indicated by arrow 488 from the deployed position to the predeployment position, as shown in FIG. 21C. The flexible transcutaneous member maintains its deployment position with the aid of the bellows portion and the retention device.

In further embodiments of the invention, in order to enable the flexible transcutaneous member to remain in the deployed position while retracting the rigid transcutaneous member, the end of the flexible transcutaneous member opposite the end that is injected into the person is constructed of a sealing portion which forms a fluid seal with the rigid transcutaneous member that allows the flexible transcutaneous member to move within the flexible cannula while maintaining the fluid integrity of the fluid delivery device and while enabling the retention device to hold the flexible transcutaneous member in the deployed position.

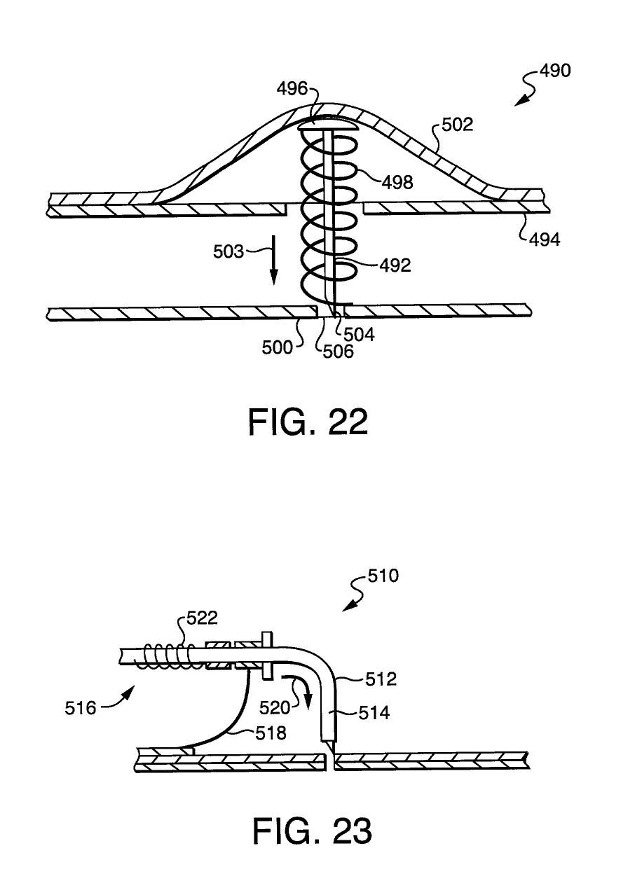

FIGS. 22 and 23 show two embodiments that utilize this type of transcutaneous member assembly. Embodiment 490 of FIG. 22 includes a transcutaneous member assembly 492 having a rigid transcutaneous member within a transcutaneous member cannula. Both are mounted within a housing 494 of a fluid delivery/withdrawal device. The rigid transcutaneous member includes a head portion 496 which extends from the housing 494. A return spring is mounted between the head portion 496 of the rigid transcutaneous member and the wall 500 of housing 494 to bias the transcutaneous member assembly in the position shown in the figure, which is the predeployment position. An optional membrane 502 may be mounted over the transcutaneous member assembly to protect the integrity of the housing 494. In operation, the head portion of the transcutaneous member assembly is pushed in the direction indicated by arrow 503 to cause the flexible transcutaneous member and the penetrating member 504 of the rigid transcutaneous member to be driven out of exit port 506 and into the skin of the person. When the head portion 496 is released, spring 492 is deenergized, causing the rigid transcutaneous member to be driven in the direction opposite that indicated by arrow 503. However, the flexible transcutaneous member, with the aid of a retention device mounted thereon or on the exit port, is held in place in the deployed position while the rigid transcutaneous member is retracted.

FIG. 23 shows an embodiment 512 having a transcutaneous member assembly 514 disposed within a transcutaneous member guide 512. Injection actuator 516 includes a deployment spring 518 for driving the transcutaneous member assembly 514 through guide 512 in the direction indicated by arrow 520 and a retraction spring 522, which is coupled between the housing (not shown) and the rigid transcutaneous member. When deployment spring 518 reaches the end of its travel, it loses contact with the transcutaneous member assembly 514 and retraction spring 522, which is now energized, deenergizes, causing the rigid transcutaneous member to be pulled in the direction opposite that indicated by arrow 520. A retention device associated with the fluid delivery device maintains the flexible transcutaneous member in the deployed position while the rigid transcutaneous member is retracted.

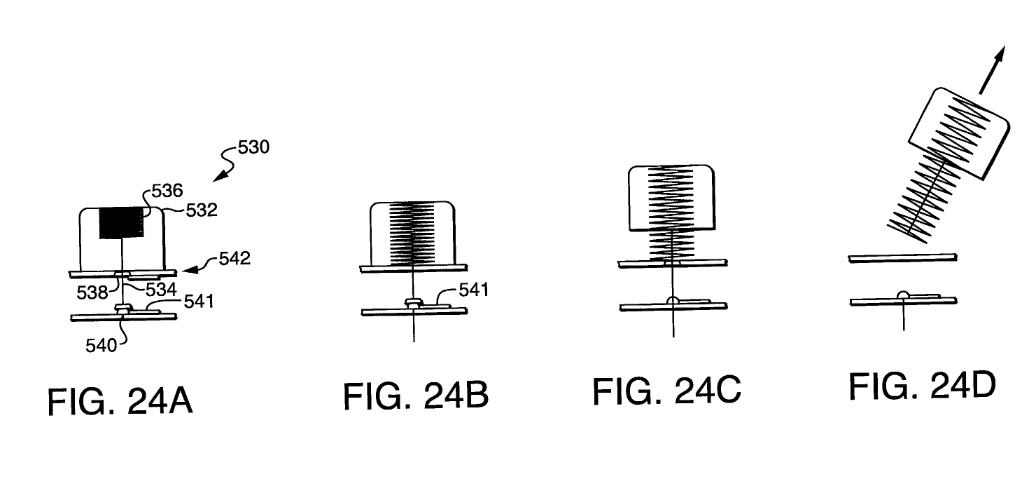

FIGS. 24A-24D show an embodiment 530 including a secondary housing 532 including a transcutaneous member assembly 534 and a deployment spring 536. In the predeployment position, spring 536 is compressed and energized, and held in this state by a latch mechanism (not shown). The flexible transcutaneous member 541 of the transcutaneous member assembly is housed within the housing 542 and the rigid transcutaneous member is inserted into the housing 542 and into flexible transcutaneous member 541 through a port 538 such that the penetrating member of the rigid transcutaneous member and the distal end of the flexible transcutaneous member are proximate exit port 540. Upon releasing the latch mechanism, deployment spring 536 deenergizes and drives the transcutaneous member assembly, including the flexible cannula 541, through the exit port 540 and into the skin of the person. This deployment position is shown in FIG. 24B. The secondary housing can then be removed from the housing 542 and discarded, FIGS. 24C and 24D, or reloaded for the next use.

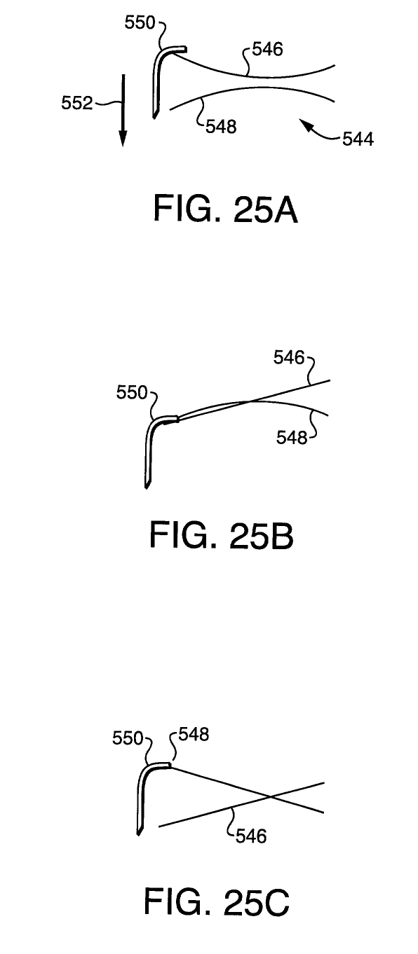

FIGS. 25A-25C shown yet another embodiment 544 of the injection actuator. This embodiment 544 includes a deployment spring 546 coupled between the transcutaneous member assembly 550 and the housing (not shown) and a retraction spring 548 in a preloaded state, FIG. 25A. When the deployment spring 546 is released, it drives the transcutaneous member assembly in the direction indicated by arrow 552 into the skin of the person. At the end of the travel of the deployment spring 546, transcutaneous member assembly 550 comes into contact with retraction spring 548 while deployment spring 546 loses contact with the transcutaneous member assembly 550, FIG. 25B. Retraction spring 548 is then activated, thereby driving transcutaneous member assembly 550 in the direction opposite that indicated by arrow 552 to retract the rigid transcutaneous member, FIG. 25C, while the flexible transcutaneous member remains in the deployed position.

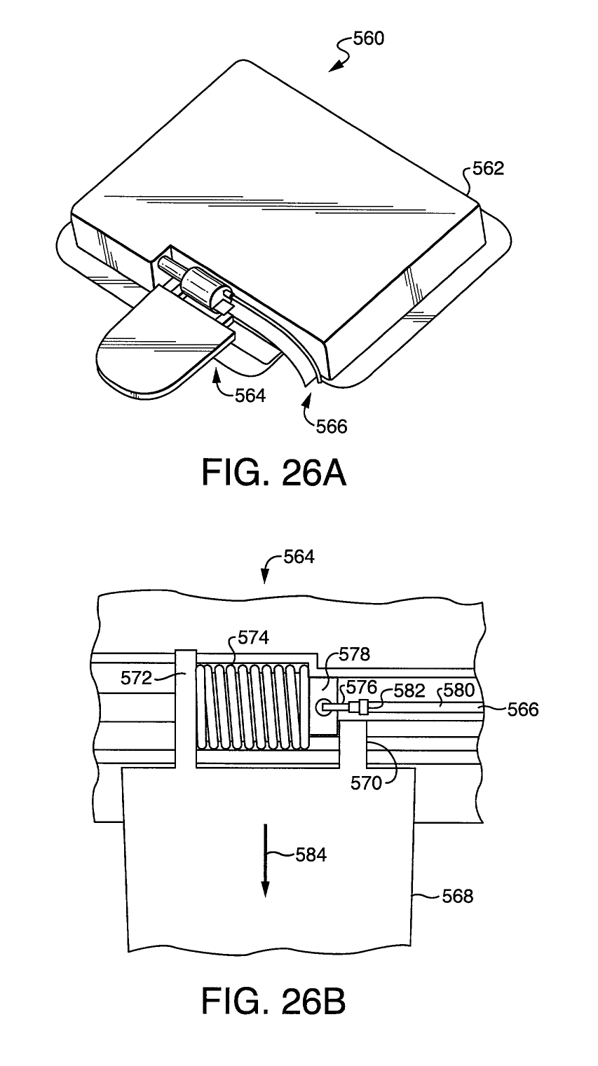

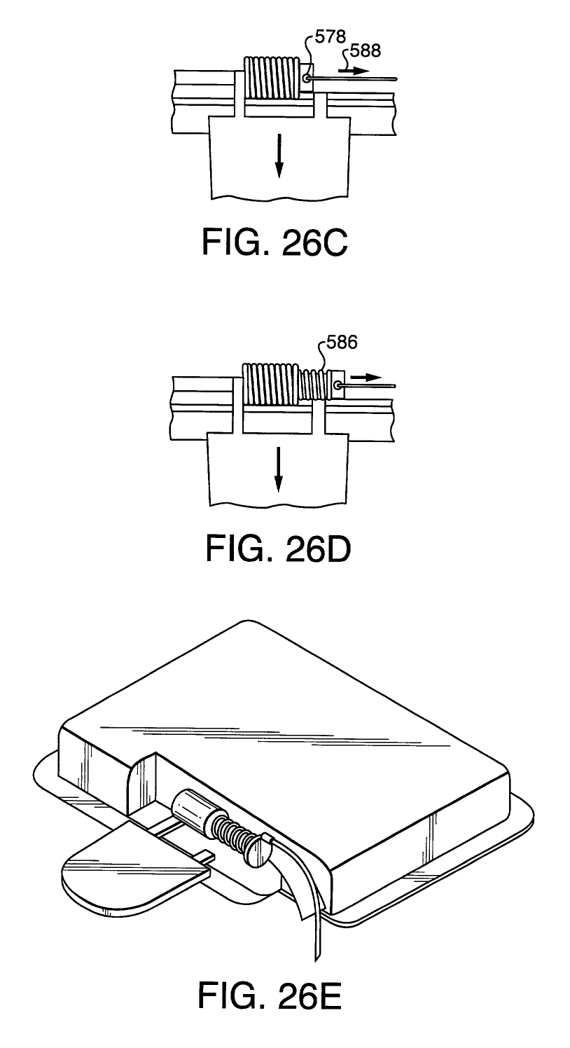

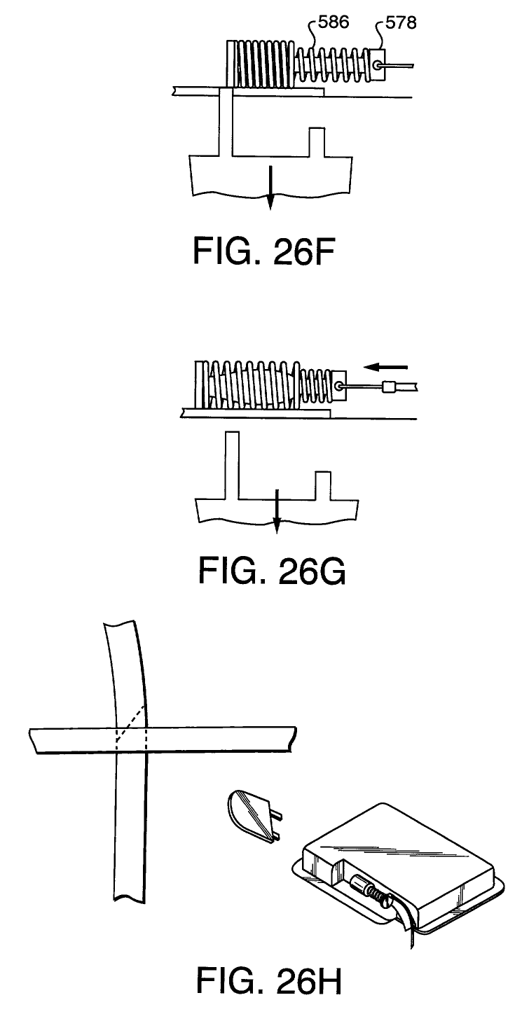

FIGS. 26A-26H show another embodiment 560 of the present invention. Device 560 includes a housing 562, an injection actuator 564 and a transcutaneous member assembly 566, FIG. 26A. As shown in FIG. 26B, injection actuator 564 includes an activation tab 568 having a deployment protrusion 570 and a retraction protrusion 572. A deployment spring, which is not visible in FIG. 26B, is disposed within a retraction spring 574 such that a longitudinal axis of the deployment spring coincides with a longitudinal axis of the retraction spring 574. Transcutaneous member assembly 566 includes a rigid transcutaneous member 576 coupled at a proximate end thereof to a head portion 578. A flexible transcutaneous member 580 is disposed on the rigid transcutaneous member 576 and includes a sliding seal portion which, as described above, enables the rigid transcutaneous member 576 to move relative to the flexible transcutaneous member while maintaining a fluid seal therebetween. The deployment spring and retraction spring 574 are coupled together at their ends proximate the retraction protrusion 572. The other, distal end of retraction spring 574 is prevented from moving toward the transcutaneous member assembly by a retaining member (not shown). Alternatively, in place of the sliding seal portion, flexible transcutaneous member 580 may include a bellows portion, as described above, for enabling the rigid transcutaneous member 576 to be retracted independent of the flexible transcutaneous member 580. Other embodiments that will enable independent movement between the rigid and flexible transcutaneous members will be apparent to those skilled in the art.

The operation of device 560 begins when tab 568 is pulled in the direction indicated by arrow 584. Since deployment protrusion 570 is shorter than retraction protrusion 572, deployment spring 586, FIG. 26D, which was held in an energized state by the deployment protrusion 570, is allowed to deenergize and drive the head portion 578 of transcutaneous member assembly 566 in the direction indicated by arrow 588. This causes the head portion 578 to drive the rigid and flexible transcutaneous members through the exit port of the housing 562 and into the skin of the person.

The difference in length between the deployment protrusion 570 and the retraction protrusion 572 is such that the deployment spring 586 is allowed to substantially fully deenergize before the retraction spring 574 is released by retraction protrusion 572. When retraction spring 574 is released by the retraction protrusion 572, FIGS. 26F-26G, retraction spring 574 deenergizes by exerting a force on the end of deployment spring 586 to which it is coupled. The presence of the retaining member causes the retraction spring to drive the head portion 578 and rigid transcutaneous member 576 in the direction opposite that indicated by arrow 588. As shown in FIG. 26H, after both the deployment spring 586 and retraction spring 574 have both been deenergized as described above, the flexible transcutaneous member 580 is injected into the skin of the person and the rigid transcutaneous member 576 and its penetrating member are retracted within the flexible transcutaneous member 580 to a position which may be anywhere between the deployed position of the flexible transcutaneous member 580 and the predeployed position shown in FIG. 26B. Alternatively, the rigid transcutaneous member 576 may be retracted to a position which is further away from the deployed position than the predeployment position. Flexible transcutaneous member 580 is held in the deployment position by the retention device, which may be one or more barbs disposed on either or both of the flexible transcutaneous member 580 and the exit port, as described below.

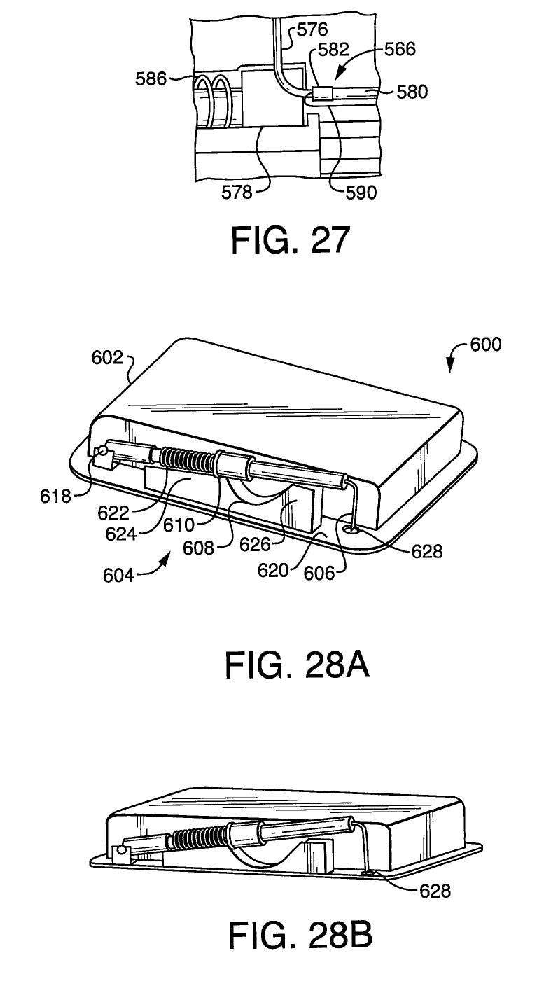

Alternatively, the retention device may include an interference member with which the sealing portion 582 of the flexible transcutaneous member comes into contact when the flexible transcutaneous member reaches the deployed position, wherein the interference member maintains the flexible transcutaneous member 580 in the deployed position when the rigid transcutaneous member 576 is retracted. Such a configuration is shown in FIG. 27, which depicts the deployment spring 586, head portion 578 and flexible transcutaneous member 580. As the transcutaneous member assembly 566 reaches the deployed position, interference member 590 contacts the sealing portion 582 of flexible transcutaneous member, thereby retaining the flexible transcutaneous member 580 in the deployed position while the rigid transcutaneous member 576 and head portion 578 are retracted.

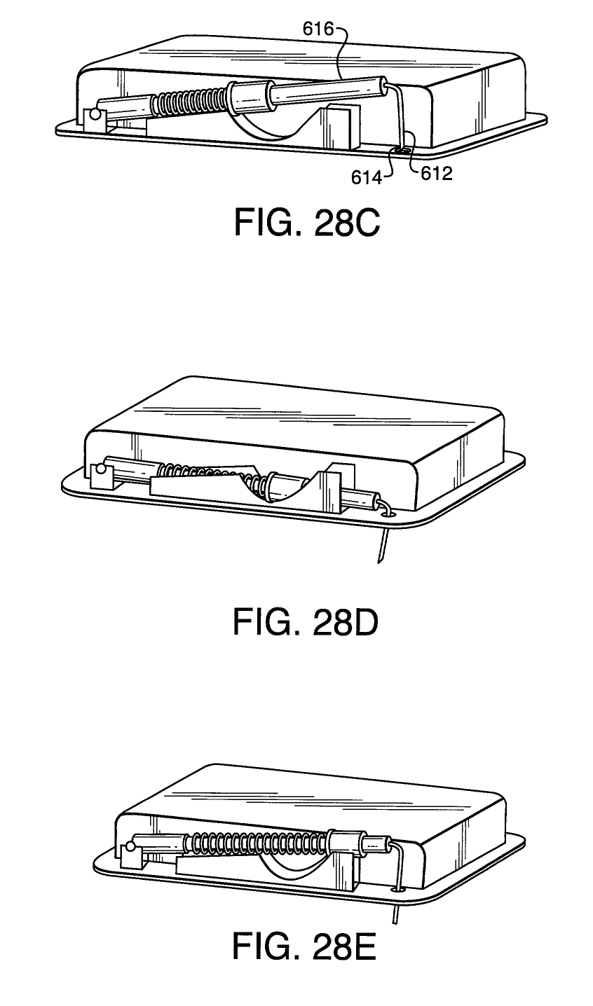

FIGS. 28A-28E show another embodiment 600 of the present invention. Device 600 includes a housing 602, an injection actuator 604 and a transcutaneous member assembly 606. Injection actuator 604 includes a cam follower assembly having a cam portion 608 and follower portion 610. Transcutaneous member assembly 606 includes a rigid transcutaneous member 614 disposed within a flexible transcutaneous member 612, both of which being disposed within a sleeve 616 along which cam follower portion 610 travels. Sleeve 616 is mounted to housing 602 at a pivot 618 and is biased toward the first wall 620. Injection actuator 604 further includes a spring 622 which is mounted between pivot 618 and cam follower 610. In the predeployment position shown in FIG. 28A and 28B, cam follower 610 is disposed on first ramp portion 624 of injection actuator device 604 and maintained in the position shown relative to the pivot 618 by a latch mechanism (not shown). In this position, spring 622 is in a compressed, energized state. Upon releasing the latch mechanism, spring 622 deenergizes and drives cam follower 610 along first ramp portion 624 and into cam portion 608, FIG. 28C. As cam follower portion slides into the cam, the transcutaneous member assembly 606 is driven toward first wall 620, out of the housing 602 through exit port 628 and into the skin of the person, FIG. 28D. As cam follower portion 610 continues to be driven by spring 622, it follows cam portion 608 up onto second ramp portion 626, which causes transcutaneous member assembly 606 to be lifted away from first wall 620, thereby retracting rigid cannula 604. Flexible transcutaneous member 612 is maintained in the deployed position shown in FIG. 28E, while rigid transcutaneous member 604 is retracted by the interference fit between the exit port 628 and a retraction prevention device (not shown), such as is described above. A bellows portion or sliding joint, both described above, may be utilized in connection with the flexible transcutaneous member to allow the rigid transcutaneous member to be retracted independently of the flexible transcutaneous member.

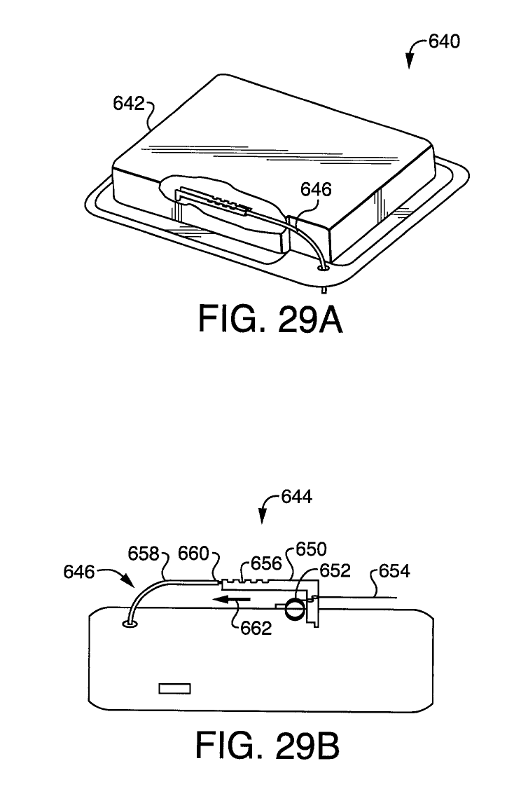

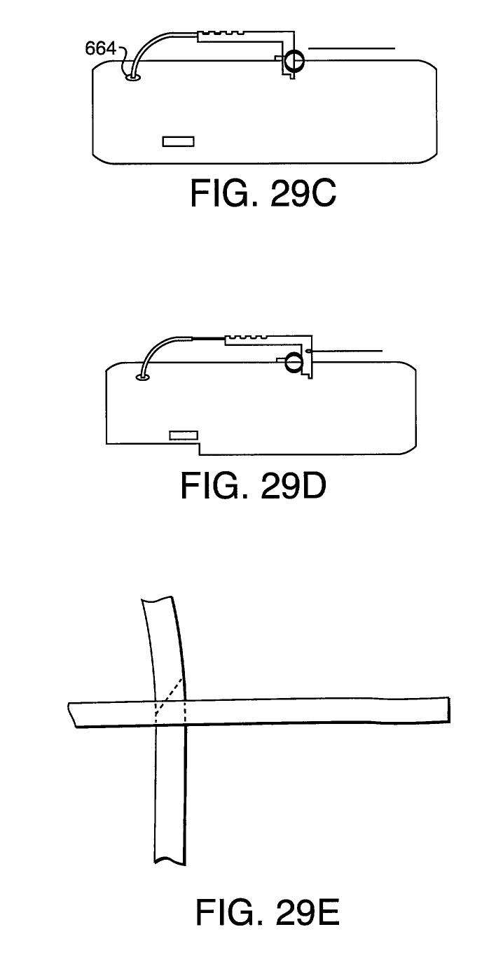

FIGS. 29A-29E show yet another embodiment 640 of the present invention. Device 640 includes a housing 642, an injection actuator 604 and a transcutaneous member assembly 646, FIG. 29A. Injection actuator 644 includes a deployment yoke 650, a spring 652 and a latch mechanism 654, FIG. 29B. Spring 652 is preferably a torsion spring having one end thereof mounted to the housing 642 and the other end mounted to the deployment yoke 650. In the predeployment position shown in FIG. 29B, torsion spring 652 is maintained in an energized state by a latch mechanism 654.

Transcutaneous member assembly 646 includes a rigid transcutaneous member 656 having a proximal end thereof coupled to the deployment yoke 650 and a flexible transcutaneous member 658 having a sealing portion 660 through which the rigid transcutaneous member 656 extends. Latch assembly 654 may be a mechanical latch or an electrically-activated latch formed, for example, from a shape memory alloy or polymer which contracts upon the application of an electrical charge thereto.

Upon activation of the latch mechanism 654, spring 652 is released and begins to deenergize. As it deenergizes, it drives deployment yoke 650, along with transcutaneous member assembly 646 in the direction indicated by arrow 662. This causes the transcutaneous member assembly to be driven out from the housing 642 through exit port 664 and into the skin of the person, FIG. 26C. As the spring 652 continues to deenergize by rotating its end that is coupled to the yoke 650, after the rigid transcutaneous member 656 and flexible transcutaneous member 658 have been injected into the person, the spring 652 drives the yoke away from the exit port in the direction opposite that indicated by arrow 662, thereby retracting the rigid cannula 652, FIG. 29D. The flexible transcutaneous member 658 remains in the deployed position shown in FIGS. 29D and 29E with the aid of a retention device such as described above.

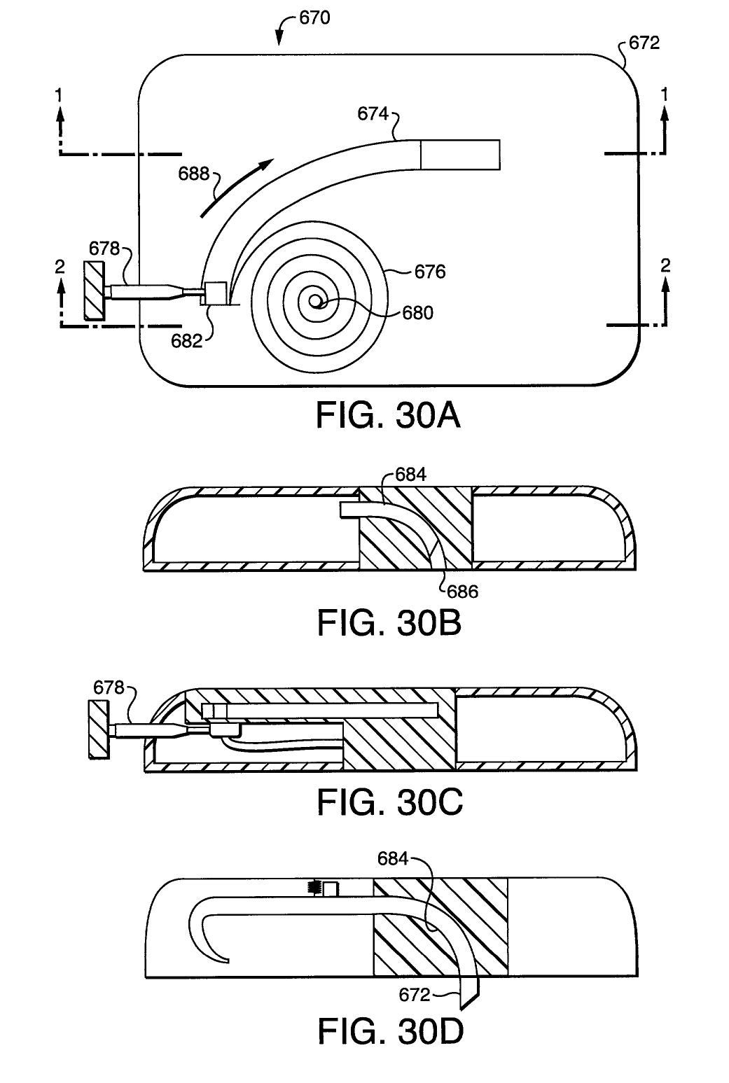

FIGS. 30A-30D show another embodiment 670 of the present invention. Device 670 includes a housing 672, a transcutaneous member assembly 674, a spring 676 and a latch mechanism 678. FIG. 30B is a cross-sectional view along line 1-1 of FIG. 30A, which shows that housing 672 includes a transcutaneous member guide portion 684 which guides the transcutaneous member assembly 674 out of the housing 672 via exit port 686. Spring 676 is preferably a torsion spring having one end 680 coupled to the housing and the other end 682 coupled to the transcutaneous member assembly 674. In the predeployment state shown in FIG. 30A, spring 676 is energized and transcutaneous member assembly 674 is maintained in its predeployment position by latch mechanism 678. Upon releasing latch mechanism 678 by pulling it from the housing 672, spring 676 is allowed to deenergize and drive transcutaneous member assembly 674 in the direction indicated by arrow 688 such that, with the aid of transcutaneous member guide portion 684, transcutaneous member assembly 674 is driven through exit port 686 and into the skin of the person. As shown in FIG. 30C, which is a crosssection view along line 2-2 of FIG. 30A, spring 676 is able to be mounted in a plane parallel to the skin of the person, which enables the size of the housing 672 to be reduced. Generally, the transcutaneous member assembly 674 is constructed to enable it to follow the arc of travel of end 682 of spring 676 as it deenergizes. FIG. 30D shows the transcutaneous member assembly 674 injected into the skin of the person through exit port 686 and transcutaneous member guide portion 684.

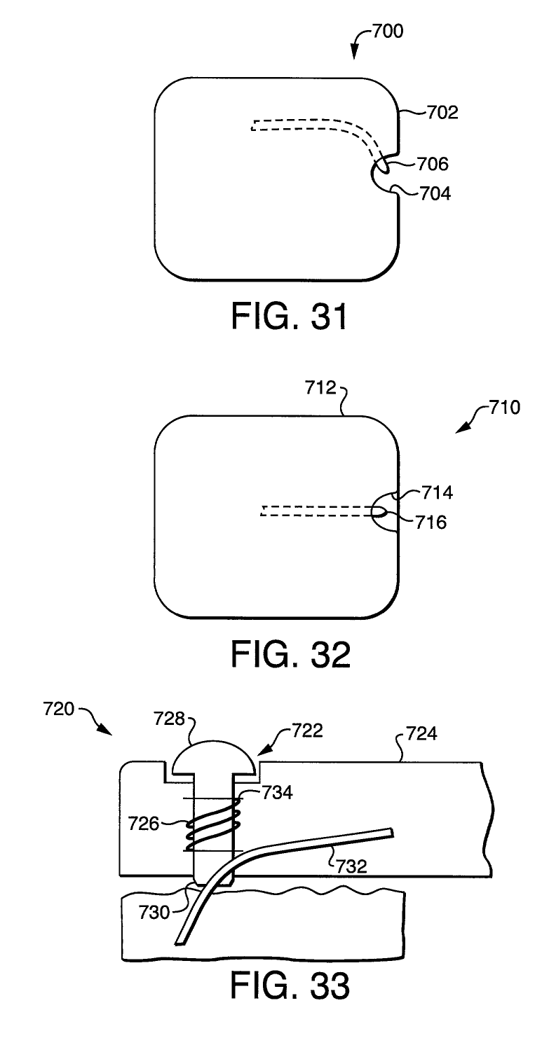

In the devices of the present invention, it may be desirable to be able to view the site where the rigid transcutaneous member or the rigid and flexible transcutaneous member have entered the skin of the person in order to inspect the site for infection or other concerns. Accordingly the housing of a device of the present invention may be modified to provide a viewing area. FIG. 31 shows an embodiment 700 which includes a housing 702 having a contour portion 704 and a transcutaneous member assembly 706. Contour portion 704 enables the transcutaneous member assembly 706 to be driven out of a side wall of the housing and into the skin of the person, while providing protection for the injection site on three sides thereof. FIG. 32 shows an embodiment 710 which includes a housing 712 having a window portion 714 and a transcutaneous member assembly 716. Window portion 714 preferably is formed from a transparent material such as plastic, fits flush with the shape of the housing 712 and enables the person to view the injection site of the transcutaneous member assembly 716.

It will be understood that most or all of the embodiments of the device of the present invention which have been described herein may be used in connection with the housings 702 and 712 to provide a viewing area of the injection site.

FIG. 33 shows another embodiment 720 including a plunger device 722 mounted within a housing 724. This embodiment operates similar to the embodiment described with reference to FIGS. 6A-6C, wherein plunger device 722 includes a body portion 726, a head portion 728 and a transcutaneous member engagement portion 730 for engaging transcutaneous member 732. In the embodiment, however, plunger device 722 is formed from a transparent material which enables the injection site to be seen therethrough. A spring 734 biases the plunger device 722 against the injection site to provide a clear view of the site through the plunger device 722. In one embodiment, plunger device 722 is constructed in such a way that the view of the injection site is magnified when viewed through the head portion 758 of the plunger device 722. In another embodiment, a light source (not shown) may be directed at the plunger device 722 to illuminate the injection site.

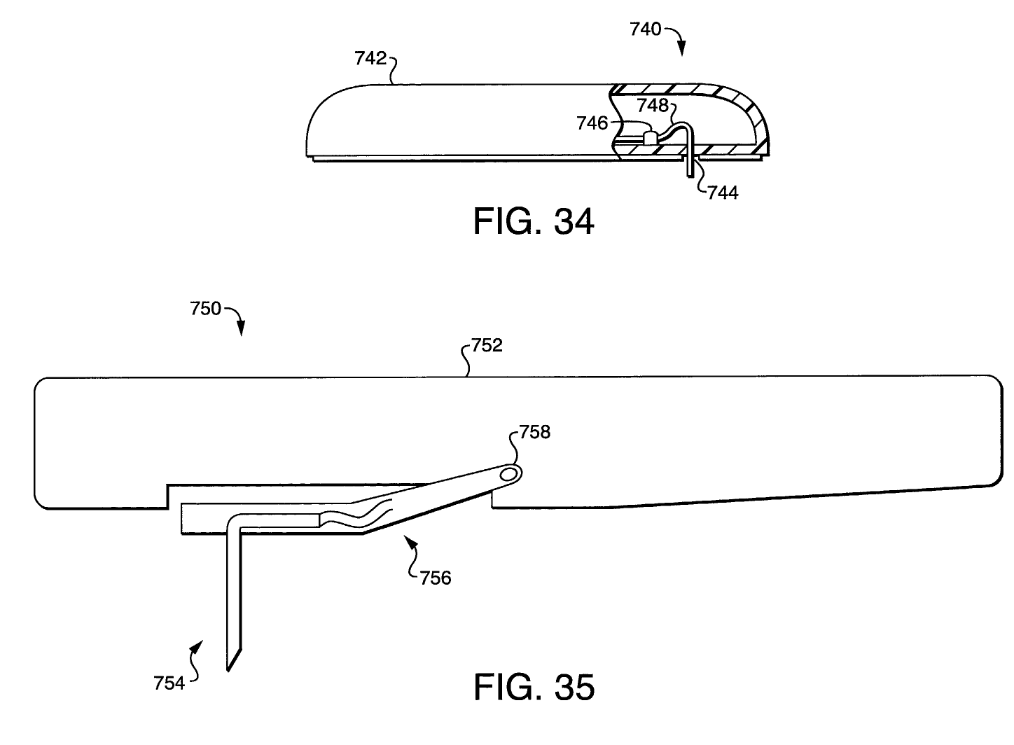

One advantage of the fluid delivery device of the present invention is that it requires only one small housing to be attached to the person. In contrast to prior art fluid delivery devices, which may have included multiple bulky parts, the present invention enables the person to be more active while wearing the fluid delivery device than would be the case with the prior art devices. However, it is important to maintain the transcutaneous member assembly in the proper deployed position throughout the period that the device is attached to the person, despite the movement and activity of the person. Since the fluid delivery devices of the present invention are typically attached to the abdominal area of the person, normal body motion and bending could cause a portion of the housing to flex away from the skin. Over time, a transcutaneous member which is rigidly fixed with respect to the housing may have the tendency to creep out of the injection site, which may result in the transcutaneous member completely pulling out of the injection site, or in a flexible transcutaneous member developing enough slack to cause kinking in the transcutaneous member. FIGS. 34-37 show embodiments of the present invention which enable the housing of the fluid delivery device to move independently of the transcutaneous member assembly, without affecting the position of the transcutaneous member within the person.

FIG. 34 shows an embodiment 740 of the present invention that includes a housing 742 and a transcutaneous member assembly 744. Transcutaneous member assembly 744 preferably includes a flexible transcutaneous member which is attached to the first wall of the housing 742 with a tie-down device 746. The transcutaneous member assembly is injected into the person in such a way that a loop 748 is present between the injection site and the tie-down 746. This loop provides the slack necessary to prevent any tugging on the portion of the transcutaneous member assembly injected into the person if the housing was to be moved away from the injection site.

FIG. 35 shows an embodiment 750 including a housing 752 and a transcutaneous member assembly 754 attached to a strut assembly 756 which is pivotally attached to the housing 752 at point 758. Strut assembly 756 is biased toward the skin of the person, such that, upon any movement of the housing away from the skin, the strut assembly 756 maintains the transcutaneous member assembly in the deployed position shown in the figure.

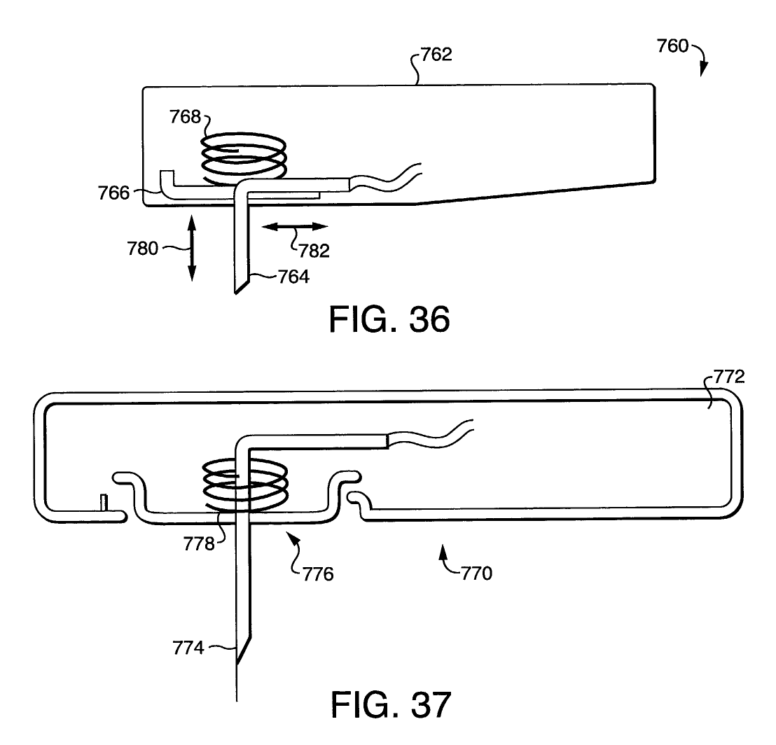

FIG. 36 shows an embodiment 760 including a housing 762 and a transcutaneous member assembly 764 which is coupled to a floating member 766 which is biased against the skin of the person by spring 768. As the person moves, the transcutaneous member assembly 764 and floating member 766 are maintained in contact with the skin, thus enabling the housing to move independently of the transcutaneous member assembly 764 in three dimensions, as shown by arrows 780 and 782.

FIG. 37 shows an embodiment 770 including a housing 772 and a transcutaneous member assembly 774 which is coupled to a floating member 766 which is biased against the skin of the person by spring 768. In this embodiment, the spring 778 is coupled between the transcutaneous member assembly 774 and the floating member 776 to enable the housing 772 to move independently of the transcutaneous member assembly in three dimensions.

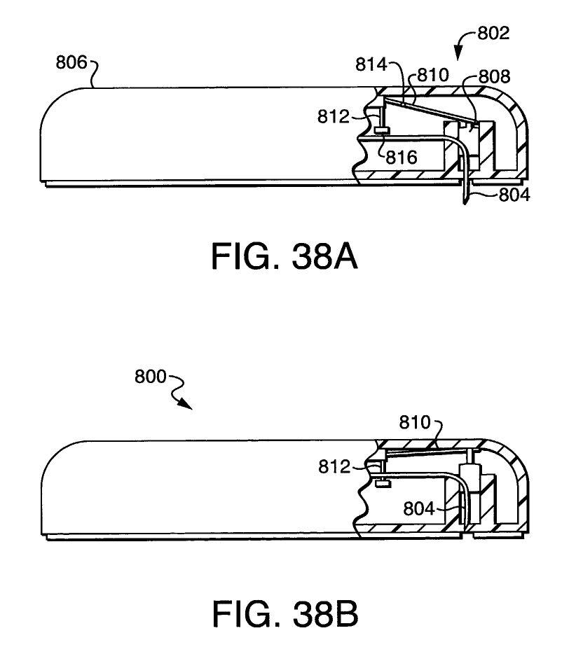

FIGS. 38A-B show an embodiment 800 which includes a housing 806 and a retraction mechanism 802 for retracting a transcutaneous member 804 when the fluid delivery device has completed the infusion and is ready to be removed from the skin of the patient. As shown in FIG. 38A, transcutaneous member 804 is injected into the skin of the person through an exit port of the device 800. Retraction mechanism 802 includes a retraction member 808 coupled to the transcutaneous member 804, a lever 810 coupled at one end to the retraction member 804 and at the other end to an actuator 812. Lever 810 is also coupled to a pivot point 814 of the housing 806. Actuator 812 preferably includes a shape memory alloy or polymer which contracts under the influence of an electrical charge coupled between the lever 810 and a portion 816 of housing 806. However, other devices may be utilized for the actuator 812, such as a piezo electric actuator and a solenoid.

Upon the application of an electrical charge to the actuator 812, by the local processor triggered by a command from the remote control or other means described above, actuator contracts, causing lever 810 to pull retraction member 808 and consequently, transcutaneous member 804 away from the skin of the person, thus retracting the transcutaneous member 804 from the skin of the person, as shown in FIG. 38B. This retraction mechanism 802 may be combined with any of the fluid delivery devices described above having only injection mechanisms, to enable the device to both inject and retract the cannulas.

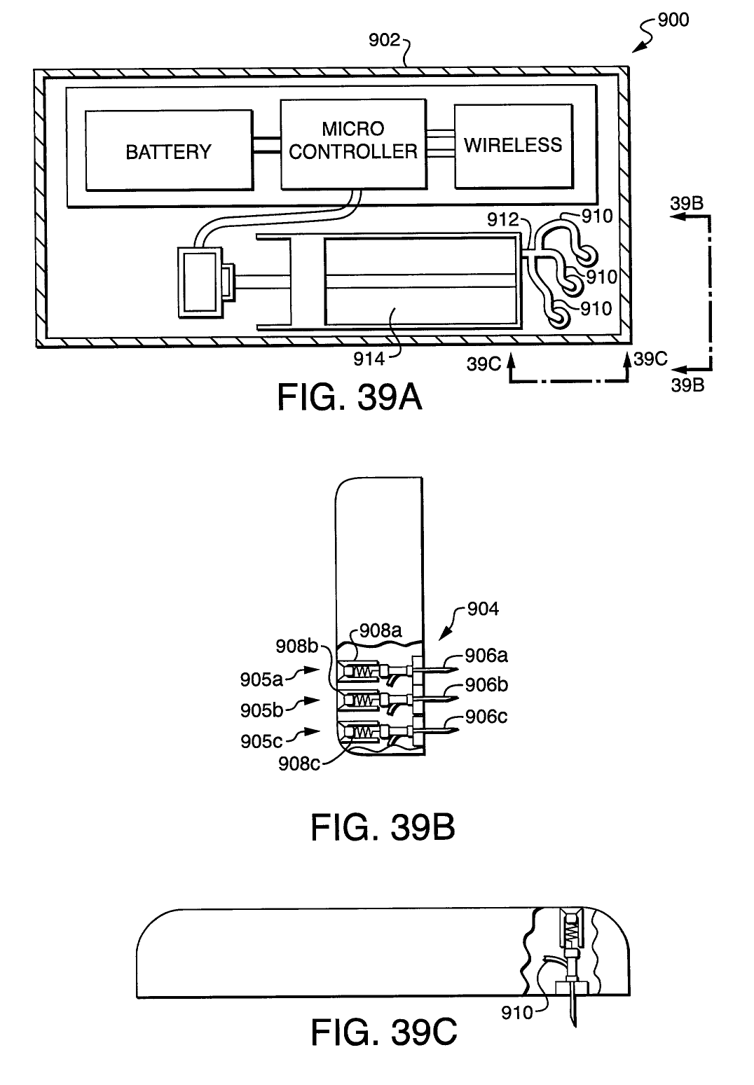

FIGS. 39A-39C show yet another embodiment 900 of the present invention. Device 900 includes a housing 902 for enclosing the electronics, control mechanism and fluid reservoir, as described above. Device 900 further includes a transcutaneous member assembly 904. As shown in FIG. 39A, which is a top view of the device 900, FIG. 39B, which is a side cutaway view of the device 900 as seen from line 39B-39B of FIG. 39A and FIG. 39C, which is a side cutaway view of the device 900 as seen from line 39C-39C of FIG. 39A, transcutaneous member assembly 904 includes three transcutaneous member devices, 905a, 905b and 905c, including transcutaneous member 906a, 906b and 906c and injection actuators 908a, 908b and 908c, respectively. Injection and/or retraction actuators 908a-908c may be constructed according to any of the embodiments described above. Each transcutaneous member device 905 includes a fluid path 910 that branches from a main fluid path 912 which delivers fluid from the reservoir 914 to each cannula 906. The injection actuators are activated individually for a predetermined period of time before the next injection actuator is activated.

Although not specifically shown in the figures, embodiment 900 may be used in connection with a sensor-equipped transcutaneous member as described above to enable extended sensing and fluid delivery functions. The sensor device may also include multiple transcutaneous members for the purpose of carrying out a physiological condition sensing operation and/or implanting a therapeutic medical device for an extended period of time without having to replace the entire device.

For example, in a case where the reservoir 914 is capable of containing nine days of the fluid medication, but, according to regulatory measures, a single transcutaneous member cannot be maintained in the skin of the person for more than three days, a fluid delivery device such as the embodiment 900 may be utilized as follows. In the predeployment state, all the transcutaneous member devices are retracted within the housing and are not actively connected to their respective fluid paths 910. After the housing has been attached to the skin of the person, one of the three transcutaneous member devices is activated. The activation may be effected by any of the activation devices described in this application. When a transcutaneous member device is activated and the cannula 906 is driven into the skin of the person, a valve (not shown) within the injection actuator is opened, thus enabling fluid to flow from the reservoir 914 through the transcutaneous member to the person. At the end of the three day period, the person can retract the transcutaneous member, which shuts the valve, and activate a second transcutaneous member device, thereby enabling fluid to flow from the reservoir to the person through the second transcutaneous member device. This process is repeated until all of the transcutaneous member devices have been activated and then retracted. Although not specifically shown, each transcutaneous member device includes a mechanism that prevents the activation of an injection actuator that has already been activated. It will be understood that, although three transcutaneous member devices are shown in FIGS. 39A-39C, any number of transcutaneous member devices may be included in the fluid delivery device 900.

Similarly, three sensor assemblies may be incorporated into a single housing, for the purpose of carrying out a sensing operation for three days, along with the fluid dispensing operation described above. In a similar fashion as that described above, each sensing assembly would be activated individually for a certain period of time and then would be retracted and the next sensing assembly would be activated. Likewise, a plurality of therapeutic medical devices may be incorporated into a single housing, for the purpose of implanting more than one therapeutic medical device into the patient. Each therapeutic medical device would be activated individually for a certain period of time and then would be retracted and the next therapeutic medical device would be activated.

Accordingly, the present invention provides a physiologic sensing and drug and therapeutic delivery device that enables a person to conveniently and comfortably monitor a physiological condition and self-administer a treatment regimen by allowing the person to maintain a constant flow of a drug or the implantation of a therapeutic device for a period of time without having to carry multiple pieces of equipment. The device of the present invention is inexpensive to manufacture and is either disposable or semi-disposable.

The invention may be embodied in other specific forms without departing from the spirit or essential characteristics thereof. The present embodiments are therefore to be considered in respects as illustrative and not restrictive, the scope of the invention being indicated by the appended claims rather than by the foregoing description, and all changes which come within the meaning and range of the equivalency of the claims are therefore intended to be embraced therein.

1. A device for monitoring a physiological parameter of a person comprising:
a sensor device for measuring a physiological parameter associated with the person;
a processor for processing measurements of said physiological parameter generated by said sensor device;
a transcutaneous member coupled to said sensor device and said processor, including a penetrating member at a distal end thereof for piercing the skin of the person;
a housing containing said sensor device, said transcutaneous member and said processor, said housing including an exit port for receiving said distal end of said transcutaneous member upon injection of said distal end into said person and means for securing a first wall of said housing to the skin of the person; and
an injection activation device including a driving mechanism contacting said transcutaneous member for driving said penetrating member from a first position within said housing, through said exit port to a second position, external to said housing and into the skin of said person.

2. The device of claim 1 wherein at least a sample receiving portion of said sensor device is disposed at said distal end of said transcutaneous member.

3. The device of claim 2 wherein the physiological parameter is at least one of blood glucose level, blood gas level, body temperature, exposure to an external agent, allergic reactions, respiration, arrhythrnia, blood cell count, blood flow rate, average blood clotting time, thrombogenicity, blood oxygen content, blood pH and toxicity levels.

4. The device of claim 2 wherein said driving mechanism of said injection activation device comprises a plunger having a body portion extending through an aperture in a second wall of said housing and in frictional contact with said distal end of said fluid transport device, such that the application of a longitudinal force to said plunger drives said penetrating member from said first position to said second position.

5. The device of claim 4, said plunger including a friction member disposed on said body portion, said friction member causing said body portion of said plunger to have a width dimension which is slightly larger than a width dimension of said aperture of said housing, thus requiring a specific longitudinal force to be applied to said plunger to enable said friction member to pass through said aperture, said specific force being translated to said distal end of said fluid transport device.

6. The device of claim 5 wherein said friction member is an annular flange.

7. The device of claim 5, said plunger further comprising a head portion for stopping travel of said plunger by contacting said housing.

8. The device of claim 7 wherein said plunger is removable from said housing after said penetrating member is driven to said second position.

9. The device of claim 2 wherein said driving mechanism of said injection activation device comprises a plunger contained within said housing, said plunger having a first end including a lateral protrusion and a second end in frictional contact with said distal end of said transcutaneous member, said injection activation device further including a biasing spring for biasing said plunger for driving said penetrating member from said first position to said second position, and said lateral protrusion being in contact with an internal ridge of said housing, with said penetrating member in said first position, thereby preventing said plunger from driving said penetrating member from said first position to said second position, said housing including an actuator for urging said lateral protrusion from said internal ridge, thereby causing said plunger to drive said penetrating member from said first position to said second position.

10. The device of claim 9 wherein said actuator comprises a finger coupled to an inside surface of a flexible wall portion of said housing, a distal end of said finger being in contact with said lateral protrusion such that an application of pressure to said flexible wall portion causes said finger to urge said lateral protrusion from said ridge, thereby causing said plunger to drive said penetrating member from said first position to said second position.

11. The device of claim 10 wherein said distal end of said finger, upon the application of pressure to said flexible wall portion, moves in same the direction as the flexible wall portion.

12. The device of claim 10 wherein said distal end of said finger, upon the application of pressure to said flexible wall portion, moves in a substantially opposite direction as the flexible wall portion.

13. The device of claim 12 wherein said finger includes a pivot which causes the distal end of the finger to move in a direction substantially opposite that of the flexible wall portion.

14. The device of claim 2 wherein said driving mechanism of said injection activation device comprises a pivoting arm and said injection activation device further includes a latch assembly, said pivoting arm having a proximal end pivotally coupled to an inside surface of a wall of said housing and a distal end in contact with said latch assembly integral with a side wall of said housing, said fluid transport device being coupled to said arm such that when said distal end of said arm is in contact with said latch assembly, said penetrating member is in said first position;
said injection activation device further includes a biasing spring attached between said proximal and distal ends of said arm and a wall of said housing, said biasing spring urging said arm to drive said penetrating member to said second position; and
said latch assembly includes a latch for contacting said distal end of said pivoting arm to prevent said pivoting arm from driving said penetrating member from said first position to said second position under the influence of said biasing spring and a latch release mechanism for moving said latch out of contact with said distal end of said pivoting arm, thereby enabling said pivoting arm to drive said penetrating member from said first position to said second position under the influence of said biasing spring.

15. The device of claim 14 wherein said latch release mechanism includes an electrically driven actuator coupled between said latch and said side wall of said housing, such that, upon the application of a charge to said electrically driven actuator, said electrically driven actuator activates to pull said latch out of contact with said distal end of said pivoting arm.

16. The device of claim 15 wherein said electrically driven actuator comprises one of a shape memory alloy, a shape memory polymer, a piezo electric actuator and a solenoid.

17. The device of claim 15 further comprising a local processor connected to the latch release mechanism and programmed to apply a charge to said electrically driven actuator based on injection instructions; and
a wireless receiver connected to the local processor for receiving injection instructions from a separate, remote control device and delivering the injection instructions to the local processor.

18. The device of claim 17 wherein said housing is free of user input components for providing injection instructions to the local processor.

19. The device of claim 17 further comprising a remote control device separate from the transcutaneous member and including:
a remote processor;
user interface components connected to the remote processor for transmitting the injection instructions to the remote processor; and
a transmitter connected to the remote processor for transmitting the injection instructions to the receiver of the device for monitoring a physiological parameter.

20. The device of claim 14 wherein said latch release mechanism includes a mechanical lever coupled to said latch and protruding through said side wall, such that, upon said lever being pulled away from said housing, said latch is pulled out of contact with said distal end of said pivoting arm.

21. The device of claim 2 wherein said injection activation device includes a discrete secondary housing, said plunger including a first end having a lateral protrusion and a second end in frictional contact with said distal end of said fluid transport device, said second end of said plunger extending from within said secondary housing, out of a distal end thereof into said aperture of said housing and into frictional contact with said distal end of said fluid transport device;
said injection activation device further comprising a biasing spring coupled between said first end of said plunger and a proximal end of said secondary housing within said secondary housing for biasing said plunger for driving said penetrating member from said first position to said second position, said lateral protrusion being in contact with an internal ridge of said secondary housing, with said penetrating member in said first position, thereby preventing said plunger from driving said penetrating member from said first position to said second position;
said secondary housing including an actuator for urging said lateral protrusion from said internal ridge, thereby causing said plunger to drive said penetrating member from said first position to said second position.

22. The device of claim 2 wherein said injection activation device includes a discrete secondary housing, said plunger including a first end having a lateral protrusion and a second end in frictional contact with said distal end of said fluid transport device, said second end of said plunger extending from within said secondary housing, out of a distal end thereof into said aperture of said housing and into frictional contact with said distal end of said fluid transport device;
said injection activation device further comprising a biasing spring coupled between said first end of said plunger and a proximal end of said secondary housing within said secondary housing for biasing said plunger for driving said penetrating member from said first position to said second position, said lateral protrusion being in contact with a latch assembly of said secondary housing, with said penetrating member in said first position, thereby preventing said plunger from driving said penetrating member from said first position to said second position;
said latch assembly includes a latch for contacting said lateral protrusion of said plunger to prevent said plunger from driving said penetrating member from said first position to said second position under the influence of said biasing spring and a latch release mechanism coupled to said housing for moving said latch out of contact with said lateral protrusion, thereby enabling said plunger to drive said penetrating member from said first position to said second position under the influence of said biasing spring.

23. The device of claim 20 wherein said latch release mechanism includes an electrically driven actuator coupled between said latch and said side wall of said housing, such that, upon the application of a charge to said electrically driven actuator, said electrically driven actuator activates to pull said latch out of contact with said distal end of said pivoting arm.

24. The device of claim 23 wherein said electrically driven actuator comprises one of a shape memory alloy, a shape memory polymer, a piezo electric actuator and a solenoid.

25. The device of claim 23 further comprising a local processor housed in said secondary housing, said local processor being connected to the latch release mechanism and programmed to apply a charge to said electrically driven actuator based on injection instructions; and
a wireless receiver connected to the local processor for receiving injection instructions from a separate, remote control device and delivering the injection instructions to the local processor.

26. The device of claim 25 wherein said housing is free of user input components for providing injection instructions to the local processor.

27. The device of claim 25 further comprising a remote control device separate from the transcutaneous member and including:
a remote processor;
user interface components connected to the remote processor for transmitting the injection instructions to the remote processor; and
a transmitter connected to the remote processor for transmitting the injection instructions to the receiver of the device for monitoring a physiological parameter.

28. The device of claim 22 wherein said latch release mechanism includes a mechanical lever coupled to said latch and protruding through said side wall, such that, upon an application of force to said lever, said latch is moved out of contact with said distal end of said pivoting arm.

29. The device of claim 2, said driving mechanism comprising a plunger having a first end in frictional contact with said distal end of said fluid transport device, said plunger being biased to drive said penetrating member from said first position to said second position, said injection activation device further comprising a latch for contacting said plunger to maintain said penetrating member in said first position, said latch including an electrically driven actuator coupled to said latch, such that, upon the application of a charge to said electrically driven actuator, said electrically driven actuator activates to pull said latch out of contact with said plunger, thereby enabling said plunger to drive said penetrating means from said first position to said second position.

30. The device of claim 29 wherein said electrically driven actuator comprises one of a shape memory alloy, a shape memory polymer, a piezo electric actuator and a solenoid.

31. The device of claim 29 further comprising a local processor connected to the latch release mechanism and programmed to apply a charge to said electrically driven actuator based on injection instructions; and
a wireless receiver connected to the local processor for receiving injection instructions from a separate, remote control device and delivering the injection instructions to the local processor.

32. The device of claim 2 wherein said sensor device includes physiological parameter sensing means for performing a sampling operation on a sample received by said sample receiving portion to monitor a physiological parameter of the person.

33. The device of claim 32 wherein the physiological parameter is at least one of blood glucose level, blood gas level, exposure to an external agent, allergic reactions, respiration, arrhythmia, blood cell count, blood flow rate, average blood clotting time, thrombogenicity, blood oxygen content, blood pH and toxicity levels.

34. The device of claim 32 further including:
a reservoir for containing a medicine to be delivered to the person;
a fluid transport device, enclosed within said housing, for dispensing medicine from said reservoir to the person, said fluid transport device including a proximal end in fluid communication with said reservoir and a distal end having a penetrating member for piercing the skin of the person to facilitate the delivery of medicine to the person through the fluid transport device.

35. The device of claim 34 further including a second injection activation device including a second driving mechanism contacting said fluid transport device for driving said penetrating member from said first position within said housing, through said exit port to said second position, external to said housing and into the skin of said person.

36. The device of claim 35 wherein said sensor device includes means for instructing said second injection activation device to drive said fluid transport from said first position within said housing, through said exit port to said second position, external to said housing and into the skin of said person and to transport an amount of medicine to the person, based on said sampling operation.

37. A device for monitoring fluid from a person comprising:
a sensor device for receiving fluid from the person;
a fluid transport device for withdrawing fluid from the person to said sensor device, said fluid transport device including a proximal end in fluid communication with said sensor device and a distal end having a penetrating member for piercing the skin of the person to facilitate the withdrawal of fluid to the person through the fluid transport device;
a housing containing said sensor device and said fluid transport device, said housing including an exit port for receiving said distal end of said fluid transport device upon injection of said distal end into said person and means for securing a first wall of said housing to the skin of the person; and
an injection activation device including a driving mechanism contacting said fluid transport device for driving said penetrating member from a first position within said housing, through said exit port to a second position, external to said housing and into the skin of said person.

38. The device of claim 37 wherein said driving mechanism of said injection activation device comprises a plunger having a body portion extending through an aperture in a second wall of said housing and in frictional contact with said distal end of said fluid transport device, such that the application of a longitudinal force to said plunger drives said penetrating member from said first position to said second position.

39. The device of claim 38, said plunger including a friction member disposed on said body portion, said friction member causing said body portion of said plunger to have a width dimension which is slightly larger than a width dimension of said aperture of said housing, thus requiring a specific longitudinal force to be applied to said plunger to enable said friction member to pass through said aperture, said specific force being translated to said distal end of said fluid transport device.

40. The device of claim 39 wherein said friction member is an annular flange.

41. The device of claim 39, said plunger further comprising a head portion for stopping travel of said plunger by contacting said housing.

42. The device of claim 41 wherein said plunger is removable from said housing after said penetrating member is driven to said second position.

43. The device of claim 37 wherein said driving mechanism of said injection activation device comprises a plunger contained within said housing, said plunger having a first end including a lateral protrusion and a second end in frictional contact with said distal end of said fluid transport device, said injection activation device further including a biasing spring for biasing said plunger for driving said penetrating member from said first position to said second position, and said lateral protrusion being in contact with an internal ridge of said housing, with said penetrating member in said first position, thereby preventing said plunger from driving said penetrating member from said first position to said second position;
said housing including an actuator for urging said lateral protrusion from said internal ridge, thereby causing said plunger to drive said penetrating member from said first position to said second position.

44. The device of claim 43 wherein said actuator comprises a finger coupled to an inside surface of a flexible wall portion of said housing, a distal end of said finger being in contact with said lateral protrusion such that an application of pressure to said flexible wall portion causes said finger to urge said lateral protrusion from said ridge, thereby causing said plunger to drive said penetrating member from said first position to said second position.

45. The device of claim 44 wherein said distal end of said finger, upon the application of pressure to said flexible wall portion, moves in same the direction as the flexible wall portion.

46. The device of claim 44 wherein said distal end of said finger, upon the application of pressure to said flexible wall portion, moves in a substantially opposite direction as the flexible wall portion.

47. The device of claim 46 wherein said finger includes a pivot which causes the distal end of the finger to move in a direction substantially opposite that of the flexible wall portion.

48. The device of claim 37 wherein said driving mechanism of said injection activation device comprises a pivoting arm and said injection activation device further includes a latch assembly, said pivoting arm having a proximal end pivotally coupled to an inside surface of a wall of said housing and a distal end in contact with said latch assembly integral with a side wall of said housing, said fluid transport device being coupled to said arm such that when said distal end of said arm is in contact with said latch assembly, said penetrating member is in said first position;
said injection activation device further includes a biasing spring attached between said proximal and distal ends of said arm and a wall of said housing, said biasing spring urging said arm to drive said penetrating member to said second position; and
said latch assembly includes a latch for contacting said distal end of said pivoting arm to prevent said pivoting arm from driving said penetrating member from said first position to said second position under the influence of said biasing spring and a latch release mechanism for moving said latch out of contact with said distal end of said pivoting arm, thereby enabling said pivoting arm to drive said penetrating member from said first position to said second position under the influence of said biasing spring.

49. The device of claim 48 wherein said latch release mechanism includes an electrically driven actuator coupled between said latch and said side wall of said housing, such that, upon the application of a charge to said electrically driven actuator, said electrically driven actuator activates to pull said latch out of contact with said distal end of said pivoting arm.

50. The device of claim 49 wherein said electrically driven actuator comprises one of a shape memory alloy, a shape memory polymer, a piezo electric actuator and a solenoid.

51. The device of claim 49 further comprising a local processor connected to the latch release mechanism and programmed to apply a charge to said electrically driven actuator based on injection instructions; and
a wireless receiver connected to the local processor for receiving injection instructions from a separate, remote control device and delivering the injection instructions to the local processor.

52. The device of claim 51 wherein said housing is free of user input components for providing injection instructions to the local processor.

53. The device of claim 51 further comprising a remote control device separate from the fluid delivery device and including:
a remote processor;
user interface components connected to the remote processor for transmitting the injection instructions to the remote processor; and
a transmitter connected to the remote processor for transmitting the injection instructions to the receiver of the fluid delivery device.

54. The device of claim 48 wherein said latch release mechanism includes a mechanical lever coupled to said latch and protruding through said side wall, such that, upon said lever being pulled away from said housing, said latch is pulled out of contact with said distal end of said pivoting arm.

55. The device of claim 38 wherein said injection activation device includes a discrete secondary housing, said plunger including a first end having a lateral protrusion and a second end in frictional contact with said distal end of said fluid transport device, said second end of said plunger extending from within said secondary housing, out of a distal end thereof into said aperture of said housing and into frictional contact with said distal end of said fluid transport device;
said injection activation device further comprising a biasing spring coupled between said first end of said plunger and a proximal end of said secondary housing within said secondary housing for biasing said plunger for driving said penetrating member from said first position to said second position, said lateral protrusion being in contact with an internal ridge of said secondary housing, with said penetrating member in said first position, thereby preventing said plunger from driving said penetrating member from said first position to said second position;
said secondary housing including an actuator for urging said lateral protrusion from said internal ridge, thereby causing said plunger to drive said penetrating member from said first position to said second position.

56. The device of claim 38 wherein said injection activation device includes a discrete secondary housing, said plunger including a first end having a lateral protrusion and a second end in frictional contact with said distal end of said fluid transport device, said second end of said plunger extending from within said secondary housing, out of a distal end thereof into said aperture of said housing and into frictional contact with said distal end of said fluid transport device;
said injection activation device further comprising a biasing spring coupled between said first end of said plunger and a proximal end of said secondary housing within said secondary housing for biasing said plunger for driving said penetrating member from said first position to said second position, said lateral protrusion being in contact with a latch assembly of said secondary housing, with said penetrating member in said first position, thereby preventing said plunger from driving said penetrating member from said first position to said second position;
said latch assembly includes a latch for contacting said lateral protrusion of said plunger to prevent said plunger from driving said penetrating member from said first position to said second position under the influence of said biasing spring and a latch release mechanism coupled to said housing for moving said latch out of contact with said lateral protrusion, thereby enabling said plunger to drive said penetrating member from said first position to said second position under the influence of said biasing spring.

57. The device of claim 54 wherein said latch release mechanism includes an electrically driven actuator coupled between said latch and said side wall of said housing, such that, upon the application of a charge to said electrically driven actuator, said electrically driven actuator activates to pull said latch out of contact with said distal end of said pivoting arm.

58. The device of claim 57 wherein said electrically driven actuator comprises one of a shape memory alloy, a shape memory polymer, a piezo electric actuator and a solenoid.

59. The device of claim 57 further comprising a local processor housed in said secondary housing, said local processor being connected to the latch release mechanism and programmed to apply a charge to said electrically driven actuator based on injection instructions; and
a wireless receiver connected to the local processor for receiving injection instructions from a separate, remote control device and delivering the injection instructions to the local processor.

60. The device of claim 59 wherein said housing is free of user input components for providing injection instructions to the local processor.

61. The device of claim 59 further comprising a remote control device separate from the fluid delivery device and including:
a remote processor;
user interface components connected to the remote processor for transmitting the injection instructions to the remote processor; and
a transmitter connected to the remote processor for transmitting the injection instructions to the receiver of the fluid delivery device.

62. The device of claim 56 wherein said latch release mechanism includes a mechanical lever coupled to said latch and protruding through said side wall, such that, upon an application of force to said lever, said latch is moved out of contact with said distal end of said pivoting arm.

63. The device of claim 37, said driving mechanism comprising a plunger having a first end in frictional contact with said distal end of said fluid transport device, said plunger being biased to drive said penetrating member from said first position to said second position, said injection activation device further comprising a latch for contacting said plunger to maintain said penetrating member in said first position, said latch including an electrically driven actuator coupled to said latch, such that, upon the application of a charge to said electrically driven actuator, said electrically driven actuator activates to pull said latch out of contact with said plunger, thereby enabling said plunger to drive said penetrating means from said first position to said second position.

64. The device of claim 63 wherein said electrically driven actuator comprises one of a shape memory alloy, a shape memory polymer, a piezo electric actuator and a solenoid.

65. The device of claim 63 further comprising a local processor connected to the latch release mechanism and programmed to apply a charge to said electrically driven actuator based on injection instructions; and
a wireless receiver connected to the local processor for receiving injection instructions from a separate, remote control device and delivering the injection instructions to the local processor.

66. The device of claim 37 wherein said sensor device includes physiological parameter sensing means for performing a sampling operation on the fluid to monitor a physiological parameter of the person.

67. The device of claim 66 wherein the physiological parameter is at least one of blood glucose level, blood gas level exposure to an external agent, allergic reactions, respiration, arrhythmia, blood cell count, blood flow rate, average blood clotting time, thrombogenicity, blood oxygen content, blood pH and toxicity levels.

68. The device of claim 66 further including:
a reservoir for containing a medicine to be delivered to the person;
a second fluid transport device, enclosed within said housing, for dispensing medicine from said reservoir to the person, said fluid transport device including a proximal end in fluid communication with said reservoir and a distal end having a penetrating member for piercing the skin of the person to facilitate the delivery of medicine to the person through the fluid transport device.

69. The device of claim 68 further including a second injection activation device including a second driving mechanism contacting said second fluid transport device for driving said penetrating member from said first position within said housing, through said exit port to said second position, external to said housing and into the skin of said person.

70. The device of claim 69 wherein said sensor device includes means for instructing said second injection activation device to drive said second fluid transport from said first position within said housing, through said exit port to said second position, external to said housing and into the skin of said person and to transport an amount of medicine to the person, based on said sampling operation.

71. A device for monitoring a physiological parameter of a person comprising:
a sensor device for measuring a physiological parameter associated with the person;
a processor for processing measurements of said physiological parameter generated by said sensor device;
a transcutaneous member coupled to said sensor device and said processor, including a proximal end, a penetrating member at a distal end thereof for piercing the skin of the person and a medial portion disposed between said proximal and distal ends;
a housing containing said sensor device, said transcutaneous member and said processor, said housing including an exit port for receiving said distal end of said transcutaneous member upon injection of said penetrating member into said person and means for securing a first wall of said housing to the skin of the person; and
an injection activation device including a driving mechanism contacting said proximal end of said transcutaneous member for driving said penetrating member from a first position within said housing, through said exit port to a second position, external to said housing and into the skin of said person;
wherein said medial portion is disposed substantially parallel to said first wall of said housing, said transcutaneous member including a retention device which, with said penetrating member in said first position, is biased against a latch assembly of said injection activation device by a biasing spring of said injection activation device, which is coupled between said retention device and an internal ridge of said housing, said biasing spring being in an energized state such that, upon activating said latch assembly, said biasing spring drives said transcutaneous member in a direction of travel substantially parallel to said first wall, resulting in said penetrating member being driven from said first position to said second position.

72. The device of claim 71 wherein said distal end of said transcutaneous member is flexible; and
said housing includes a deflecting device in the path of travel of said transcutaneous member;
wherein, upon activating said latch assembly, said distal end of said transcutaneous member contacts said deflecting device which causes said distal end of said transcutaneous member to be deflected from said direction of travel substantially parallel to said first wall of said housing to a second direction of travel at an angle of at least 15.

73. The device of claim 72 wherein said second direction of travel is up to 90.

74. The device of claim 72 wherein said latch assembly includes a latch for contacting said retention device of said transcutaneous member to prevent said biasing spring from driving said penetrating member from said first position to said second position and a latch release mechanism coupled to said housing for moving said latch out of contact with said retention device, thereby enabling said biasing spring to drive said penetrating member from said first position to said second position.

75. The device of claim 74 wherein said latch release mechanism includes an electrically driven actuator coupled between said latch and said housing, such that, upon the application of a charge to said electrically driven actuator, said shape memory allow wire contracts, pulling said latch out of contact with said retention device of said transcutaneous member.

76. The device of claim 75 wherein said electrically driven actuator comprises one of a shape memory alloy, a shape memory polymer, a piezo electric actuator and a solenoid.

77. The device of claim 75 wherein said housing further includes a local processor connected to the latch release mechanism and programmed to apply a charge to said electrically driven actuator based on injection instructions; and
a wireless receiver connected to the local processor for receiving injection instructions from a separate, remote control device and delivering the injection instructions to the local processor

78. The device of claim 77 wherein said housing is free of user input components for providing injection instructions to the local processor.

79. The device of claim 78 further comprising a remote control device separate from the housing and including:
a remote processor;
user interface components connected to the remote processor for transmitting the injection instructions to the remote processor; and
a transmitter connected to the remote processor for transmitting the injection instructions to the receiver within said housing.

80. The device of claim 74 wherein said latch release mechanism includes a mechanical lever coupled to said latch and protruding through said side wall, such that, upon an application of force to said lever, said latch is moved out of contact with said retention device.

81. The device of claim 71 wherein said biasing spring comprises one of a torsional spring, a coil spring, a helical spring, a compression spring, an extension spring, an air spring, a wave spring, a conical spring, a constant force spring, a belleville spring and a beehive spring.

82. The device of claim 71 wherein the physiological parameter is at least one of blood glucose level, blood gas level exposure to an external agent and allergies.

83. The device of claim 71 further including:
a reservoir for containing a medicine to be delivered to the person;
a fluid transport device, enclosed within said housing, for dispensing medicine from said reservoir to the person, said fluid transport device including a proximal end in fluid communication with said reservoir and a distal end having a penetrating member for piercing the skin of the person to facilitate the delivery of medicine to the person through the fluid transport device.

84. The device of claim 83 further including a second injection activation device including a second driving mechanism contacting said second fluid transport device for driving said penetrating member from a third position within said housing, through said exit port to said fourth position, external to said housing and into the skin of said person.

85. The device of claim 84 wherein said sensor device includes means for instructing said second injection activation device to drive said second fluid transport from said third position within said housing, through said exit port to said fourth position, external to said housing and into the skin of said person and to transport an amount of medicine to the person, based on said physiological parameter sensed by said sensor device.

86. An ambulatory medical device comprising:
a transcutaneous member including a penetrating member at a distal end thereof for piercing the skin of the person;
a therapeutic element coupled to said transcutaneous member for administering a treatment to a person;
a housing containing said therapeutic element and said transcutaneous member, said housing including an exit port for receiving said distal end of said transcutaneous member upon injection of said distal end into said person and means for securing a first wall of said housing to the skin of the person; and
an injection activation device including a driving mechanism contacting said transcutaneous member for driving said penetrating member from a first position within said housing, through said exit port to a second position, external to said housing and into the skin of said person.

87. The ambulatory device of claim 86 wherein said treatment is initiated upon said penetrating member being driven into the skin of the person.

88. The ambulatory device of claim 86 further comprising a processor for controlling said injection activation device.

89. The ambulatory device of claim 86 wherein said therapeutic element comprises at least one of pacemaker leads, defibrillator leads, time-release solid-form drugs, magnets, electromagnets, radioactive seeds, thermal elements and one or more transcutaneous electrode nerve stimulus (“TENS”) devices.

90. The ambulatory device of claim 89 wherein said driving mechanism of said injection activation device comprises a plunger having a body portion extending through an aperture in a second wall of said housing and in frictional contact with said distal end of said transcutaneous member, such that the application of a longitudinal force to said plunger drives said penetrating member from said first position to said second position.

91. The device of claim 90, said plunger including a friction member disposed on said body portion, said friction member causing said body portion of said plunger to have a width dimension which is slightly larger than a width dimension of said aperture of said housing, thus requiring a specific longitudinal force to be applied to said plunger to enable said friction member to pass through said aperture, said specific force being translated to said distal end of said transcutaneous member.

92. The device of claim 91 wherein said friction member is an annular flange.

93. The device of claim 91, said plunger further comprising a head portion for stopping travel of said plunger by contacting said housing.

94. The device of claim 93 wherein said plunger is removable from said housing after said penetrating member is driven to said second position.

95. The device of claim 86 wherein said driving mechanism of said injection activation device comprises a plunger contained within said housing, said plunger having a first end including a lateral protrusion and a second end in frictional contact with said distal end of said transcutaneous member, said injection activation device further including a biasing spring for biasing said plunger for driving said penetrating member from said first position to said second position, and said lateral protrusion being in contact with an internal ridge of said housing, with said penetrating member in said first position, thereby preventing said plunger from driving said penetrating member from said first position to said second position;
said housing including an actuator for urging said lateral protrusion from said internal ridge, thereby causing said plunger to drive said penetrating member from said first position to said second position.

96. The device of claim 95 wherein said actuator comprises a finger coupled to an inside surface of a flexible wall portion of said housing, a distal end of said finger being in contact with said lateral protrusion such that an application of pressure to said flexible wall portion causes said finger to urge said lateral protrusion from said ridge, thereby causing said plunger to drive said penetrating member from said first position to said second position.

97. The device of claim 96 wherein said distal end of said finger, upon the application of pressure to said flexible wall portion, moves in same the direction as the flexible wall portion.

98. The device of claim 96 wherein said distal end of said finger, upon the application of pressure to said flexible wall portion, moves in a substantially opposite direction as the flexible wall portion.

99. The device of claim 98 wherein said finger includes a pivot which causes the distal end of the finger to move in a direction substantially opposite that of the flexible wall portion.

100. The device of claim 86 wherein said driving mechanism of said injection activation device comprises a pivoting arm and said injection activation device further includes a latch assembly, said pivoting arm having a proximal end pivotally coupled to an inside surface of a wall of said housing and a distal end in contact with said latch assembly integral with a side wall of said housing, said transcutaneous member being coupled to said arm such that when said distal end of said arm is in contact with said latch assembly, said penetrating member is in said first position;
said injection activation device further includes a biasing spring attached between said proximal and distal ends of said arm and a wall of said housing, said biasing spring urging said arm to drive said penetrating member to said second position; and
said latch assembly includes a latch for contacting said distal end of said pivoting arm to prevent said pivoting arm from driving said penetrating member from said first position to said second position under the influence of said biasing spring and a latch release mechanism for moving said latch out of contact with said distal end of said pivoting arm, thereby enabling said pivoting arm to drive said penetrating member from said first position to said second position under the influence of said biasing spring.

101. The device of claim 100 wherein said latch release mechanism includes an electrically driven actuator coupled between said latch and said side wall of said housing, such that, upon the application of a charge to said electrically driven actuator, said electrically driven actuator activates to pull said latch out of contact with said distal end of said pivoting arm.

102. The device of claim 101 wherein said electrically driven actuator comprises one of a shape memory alloy, a shape memory polymer, a piezo electric actuator and a solenoid.

103. The device of claim 101 further comprising a local processor connected to the latch release mechanism and programmed to apply a charge to said electrically driven actuator based on injection instructions; and
a wireless receiver connected to the local processor for receiving injection instructions from a separate, remote control device and delivering the injection instructions to the local processor.

104. The device of claim 103 wherein said housing is free of user input components for providing injection instructions to the local processor.

105. The device of claim 103 further comprising a remote control device separate from the fluid delivery device and including:
a remote processor;
user interface components connected to the remote processor for transmitting the injection instructions to the remote processor; and
a transmitter connected to the remote processor for transmitting the injection instructions to the receiver of said transcutaneous member.

106. The device of claim 100 wherein said latch release mechanism includes a mechanical lever coupled to said latch and protruding through said side wall, such that, upon said lever being pulled away from said housing, said latch is pulled out of contact with said distal end of said pivoting arm.

107. The device of claim 86 wherein said injection activation device includes a discrete secondary housing, said plunger including a first end having a lateral protrusion and a second end in frictional contact with said distal end of said transcutaneous member, said second end of said plunger extending from within said secondary housing, out of a distal end thereof into said aperture of said housing and into frictional contact with said distal end of said transcutaneous member;
said injection activation device further comprising a biasing spring coupled between said first end of said plunger and a proximal end of said secondary housing within said secondary housing for biasing said plunger for driving said penetrating member from said first position to said second position, said lateral protrusion being in contact with an internal ridge of said secondary housing, with said penetrating member in said first position, thereby preventing said plunger from driving said penetrating member from said first position to said second position;
said secondary housing including an actuator for urging said lateral protrusion from said internal ridge, thereby causing said plunger to drive said penetrating member from said first position to said second position.

108. The device of claim 86 wherein said injection activation device includes a discrete secondary housing, said plunger including a first end having a lateral protrusion and a second end in frictional contact with said distal end of said transcutaneous member, said second end of said plunger extending from within said secondary housing, out of a distal end thereof into said aperture of said housing and into frictional contact with said distal end of said transcutaneous member;
said injection activation device further comprising a biasing spring coupled between said first end of said plunger and a proximal end of said secondary housing within said secondary housing for biasing said plunger for driving said penetrating member from said first position to said second position, said lateral protrusion being in contact with a latch assembly of said secondary housing, with said penetrating member in said first position, thereby preventing said plunger from driving said penetrating member from said first position to said second position;
said latch assembly includes a latch for contacting said lateral protrusion of said plunger to prevent said plunger from driving said penetrating member from said first position to said second position under the influence of said biasing spring and a latch release mechanism coupled to said housing for moving said latch out of contact with said lateral protrusion, thereby enabling said plunger to drive said penetrating member from said first position to said second position under the influence of said biasing spring.

109. The device of claim 106 wherein said latch release mechanism includes an electrically driven actuator coupled between said latch and said side wall of said housing, such that, upon the application of a charge to said electrically driven actuator, said electrically driven actuator activates to pull said latch out of contact with said distal end of said pivoting arm.

110. The device of claim 109 wherein said electrically driven actuator comprises one of a shape memory alloy, a shape memory polymer, a piezo electric actuator and a solenoid.

111. The device of claim 109 further comprising a local processor housed in said secondary housing, said local processor being connected to the latch release mechanism and programmed to apply a charge to said electrically driven actuator based on injection instructions; and
a wireless receiver connected to the local processor for receiving injection instructions from a separate, remote control device and delivering the injection instructions to the local processor.

112. The device of claim 111 wherein said housing is free of user input components for providing injection instructions to the local processor.

113. The device of claim 111 further comprising a remote control device separate from the fluid delivery device and including:
a remote processor;
user interface components connected to the remote processor for transmitting the injection instructions to the remote processor; and
a transmitter connected to the remote processor for transmitting the injection instructions to the receiver of said transcutaneous member.

114. The device of claim 108 wherein said latch release mechanism includes a mechanical lever coupled to said latch and protruding through said side wall, such that, upon an application of force to said lever, said latch is moved out of contact with said distal end of said pivoting arm.

115. The device of claim 86, said driving mechanism comprising a plunger having a first end in frictional contact with said distal end of said transcutaneous member, said plunger being biased to drive said penetrating member from said first position to said second position, said injection activation device further comprising a latch for contacting said plunger to maintain said penetrating member in said first position, said latch including an electrically driven actuator coupled to said latch, such that, upon the application of a charge to said electrically driven actuator, said electrically driven actuator activates to pull said latch out of contact with said plunger, thereby enabling said plunger to drive said penetrating means from said first position to said second position.

116. The device of claim 115 wherein said electrically driven actuator comprises one of a shape memory alloy, a shape memory polymer, a piezo electric actuator and a solenoid.

117. The device of claim 115 further comprising a local processor connected to the latch release mechanism and programmed to apply a charge to said electrically driven actuator based on injection instructions; and
a wireless receiver connected to the local processor for receiving injection instructions from a separate, remote control device and delivering the injection instructions to the local processor.

118. The device of claim I wherein said processor includes an injection activation instruction generation portion for providing injection activation instructions to said injection activation device based on a trigger signal provided to said injection activation instruction generation portion.

119. The device of claim 118 wherein said trigger signal is generated within said processor based on timing instructions programmed into said processor, said timing instructions causing said trigger signal to be provided to said injection activation instruction generation portion at predetermined time intervals.

120. The device of claim 119 wherein said trigger signal is generated within said processor based on a sensor input to said processor from a second sensor which monitors at least one environmental parameter, said sensor input causing said trigger signal to be provided to said injection activation instruction generation portion upon said environmental parameter reaching a predetermined level.

121. The device of claim 120 wherein said second sensor is disposed within said housing.

122. The device of claim 121 wherein said second sensor is located externally from said housing.

123. The device of claim 122 wherein said second sensor includes a transmitter for transmitting said sensor input to a receiver associated with said processor.

124. The device of claim 120 wherein said environmental parameter includes at least one of temperature, pressure, oxygen level, light and the presence of a chemical agent.

125. A device for monitoring a parameter of a person comprising:
a sensor device for measuring a parameter associated with the person;
a processor for processing measurements of said parameter generated by said sensor device;
a first transcutaneous member coupled to said sensor device and said processor, including a first penetrating member at a distal end thereof for piercing the skin of the person;
a reservoir for containing a medicine to be delivered to the person;
a fluid transport device for dispensing medicine from said reservoir to the person, said fluid transport device including a second transcutaneous member including a proximal end in fluid communication with said reservoir and a distal end having a second penetrating member for piercing the skin of the person to facilitate the delivery of medicine to the person through the fluid transport device;
a housing containing said sensor device, said first transcutaneous member, said reservoir, said fluid transport device and said processor, said housing including an exit ports for receiving said distal ends of said first and second transcutaneous members upon injection of said distal ends into said person and means for securing a first wall of said housing to the skin of the person;
a first injection activation device including a driving mechanism contacting said first transcutaneous member for driving said first penetrating member from a first position within said housing, through said exit port to a second position, external to said housing and into the skin of said person; and
a second injection activation device including a second driving mechanism contacting said second transcutaneous member for driving said second penetrating member from said first position within said housing, through said exit port to said second position, external to said housing and into the skin of said person.

126. The device of claim 125 wherein said processor includes an injection activation instruction generation portion for providing injection activation instructions to said first and second injection activation devices based on a trigger signal provided to said injection activation instruction generation portion.

127. The device of claim 126 wherein said trigger signal is generated within said processor based on timing instructions programmed into said processor, said timing instructions causing said trigger signal to be provided to said injection activation instruction generation portion at predetermined time intervals.

128. The device of claim 126 wherein said trigger signal is generated within said processor based on a sensor input to said processor from a second sensor which monitors at least one environmental parameter, said sensor input causing said trigger signal to be provided to said injection activation instruction generation portion upon said environmental parameter reaching a predetermined level.

129. The device of claim 126 wherein said second sensor is disposed within said housing.

130. The device of claim 126 wherein said second sensor is located externally from said housing.

131. The device of claim 130 wherein said second sensor includes a transmitter for transmitting said sensor input to a receiver associated with said processor.

132. The device of claim 126 wherein said environmental parameter includes at least one of temperature, pressure, oxygen level, light and the presence of a chemical agent.

133. The device of claim 128 wherein said processor provides injection activation instructions to said first injection activation device when said second sensor determines that said at least one environmental parameter has reached said predetermined level.

134. The device of claim 128 wherein said processor provides injection activation instructions to said second injection activation device when said second sensor determines that said at least one environmental parameter has reached said predetermined level.

135. The device of claim 128 wherein said sensor device monitors a physiological parameter associated with the person; and
said processor provides first injection activation instructions to said first injection activation device when said second sensor determines that said at least one environmental parameter has reaches said predetermined level and provides second injection activation instructions to said second injection activation device when said sensor device determines that said physiological parameter has reached a predetermined level.
